SEQUENCE LISTING (53 sequences)

SEQ ID NO: 1 - protein, 31 aa, Homo sapiens
HAEGTFTSDVSSYLEGQAAKEFIAWLVKGRG

SEQ ID NO: 2 - protein, 21 aa, Homo sapiens
GIVEQCCTSICSLYQLENYCN

SEQ ID NO: 3 - protein, 12 aa, Artificial Sequence
  source: /note=“Description of Artificial Sequence: Synthetic peptide”
GGGSGGGSGGGS

SEQ ID NO: 4 - protein, 8 aa, Artificial Sequence
  source: /note=“Description of Artificial Sequence: Synthetic peptide”
YPYDVPDY

SEQ ID NO: 5 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 6 - protein, 160 aa, Homo sapiens
RSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 7 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 8 - protein, 32 aa, Homo sapiens
SPKMVQGSGCFGRKMDRISSSSGLGCKVLRRH

SEQ ID NO: 9 - protein, 29 aa, Homo sapiens
DSWMEEVIKLCGRELVRAQIAICGMSTWS

SEQ ID NO: 10 - protein, 527 aa, Homo sapiens
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 11 - protein, 527 aa, Artificial Sequence; identical to SEQ ID NO: 10
  source: /note=“Description of Artificial Sequence: Synthetic polypeptide”

SEQ ID NO: 12 - protein, 355 aa, Artificial Sequence
  source: /note=“Description of Artificial Sequence: Synthetic polypeptide”
SYQGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 13 - protein, 527 aa, Artificial Sequence
  source: /note=“Description of Artificial Sequence: Synthetic polypeptide”
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGNWSTAESGAECTQWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAAAAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 14 - protein, 276 aa, Homo sapiens
DSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 15 - protein, 452 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 215 aa, Artificial Sequence
  source: /note=“Description of Artificial Sequence: Synthetic polypeptide”
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 34 aa, Homo sapiens
SVSEIQLMHNLGKHLNSMERVEWLRKKLQDVHNF

SEQ ID NO: 18 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 19 - protein, 515 aa, Homo sapiens
MVLSQEEPDSARGTSEAQPLGPAPTGAAPPPGPGPSDSPEAAVEKVEVELAGPATAEPHEPPEPPEGGWGWLVMLAAMWCNGSVFGIQNACGVLFVSMLETFGSKDDDKMVFKTAWVGSLSMGMIFFCCPIVSVFTDLFGCRKTAVVGAAVGFVGLMSSSFVSSIEPLYLTYGIIFACGCSFAYQPSLVILGHYFKKRLGLVNGIVTAGSSVFTILLPLLLRVLIDSVGLFYTLRVLCIFMFVLFLAGFTYRPLATSTKDKESGGSGSSLFSRKKFSPPKKIFNFAIFKVTAYAVWAVGIPLALFGYFVPYVHLMKHVNERFQDEKNKEVVLMCIGVTSGVGRLLFGRIADYVPGVKKVYLQVLSFFFIGLMSMMIPLCSIFGALIAVCLIMGLFDGCFISIMAPIAFELVGAQDVSQAIGFLLGFMSIPMTVGPPIAGLLRDKLGSYDVAFYLAGVPPLIGGAVLCFIPWIHSKKQREISKTTGKEKMEKMLENQNSLLSSSSGMFKKESDSII

SEQ ID NO: 20 - protein, 551 aa, Homo sapiens
MPSETPQAEVGPTGCPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLRVQELGLSAPLTVLPTITCGHTIEILREKGFDQAPVVDEAGVILGMVTLGNMLSSLLAGKVQPSDQVGKVIYKQFKQIRLTDTLGRLSHILEMDHFALVVHEQIQYHSTGKSSQRQMVFGVVTAIDLLNFVAAQERDQK

SEQ ID NO: 21 - protein, 505 aa, Homo sapiens
MKKAEMGRFSISPDEDSSSYSSNSDFNYSYPTKQAALKSHYADVDPENQNFLLESNLGKKKYETEFHPGTTSFGMSVFNLSNAIVGSGILGLSYAMANTGIALFIILLTFVSIFSLYSVHLLLKTANEGGSLLYEQLGYKAFGLVGKLAASGSITMQNIGAMSSYLFIVKYELPLVIQALTNIEDKTGLWYLNGNYLVLLVSLVVILPLSLFRNLGYLGYTSGLSLLCMVFFLIVVICKKFQVPCPVEAALIINETINTTLTQPTALVPALSHNVTENDSCRPHYFIFNSQTVYAVPILIFSFVCHPAVLPIYEELKDRSRRRMMNVSKISFFAMFLMYLLAALFGYLTFYEHVESELLHTYSSILGTDILLLIVRLAVLMAVTLTVPVVIFPIRSSVTHLLCASKDFSWWRHSLITVSILAFTNLLVIFVPTIRDIFGFIGASAASMLIFILPSAFYIKLVKKEPMKSVQKIGALFFLLSGVLVMTGSMALIVLDWVHNPGGGH

SEQ ID NO: 22 - protein, 532 aa, Homo sapiens
MEKSNETNGYLDSAQAGPAAGPGAPGTAAGRARRCAGFLRRQALVLLTVSGVLAGAGLGAALRGLSLSRTQVTYLAFPGEMLLRMLRMIILPLVVCSLVSGAASLDASCLGRLGGIAVAYFGLTTLSASALAVALAFIIKPGSGAQTLQSSDLGLEDSGPPPVPKETVDSFLDLARNLFPSNLVVAAFRTYATDYKVVTQNSSSGNVTHEKIPIGTEIEGMNILGLVLFALVLGVALKKLGSEGEDLIRFFNSLNEATMVLVSWIMWYVPVGIMFLVGSKIVEMKDIIVLVTSLGKYIFASILGHVIHGGIVLPLIYFVFTRKNPFRFLLGLLAPFATAFATCSSSATLPSMMKCIEENNGVDKRISRFILPIGATVNMDGAAIFQCVAAVFIAQLNNVELNAGQIFTILVTATASSVGAAGVPAGGVLTIAIILEAIGLPTHDLPLILAVDWIVDRTTTVVNVEGDALGAGILHHLNQKATKKGEQELAEVKVEAIPNCKSEEETSPLVTHQNPAGPVASAPELESKESVL

SEQ ID NO: 23 - protein, 302 aa, Aspergillus flavus
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKL

SEQ ID NO: 24 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 25 - protein, 448 aa, Homo sapiens [sequence omitted]

SEQ ID NO: 26 - protein, 406 aa, Homo sapiens [sequence omitted]

SEQ ID NO: 27 - protein, 248 aa, Artificial Sequence
  source: /note=“Description of Artificial Sequence: Synthetic polypeptide”
EVQLVESGGGLVKPGGSLRLSCAASGFTFSNYAMSWVRQTPEKRLEWVATISRSGSYSYFPDSVQGRFTISRDNAKNSLYLQMNSLRAEDTAVYYCARLGGYDEGDAMDSWGQGTTVTVSSGGGGSGGGGSGGGGSDIVMTQTPLSLSVTPGQPASISCKSSQSLLESDGKTYLNWYLQKPGQSPQLLIYLVSILDSGVPDRFSGSGSGTDFTLKISRVEAEDVGVYYCLQATHFPQTFGGGTKVEIK

SEQ ID NO: 28 - protein, 247 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAILGATSLSAFDIWGQGTMVTVSSGGGGSGGGGSGGGGSQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSGVFGGGTKLTVL

SEQ ID NO: 29 - protein, 241 aa, Artificial Sequence
  source: /note=“Description of Artificial Sequence: Synthetic polypeptide”
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKGGGGSGGGGSGGGGSQVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSS

SEQ ID NO: 30 - protein, 245 aa, Artificial Sequence
  source: /note=“Description of Artificial Sequence: Synthetic polypeptide”
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKGGGGSGGGGSGGGGSQVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSS

SEQ ID NO: 31 - protein, 24 aa, Homo sapiens
QLYSALANKCCHVGCTKRSLARFC

SEQ ID NO: 32 - protein, 5 aa, Unknown
  source: /note=“Description of Unknown: sortase transfer signature peptide”
  MOD_RES (3)..(3): Any amino acid
LPXTG

SEQ ID NO: 33 - protein, 131 aa, Artificial Sequence
  source: /note=“Description of Artificial Sequence: Synthetic polypeptide”
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 34 - protein, 93 aa, Homo sapiens
VSDVPRDLEWAATPTSLLISWDAPAVTVRYYRITYGETGGNSPVQEFTVPGSKSTATISGLKPGVDYTITGYAVTGRGDSPASSKPISINYRT

SEQ ID NO: 35 - protein, 312 aa, Artificial Sequence
  source: /note=“Description of Artificial Sequence: Synthetic polypeptide”
DSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 36 - protein, 8 aa, Artificial Sequence
  source: /note=“Description of Artificial Sequence: Synthetic peptide”
GGSGGSGG

SEQ ID NO: 37 - protein, 8 aa, Artificial Sequence
  source: /note=“Description of Artificial Sequence: Synthetic peptide”
GGGSGGGS

SEQ ID NO: 38 - protein, 19 aa, Artificial Sequence
  source: /note=“Description of Artificial Sequence: Synthetic peptide”
MYGKIIFVLLLSEIVSISA

SEQ ID NO: 39 - protein, 16 aa, Homo sapiens
CPPCPAPELLGGPSVF

SEQ ID NO: 40 - protein, 15 aa, Homo sapiens
CPPCPAPPVAGPSVF

SEQ ID NO: 41 - protein, 16 aa, Homo sapiens
CPRCPAPELLGGPSVF

SEQ ID NO: 42 - protein, 16 aa, Homo sapiens
AHHAQAPEFLGGPSVF

SEQ ID NO: 43 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 44 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRRGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 45 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGIKINSWESSRRGHSFLSNLHLRNGELVIHEKGFYYIYSQTYFRFQEEIKENTKNDKQMVQYIYKYTDYPDPILLMKSARNSCWSKDAEYGLYSIYQGGIFELKENDRIFVSVTNEHLIDMDHEASFFGAFLVG

SEQ ID NO: 46 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 47 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQERIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 48 - protein, 243 aa, Artificial Sequence
  source: /note=“Description of Artificial Sequence: Synthetic polypeptide”
EVQLVESGGGLVQPGGSLRLSCAASGFNFNDYFMNWVRQAPGKGLEWVAQMRNKNYQYGTYYAESLEGRFTISRDDSKNSLYLQMNSLKTEDTAVYYCARESYYGFTSYWGQGTLVTVSSGGGGSGGGGSGGGGSDIQMTQSPSSLSASVGDRVTITCQASQDIGISLSWYQQKPGKAPKLLIYNANNLADGVPSRFSGSGSGTDFTLTISSLQPEDFATYYCLQHNSAPYTFGQGTKLEIKR

SEQ ID NO: 49 - protein, 243 aa, Artificial Sequence
  source: /note=“Description of Artificial Sequence: Synthetic polypeptide”
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 50 - skipped

SEQ ID NO: 51 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 52 - protein, 239 aa, Artificial Sequence
  source: /note=“Description of Artificial Sequence: Synthetic polypeptide”
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSGGGGSGGGGSGGGGSESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 53 - protein, 30 aa, Homo sapiens
FVNQHLCGSHLVEALYLVCGERGFFYTPKT

(19) United States
(12) Patent Application Publication
(10) Pub. No.: US 2020/0172867 A1
(43) Pub. Date: Jun. 4, 2020

(54) COMPOSITIONS AND METHODS RELATED TO MULTIMODAL THERAPEUTIC CELL SYSTEMS FOR CARDIOMETABOLIC DISEASE

(71) Applicant: RUBIUS THERAPEUTICS, INC., Cambridge, MA (US)

(72) Inventors: Tom Wickham, Groton, MA (US); Tiffany F. Chen, Cambridge, MA (US); Torben Straight Nissen, Chestnut Hill, MA (US); Nathan Dowden, Winchester, MA (US); Robert J. Deans, Riverside, CA (US); John Round, Cambridge, MA (US); Avak Kahvejian, Lexington, MA (US); Jordi Mata-Fink, Baltimore, MD (US); Noubar B. Afeyan, Lexington, MA (US)

(21) Appl. No.: 16/632,115

(22) PCT Filed: Jul. 19, 2017
(86) PCT No.: PCT/US2017/042861

§ 371 Date: Jan. 17, 2020

(51) Int. Cl. C12N 5/078; C12N 15/86; A61K 35/18; A61P 3/04; A61P 9/04; A61P 5/50

(52) U.S. Cl. CPC C12N 5/0641; C12N 15/86; A61K 35/18; C12N 2740/15043; A61P 9/04; A61P 5/50; C12N 2510/00; A61P 3/04

(57) ABSTRACT

The invention includes compositions and methods related to multimodal therapies, e.g., for treating cardiovascular diseases and metabolic disorders. A multimodal therapy described herein provides and/or administers a plurality of agents that function in a coordinated manner to provide a therapeutic benefit to a subject in need thereof, e.g., a subject having a cardiovascular disease or metabolic disorder.

### BACKGROUND

Red blood cells have been considered for use as drug delivery systems, e.g., to degrade toxic metabolites or inactivate xenobiotics, and in other biomedical applications.

### SUMMARY OF THE INVENTION

The invention includes compositions and methods related to multimodal therapies. The therapies are useful, e.g., for treating cardiovascular or metabolic diseases. A multimodal therapy described herein provides and/or administers a plurality of agents that function in a coordinated manner to provide a therapeutic benefit to a subject in need thereof, e.g., a subject having a cardiovascular or metabolic disease. In general, a multimodal therapy described herein includes the administration to a subject of a preparation of engineered erythroid cells, e.g., enucleated erythroid cell, comprising (e.g., expressing or containing) a plurality of agents (e.g., polypeptides) that function in a coordinated manner (e.g., agent-additive, agent-synergistic, multiplicative, independent function, localization-based, proximity-dependent, scaffold-based, multimer-based, pathway-based, or compensatory).

The present disclosure provides, in some aspects, enucleated erythroid cell, comprising a first exogenous polypeptide comprising a first cardiovascular therapeutic, and a second exogenous polypeptide, comprising a second cardiovascular therapeutic.

The disclosure also provides, in some aspects, an enucleated erythroid cell, comprising a first exogenous polypeptide comprising a first metabolic therapeutic, and a second exogenous polypeptide, comprising a second metabolic therapeutic.

The disclosure also provides, in some aspects, a method of treating a subject having a cardiovascular disease (e.g., hypercholesterolemia or hereditary angioedema), comprising administering to the subject an effective number of the erythroid cells described herein to the subject, thereby treating the cardiovascular disease.

The disclosure also provides, in some aspects, a method of treating a subject having a metabolic disorder (e.g., a metabolic deficiency), comprising administering to the subject an effective number of the erythroid cells described herein to the subject, thereby treating the metabolic disorder (e.g., a metabolic deficiency).

The disclosure also provides, in some aspects, a method of making an erythroid cell described herein, comprising: a) providing an erythroid cell, b) contacting the erythroid cell with nucleic acid encoding a first exogenous protein (e.g., an exogenous protein of any of Tables 1, 2, 3, or 4) and nucleic acid encoding a second exogenous protein (e.g., an exogenous protein of any of Tables 1, 2, 3, or 4), under conditions that allow uptake of the nucleic acid by the erythroid cell, and c) culturing the cell under conditions that allow for expression of the first and second exogenous proteins, thereby making the erythroid cell.

The disclosure also provides, in some aspects, a plurality of erythroid cells described herein, e.g., wherein the plurality comprises at least 10⁵, 10⁶, 10⁷, 10⁸, 10⁹, 10¹⁰, 10¹¹, or 10¹² erythroid cells according to any of the proceeding claims.

The disclosure also provides, in some aspects, a pharmaceutical composition comprising a cell or plurality of cells described herein.

In some aspects, the present disclosure provides an enucleated erythroid cell, comprising a first exogenous polypeptide comprising a first cardiovascular therapeutic, and a second exogenous polypeptide, comprising a second cardiovascular therapeutic, wherein the first cardiovascular therapeutic is chosen from a natriuretic peptide (e.g., brain natriuretic peptide (BNP)) and a biomarker of cardiac inflammation (e.g., myeloperoxidase (MPO)).

In some aspects, the present disclosure provides a method of treating a subject having a cardiovascular disease, comprising administering to the subject an effective number of erythroid cells which comprise a first exogenous polypeptide comprising a first cardiovascular therapeutic, and a second exogenous polypeptide, comprising a second cardiovascular therapeutic to the subject,

wherein the cardiovascular disease is a cardiovascular disease described herein, e.g., heart failure, atherosclerosis, and thromboembolism,

thereby treating the cardiovascular disease.

In some aspects, the present disclosure provides an enucleated erythroid cell, comprising a first exogenous polypeptide comprising a first metabolic therapeutic, and a second exogenous polypeptide, comprising a second metabolic therapeutic,

wherein the first metabolic therapeutic is chosen from an agent that binds a pancreatic beta cell receptor (e.g., GLP-1), a fibroblast growth factor (e.g., fibroblast growth factor 21 (FGF-21)), leptin, and an agent that targets an osteoclast receptor (e.g., a RANK-L antibody (e.g., (denusomab)).

In some aspects, the present disclosure provides a method of treating a subject having a metabolic disorder (e.g., a metabolic deficiency), comprising administering to the subject an effective number of erythroid cells which comprise a first exogenous polypeptide comprising a first metabolic therapeutic, and a second exogenous polypeptide, comprising a second metabolic therapeutic to the subject,

wherein the metabolic disorder is a metabolic disorder described herein, e.g., diabetes (e.g., type 1 diabetes, type 2 diabetes, or gestational diabetes), insulin insensitivity, and obesity, thereby treating the metabolic disorder.

Any of the aspects herein, e.g., the aspects above, can be characterized by one or more of the embodiments herein, e.g., the embodiments below.

In embodiments, the first and second exogenous polypeptides have agent-additive, agent-synergistic, multiplicative, independent function, localization-based, proximity-dependent, scaffold-based, multimer-based, pathway-based, or compensatory activity.

In embodiments, the enucleated erythroid cell further comprises a third exogenous polypeptide that comprises a third cardiovascular therapeutic.

In embodiments, the enucleated erythroid cell further comprises a third exogenous polypeptide that comprises a third metabolic therapeutic.

In embodiments, one or more of (e.g., 2, 3, 4, 5, 10, or more of):
a) the first and second exogenous polypeptides act on the same target, e.g., a clotting factor, wherein optionally the target is a cell surface receptor and/or an endogenous human protein;
b) the first exogenous polypeptide binds to a first endogenous human protein and the second exogenous polypeptide binds to a second endogenous human target protein, e.g., with a Kd of less than 500, 200, 100, 50, 20, 10, 5, 2, or 1 nM;
c) the first exogenous polypeptide (e.g., an enzyme) acts on (e.g., binds) a first target, and the second exogenous polypeptide (e.g., an enzyme) act on (e.g., binds) a second target, wherein the first and second targets are members of the same biological pathway, wherein optionally the targets are cell surface receptors, endogenous human proteins (e.g., enzymes), or both;
d) the first and second exogenous polypeptides are in close proximity to each other, e.g., are less than 10, 7, 5, 4, 3, 2, 1, 0.5, 0.2, or 0.1 nm apart for a duration of at least 1, 2, 5, 10, 30, or 60 seconds; 1, 2, 5, 10, 30, or 60 minutes, or 1, 2, 3, 6, 12, or 14 hours;
e) the first and second exogenous polypeptides have a Kd of less than 500, 200, 100, 50, 20, 10, 5, 2, or 1 nM for each other;
f) the first and second exogenous polypeptides act on different targets (e.g., clotting factors), wherein optionally at least one of the targets is a cell surface receptor and/or an endogenous human protein, e.g., the first exogenous polypeptide binds a first cell type e.g., an immune effector cell, and the second exogenous polypeptide binds a second cell type, e.g., an immune effector cell, e.g., a T cell;
g) the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number;
h) the first exogenous polypeptide and the second exogenous polypeptide have a Kd for a first target and a second target, respectively, with a ratio of about 1:1, from about 2:1 to 1:2, from about 5:1 to 1:5, from about 10:1 to 1:10, from about 20:1 to 1:20, from about 50:1 to 1:50, from about 100:1 to 1:100;
i) the first exogenous polypeptide has a first activity (e.g., binding) towards a first target, and the second exogenous polypeptide has a second activity (e.g., binding) towards the first target, e.g., the first and second exogenous polypeptides bind a single target;
j) the first exogenous polypeptide acts on (e.g., binds) a first target and the second exogenous polypeptide acts on (e.g., binds) a second target, and the first and second targets are part of the same pathway, wherein optionally the first exogenous polypeptide acts on the first target and the second exogenous polypeptide acts on the second target simultaneously;
k) the first exogenous polypeptide acts on (e.g., binds) a first target and the second exogenous polypeptide acts on (e.g., binds) a second target, and the first and second targets are part of different pathways, wherein optionally the first and second pathways both act to promote a given cellular response;
l) the first exogenous polypeptide localizes the enucleated erythroid cell to a desired site, e.g., a blood clot, and the second exogenous polypeptide has a therapeutic activity, e.g., a fibrinolytic enzyme;
m) the first exogenous polypeptide binds a first cell, e.g., a first cell type, and the second exogenous polypeptide binds a second cell, e.g., a second cell type, e.g., an immune effector cell, e.g., a T cell;
n) the first exogenous polypeptide and the second exogenous polypeptide are non-human proteins, e.g., the enzymes are not natively found in humans;
o) the first exogenous polypeptide and the second exogenous polypeptide are both enzymes, e.g., biosynthetic enzymes;
p) the first exogenous polypeptide (e.g., an enzyme) promotes formation of an intermediate molecule (e.g., converts a substrate into an intermediate) and the second exogenous polypeptide (e.g., an enzyme) acts on the intermediate molecule (e.g., converts an intermediate into a product);
q) the first exogenous polypeptide and the second exogenous polypeptide act on successive steps of a pathway;
r) the erythroid cell comprises at least at least 10 copies, 100 copies, 1,000 copies, 5,000 copies 10,000 copies, 25,000 copies, 50,000 copies, or 100,000 copies of each of the first exogenous polypeptide and the second exogenous polypeptide;
s) the copy number of the first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the second exogenous polypeptide; or
t) the copy number of the second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the first exogenous polypeptide.

In embodiments, the first exogenous polypeptide comprises an anti-PCSK9 antibody molecule or a kallikrein inhibitor (e.g., ecallantaide) or a fragment or variant thereof. In embodiments, the first exogenous polypeptide and the second exogenous polypeptide are enzymes (e.g., enzymes that are not natively found in humans). In embodiments, the first exogenous polypeptide comprises phenylalanine ammonia lyase (PAL) or a phenylalanine-metabolizing fragment or variant thereof, wherein optionally the second exogenous polypeptide comprises an enzyme.

In embodiments, the first exogenous polypeptide acts on (e.g., binds) a clotting factor (e.g., the first exogenous polypeptide is an antibody for the target) and the second exogenous polypeptide activates or inactivates (e.g., cleaves) the clotting factor (e.g., Tissue Factor, Factor V, Factor VII, Factor VIII, Factor IX, Factor X, Factor XI, Factor XIII, thrombin, or fibrinogen). In embodiments, the second exogenous polypeptide comprises a clotting factor that acts on a target (e.g., a substrate), wherein optionally the second exogenous polypeptide comprises Tissue Factor and the target comprises Factor VII; the second exogenous polypeptide comprises TF-VIIa and/or Factor IXa and the target comprises Factor X; the second exogenous polypeptide comprises Factor XIa and the target comprises Factor IX; the second exogenous polypeptide comprises TF-VIIa and the target comprises Factor IX; the second exogenous polypeptide comprises Factor VIIIa and the target comprises Factor X; the second exogenous polypeptide comprises Factor XI; Factor VIII, or Factor V and the target comprises thrombin; the second exogenous polypeptide comprises Factor Va or Xa and the target comprises prothrombin; the second exogenous polypeptide comprises thrombin and the target comprises fibrinogen or Factor XIII; or the second exogenous polypeptide comprises a plasminogen activator (e.g., urokinase or tissue plasminogen activator (TPA)) and the target comprises plasminogen. In embodiments, the first exogenous polypeptide acts on (e.g., binds) a blood clot (e.g., the first exogenous polypeptide comprises an anti-fibrin antibody molecule, fibrin, or a fibrin-binding portion or variant thereof) and the second exogenous polypeptide is a fibrinolytic enzyme (e.g., plasmin or a fibrinolytic fragment or variant thereof).

In embodiments, the metabolic disorder (e.g., a metabolic deficiency) is selected from the group consisting of hemophilia (e.g., hemophilia type A, hemophilia type B, or hemophilia type C), von Willebrand disease, Factor II deficiency, Factor V deficiency, Factor VII deficiency, Factor X deficiency, Factor XII deficiency, thrombotic thrombocytopenic purpura, Phenylketonuria (PKU), Adenosine Deaminase Deficiency-Severe Combined Immunodeficiency (ADA-SCID), Mitochondrial Neurogastrointestinal Encephalopathy (MNGIE), Primary Hyperoxaluria, Alkaptonuria, and Thrombotic Thrombocytopenic Purpura (TTP).

In embodiments, the first exogenous polypeptide acts on (e.g., binds) a clotting factor and the second exogenous polypeptide activates or inactivates (e.g., cleaves) the clotting factor (e.g., Tissue Factor, Factor V, Factor VII, Factor VIII, Factor IX, Factor X, Factor XI, Factor XIII, thrombin, or fibrinogen), wherein optionally the second exogenous polypeptide comprises an activated clotting factor (e.g., Tissue Factor, Factor V, Factor VII, Factor VIII, Factor IX, Factor X, Factor XI, Factor XIII, thrombin, or fibrinogen). In embodiments, the subject has a clotting deficiency disease such as hemophilia (e.g., hemophilia type A, hemophilia type B, or hemophilia type C), von Willebrand disease, Factor II deficiency, Factor V deficiency, Factor VII deficiency, Factor X deficiency, or Factor XII deficiency. In embodiments, the second exogenous polypeptide comprises a clotting factor that acts on a target, wherein optionally the second exogenous polypeptide comprises Tissue Factor and the target comprises Factor VII; the second exogenous polypeptide comprises TF-VIIa or Factor IXa and the target comprises Factor X; the second exogenous polypeptide comprises Factor XIa and the target comprises Factor IX; the second exogenous polypeptide comprises TF-VIIa and the target comprises Factor IX; the second exogenous polypeptide comprises Factor VIIIa and the target comprises Factor X; the second exogenous polypeptide comprises Factor XI; Factor VIII, or Factor V and the target comprises thrombin (e.g., Factor IIa); the second exogenous polypeptide comprises Factor Va or Xa and the target comprises prothrombin; the second exogenous polypeptide comprises thrombin (e.g., Factor IIa) and the target comprises fibrinogen or Factor XIII; or the second exogenous polypeptide comprises a plasminogen activator (e.g., urokinase or tissue plasminogen activator (TPA)) and the target comprises plasminogen. In embodiments, the second exogenous polypeptide reduces unwanted clotting in a subject having or at risk of developing a blood clot such as the subject has, or is at risk of developing, thrombophilia, pulmonary embolism, or stroke. In embodiments, the metabolic disorder (e.g., a metabolic deficiency) is hemophilia A, the second exogenous polypeptide comprises Factor VIII or fragment thereof, and the target is thrombin (e.g., Factor Ha) or Factor X. In embodiments, the metabolic disorder (e.g., a metabolic deficiency) is hemophilia B, the first exogenous polypeptide binds Factor XIa or factor X or a fragment thereof (e.g., is an antibody for Factor XIa or Factor X or a fragment thereof), and the second exogenous polypeptide comprises factor IX or fragment thereof.

In embodiments, the metabolic disorder (e.g., a metabolic deficiency) is thrombotic thrombocytopenic purpura, the first exogenous polypeptide binds ultra-large von Willebrand factor (ULVWF) or fragment thereof (e.g., the first exogenous polypeptide is an antibody for ULVWF), and the second exogenous polypeptide comprises ADAMTS13 or fragment thereof.

In embodiments, the first exogenous polypeptide acts on (e.g., binds) a blood clot and the second exogenous polypeptide comprises a fibrinolytic enzyme (e.g., plasmin or a fibrinolytic fragment or variant thereof), wherein optionally the first exogenous polypeptide is selected from the group consisting of an anti-fibrin antibody molecule, fibrin, and a fibrin-binding portion or variant thereof. In embodiments, the metabolic disorder (e.g., a metabolic deficiency) is PKU, the first exogenous polypeptide comprises phenylalanine ammonia lyase (PAL) or a phenylalanine-metabolizing fragment or variant thereof, and the second exogenous polypeptide comprises an enzyme. In embodiments, the metabolic disorder (e.g., a metabolic deficiency) is ADA-SCID and the first exogenous polypeptide comprises adenosine deaminase (ADA) or a fragment or variant thereof. In embodiments, the metabolic disorder (e.g., a metabolic deficiency) is Mitochondrial Neurogastrointestinal Encephalopathy and the first exogenous polypeptide comprises thymidine phosphorylase or a fragment or variant thereof. In embodiments, the metabolic disorder (e.g., a metabolic deficiency) is Primary Hyperoxaluria and the first exogenous polypeptide comprises oxalate oxidase or a fragment or variant thereof. In embodiments, the metabolic disorder (e.g., a metabolic deficiency) is Alkaptonuria and the first exogenous polypeptide comprises homogentisate oxidase or a fragment or variant thereof. In embodiments, the metabolic disorder (e.g., a metabolic deficiency) is Thrombotic Thrombocytopenic Purpura and the first exogenous polypeptide comprises ADAMTS13 or a fragment or variant thereof.

In embodiments, the first exogenous polypeptide binds to a target more strongly than the first exogenous polypeptide binds to the second exogenous polypeptide.

In embodiments, the first exogenous polypeptide promotes fusion of the erythroid cell with a target cell.

In embodiments, e.g., embodiments of methods of making erythroid cells described herein, the nucleic acid encoding the first exogenous protein and the nucleic acid encoding the second exogenous protein are separate nucleic acids. In embodiments, the nucleic acid encoding the first exogenous protein and the nucleic acid encoding the second exogenous protein are part of the same nucleic acid molecule.

In some embodiments,

the first cardiovascular therapeutic comprises a naturetic peptide (e.g., BNP) and the second cardiovascular therapeutic comprises relaxin (e.g., relaxin 2), e.g., for the treatment of heart failure;

the first cardiovascular therapeutic comprises a biomarker of cardiac inflammation (e.g., myeloperoxidase (MPO)) and the second cardiovascular therapeutic comprises relaxin (e.g., relaxin 2), e.g., for the treatment of heart failure;

the first cardiovascular therapeutic comprises a plasminogen activator (e.g., tissue plasminogen activator (TPA)) and the second cardiovascular therapeutic comprises a tissue factor pathway inhibitor (TFPI) , e.g., for the treatment of atherosclerosis or thromboembolism.

In some embodiments, the first metabolic therapeutic comprises an agent that binds a pancreatic beta cell receptor (e.g., GLP-1) and the second metabolic therapeutic comprises an agent that regulates glucose metabolism (e.g., insulin), e.g., for the treatment of diabetes (e.g., type 1 diabetes, type 2 diabetes, or gestational diabetes) and/or insulin insensitivity; the first metabolic therapeutic comprises a fibroblast growth factor (e.g., fibroblast growth factor 21 (FGF-21)) and the second metabolic therapeutic comprises an agent that binds a glucagon receptor (e.g., a secretin, e.g., glucagon) , e.g., for the treatment of obesity;

the first metabolic therapeutic comprises leptin and the second metabolic therapeutic comprises an agent that binds a glucagon receptor (e.g., a secretin, e.g., glucagon) , e.g., for the treatment of obesity;

the first metabolic therapeutic comprises an agent that targets an osteoclast receptor e.g., RANK-L (e.g., a RANK-L antibody, e.g., denusomab) and the second metabolic therapeutic comprises an osteoclast activator, e.g., a ligand for parathyroid hormone 1 receptor, e.g., parathyroid hormone (PTH), e.g., for the treatment of osteoporosis;

the first metabolic therapeutic comprises a cystathionine B-synthase (CBS) polypeptide and the second metabolic therapeutic comprises an L-homocysteine or L-serine transporter, e.g., sodium-coupled neutral amino acid transporter 2 (SAT2) or neutral amino acid transporter A (ASCT1), e.g., for the treatment of homocystinuria; or

the first metabolic therapeutic comprises an uricase polypeptide and the second metabolic therapeutic comprises a catalase polypeptide , e.g., for the treatment of hyperuricemia.

In some embodiments, the first exogenous polypeptide (e.g., a GLP-1 polypeptide) increases the subject's sensitivity to the second exogenous polypeptide (e.g., an insulin polypeptide). In some embodiments, the first exogenous polypeptide comprises a GLP-1 polypeptide and the second exogenous polypeptide comprises an agent that extends GLP-1 half-life, e.g., a DPP-4 inhibitor. In embodiments, the subject (e.g., a subject treated with erythroid cells described herein, e.g., erythroid cells comprising a leptin polypeptide and optionally further comprising a glucagon peptide) has resistance to leptin, e.g., has a leptin receptor mutation. In embodiments, the cardiovascular therapeutic comprises an anti-coagulation agent such as antithrombin (e.g., GenBank Accession number NP_000479, or an active fragment or variant thereof). In embodiments, the cardiovascular therapeutic comprises a fibrinolytic enzyme, e.g., plasmin (e.g., GenBank Accession number NP_000292, or an active fragment or variant thereof).

In embodiments, the exogenous polypeptide comprises an incretin, e.g., GLP-1. In embodiments, a subject (e.g., a diabetic subject) treated with an erythroid cell comprising GLP-1 exhibits a reduction in one or more symptoms of a metabolic disease, e.g., exhibits weight loss (e.g., as assessed by an improvement in body mass index (BMI)), a decrease in hypoglycemia (e.g., in frequency or severity of hypoglycemic events), increased energy, decreased hunger, decreased plasma glucose, decreased plasma triglycerides, or improved insulin sensitivity.

In some embodiments, one of the exogenous polypeptides comprises phenylalanine ammonia lyase (PAL) or a phenylalanine-metabolizing fragment or variant thereof. In some embodiments, the second exogenous polypeptide comprises an enzyme. In embodiments, a subject (e.g., a subject with PKU) treated with an erythroid cell comprising PAL or a phenylalanine-metabolizing fragment or variant thereof exhibits an improvement in one or more symptoms of PKU, e.g., an improvement in mental retardation, epilepsy, organ damage, or posture.

In embodiments, at least:

(a) and (b), (a) and (c), (a) and (d), (a) and (e), (a) and (f), (a) and (g), (a) and (h), (a) and (i), (a) and (j), (a) and (k), (a) and (l), (a) and (m), (a) and (n), (a) and (o), (a) and (p), (a) and (q), (a) and (r), (a) and (s), (a) and (t),

(b) and (c), (b) and (d), (b) and (e), (b) and (f), (b) and (g), (b) and (h), (b) and (i), (b) and (j), (b) and (k), (b) and (l), (b) and (m), (b) and (n), (b) and (o), (b) and (p), (b) and (q), (b) and (r), (b) and (s), (b) and (t),

(c) and (d), (c) and (e), (c) and (f), (c) and (g), (c) and (h), (c) and (i), (c) and (j), (c) and (k), (c) and (l), (c) and (m), (c) and (n), (c) and (o), (c) and (p), (c) and (q), (c) and (r), (c) and (s), (c) and (t),

(d) and (e), (d) and (f), (d) and (g), (d) and (h), (d) and (i), (d) and (j), (d) and (k), (d) and (l), (d) and (m), (d) and (n), (d) and (o), (d) and (p), (d) and (q), (d) and (r), (d) and (s), (d) and (t),

(e) and (f), (e) and (g), (e) and (h), (e) and (i), (e) and (j), (e) and (k), (e) and (l), (e) and (m), (e) and (n), (e) and (o), (e) and (p), (e) and (q), (e) and (r), (e) and (s), (e) and (t),

(f) and (g), (f) and (h), (f) and (i), (f) and (j), (f) and (k), (f) and (l), (f) and (m), (f) and (n), (f) and (o), (f) and (p), (f) and (q), (f) and (r), (f) and (s), (f) and (t),

(g) and (h), (g) and (i), (g) and (j), (g) and (k), (g) and (l), (g) and (m), (g) and (n), (g) and (o), (g) and (p), (g) and (q), (g) and (r), (g) and (s), (g) and (t),

(h) and (i), (h) and (j), (h) and (k), (h) and (l), (h) and (m), (h) and (n), (h) and (o), (h) and (p), (h) and (q), (h) and (r), (h) and (s), (h) and (t),

(i) and (j), (i) and (k), (i) and (l), (i) and (m), (i) and (n), (i) and (o), (i) and (p), (i) and (q), (i) and (r), (i) and (s), (i) and (t),

(j) and (k), (j) and (l), (j) and (m), (j) and (n), (j) and (o), (j) and (p), (j) and (q), (j) and (r), (j) and (s), (j) and (t),

(k) and (l), (k) and (m), (k) and (n), (k) and (o), (k) and (p), (k) and (q), (k) and (r), (k) and (s), (k) and (t),

(l) and (m), (l) and (n), (l) and (o), (l) and (p), (l) and (q), (l) and (r), (l) and (s), (l) and (t),

(m) and (n), (m) and (o), (m) and (p), (m) and (q), (m) and (r), (m) and (s), (m) and (t),

(n) and (o), (n) and (p), (n) and (q), (n) and (r), (n) and (s), (n) and (t),

(o) and (p), (o) and (q), (o) and (r), (o) and (s), (o) and (t),

(p) and (q), (p) and (r), (p) and (s), (p) and (t),

(q) and (r), (q) and (s), (q) and (t),

(r) and (s), (r) and (t), or

(s) and (t), are present.

In some embodiments, the exogenous polypeptides have synergistic activity. In some embodiments, the exogenous polypeptides have additive activity. In some embodiments, the exogenous polypeptides have proximity-dependent activity. The proximity between the plurality of polypeptides, before, during, or after, interaction with a target moiety or moieties, may confer a property or result which is not seen in the absence of such proximity in vivo or in vitro.

In some embodiments, the first exogenous polypeptide interacts with, e.g., binds, a first target moiety, e.g., a first target cell polypeptide on a target cell (e.g., an immune effector cell, e.g., a T cell), and the second exogenous polypeptide interacts with, e.g., binds, a second target moiety, e.g., a second target cell polypeptide on the target cell (e.g., wherein binding of the first and second target cell polypeptide alters a biological property of the target cell). In an embodiment the first and second targets are subunits of a multimeric complex on the target cell.

In some embodiments, the first exogenous polypeptide promotes fusion of the enucleated erythroid cell with a target cell and the second exogenous polypeptide is a polypeptide of any of Table 1, Table 2, Table 3, or Table 4 (e.g., a human polypeptide of any of Table 1, Table 2, Table 3, or Table 4, e.g., a polypeptide having the amino acid sequence of the human wild type polypeptide).

In some embodiments the first and second exogenous polypeptides interact with one another, e.g., the first modifies, e.g., by cleavage or phosphorylation, the second, or the first and second form a dimeric or multimeric protein.

In some embodiments, the enucleated erythroid cell comprises 3, 4, 5, 6, 7, 8, 9, or 10 different exogenous polypeptides. In an embodiment a plurality (e.g., 2, 3, 4, 5, 6, 7, 8, 9, or 10), or all, of the different exogenous polypeptides, have a preselected level of homology to each other, e.g., at least 40, 50, 60, 70, 75, 80, 85, 90, 95, 96, 97, 98, 99, or 99.5% sequence identity to each other. In an embodiment a plurality (e.g., 2, 3, 4, 5, 6, 7, 8, 9, or 10), or all, of the different exogenous polypeptides, have a preselected level of homology to a reference sequence, e.g., at least 40, 50, 60, 70, 75, 80, 85, 90, 95, 96, 97, 98, 99, 99.5%, or 100% sequence identity with a reference sequence (which reference sequence, includes an entire polypeptide sequence, or a portion thereof, e.g., a preselected domain), e.g., a plurality or all of the different exogenous polypeptides are antibodies or antibody molecules. In some embodiments, the reference sequence is an antibody sequence or fragment thereof. In some embodiments, the reference sequence comprises a heavy chain constant region or portion thereof, light chain constant region or fragment thereof, heavy chain variable region or portion thereof, light chain variable region or fragment thereof, or any combination of the foregoing.

In some embodiments, the enucleated erythroid cell comprises at least 2 but no more than 5, 6, 7, 8, 9, or 10 different exogenous polypeptides, e.g., exogenous polypeptides that are encoded by one or more exogenous nucleic acids that are not retained by the enucleated erythroid cell.

In some embodiments, the exogenous polypeptides are encoded by one or more exogenous nucleic acids that are not retained by the enucleated erythroid cell.

In some embodiments, one or more (e.g., two or three) of the first, second, and optionally third exogenous polypeptides are transmembrane polypeptides or surface-anchored polypeptides. In some embodiments, the first exogenous polypeptide is a transmembrane polypeptides or surface-anchored polypeptide, and the second exogenous polypeptide is internal to the erythroid cell and not associated with the cell membrane. In some embodiments, the second exogenous polypeptide is a transmembrane polypeptides or surface-anchored polypeptide, and the first exogenous polypeptide is internal to the erythroid cell and not associated with the cell membrane. In some embodiments, both of the first and second exogenous polypeptides are internal to the erythroid cell and not associated with the cell membrane.

In some embodiments, the first exogenous polypeptide interacts with, e.g., binds, a moiety on a target cell, and the second exogenous polypeptide alters a property of the target cell, e.g., down regulates inflammation in the target cell.

In some embodiments, the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, or from about 100:1 to 1:100 by weight or by copy number. In some embodiments, both the first and second polypeptides have a stoichiometric mode of action, or both have a catalytic mode of action, and both are present at a similar abundance, e.g., about 1:1 or from about 2:1 to 1:2. In some embodiments, the first exogenous polypeptide is more abundant than the second exogenous polypeptide by at least about 10%, 20%, 30%, 50%, or a factor of 2, 3, 4, 5, 10, 20, 50, or 100 (and optionally up to 10 or 100 fold) by weight or copy number. In some embodiments, the second exogenous polypeptide is more abundant than the first exogenous polypeptide by at least about 10%, 20%, 30%, 50%, or a factor of 2, 3, 4, 5, 10, 20, 50, or 100 (and optionally up to 10 or 100 fold) by weight or copy number. In some embodiments, the first polypeptide has a stoichiometric mode of action and the second polypeptide has a catalytic mode of action, and the first polypeptide is more abundant than the second polypeptide. In some embodiments, the second polypeptide has a stoichiometric mode of action and the first polypeptide has a catalytic mode of action, and the second polypeptide is more abundant than the first polypeptide.

In some embodiments, the first exogenous polypeptide comprises a targeting moiety.

In some embodiments, the enucleated erythroid cell has one or more of the following characteristics:
a) an osmotic fragility of less than 50% cell lysis at 0.3%, 0.35%, 0.4%, 0.45%, or 0.5% NaCl;
b) a cell volume of about 10-200 fL or a cell diameter of between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns, or between about 10 microns and about 30 microns;
c) greater than 1%, 2%, 3%, 4%, 5%, 6%, 7%, 8%, 9% or 10% fetal hemoglobin; or at least about 20, 25, or 30 pg/cell of hemoglobin; or
d) phosphatidylserine content of the outer leaflet is less than 30%, 25%, 20%, 15%, 10%, or 5% as measured by Annexin V staining.

In some embodiments, at least one, e.g., all, of the plurality of exogenous polypeptides are glycosylated. In some embodiments, at least one, e.g., all, of the plurality of exogenous polypeptides are phosphorylated.

In some embodiments, the enucleated erythroid cell is a reticulocyte.

In some embodiments, the exogenous polypeptide or polypeptides lack a sortase transfer signature (i.e., a sequence that can be created by a sortase reaction) such as LPXTG (SEQ ID NO: 32).

In some embodiments, the enucleated erythroid cell comprises:

a first exogenous polypeptide that interacts with a target, and

a second exogenous polypeptide that modifies the target.

In some embodiments, one or more of:

(a) the second exogenous polypeptide comprises an enzyme (e.g., a protease) that modifies, e.g., is specific, e.g., binds to a site on target, binds (e.g., specifically) and modifies, e.g., covalently modifies, e.g., cleaves, or removes or attaches a moiety to, the target, wherein the target is optionally a complement factor or a pro-inflammatory cytokine;

(b) the second exogenous polypeptide comprises a polypeptide, e.g., an enzyme, e.g., a protease, that modifies the secondary, tertiary, or quaternary structure of the target, and, in embodiments, alters, e.g., decreases or increases, the ability of the target to interact with another molecule, e.g., the first exogenous polypeptide or a molecule other than the first exogenous polypeptide, wherein optionally the target comprises a pro-inflammatory cytokine, or complement factor;

(c) the second exogenous polypeptide comprises a polypeptide, e.g., an enzyme (e.g., a protease) that cleaves the target, e.g., a polypeptide, between a first target domain and a second target domain, e.g., a first target domain that binds a first substrate and a second target domain that binds a second substrate;

(d) the target is a polypeptide (e.g., a pro-inflammatory cytokine or a complement factor); a carbohydrate (e.g., a glycan), a lipid (e.g., a phospholipid), or a nucleic acid (e.g., DNA, or RNA);

(e) the first exogenous polypeptide binds but does not cleave a target and the second exogenous polypeptide cleaves a bond e.g., a covalent bond, e.g., a covalent bond in the target;

(f) the first exogenous polypeptide has an affinity for the target that is about 1-2 pM, 2-5 pM, 5-10 pM, 10-20 pM, 20-50 pM, 50-100 pM, 100-200 pM, 200-500 pM, 500-1000 pM, 1-2 nM, 2-5 nM, 5-10 nM, 10-20 nM, 20-50 nM, 50-100 nM, 100-200 nM, 200-500 nM, 500-1000 nM, 1-2 μM, 2-5 μM, 5-10 μM, 10-20 μM, 20-50 μM, or 50-100 μM;

(g) the second exogenous polypeptide has a K_{M} for the target of about 10⁻¹-10⁻⁷M, 10⁻¹-10⁻²M, 10⁻²-10⁻³M, 10⁻³-10⁴M, 10⁻⁴-10⁻⁵M, 10⁻⁵-10⁻⁶M, or 10⁻⁶-10⁻⁷M;

(h) a ratio of the K_{d} of the first exogenous polypeptide for the target (measured in M) divided by the K_{M} of the second exogenous polypeptide for the target (measured in M) is about 1×10⁻⁹-2×10⁻⁹, 2×10⁻⁹-5×10⁻⁹, 5×10⁻⁹-1×10⁻⁸, 1×10⁻⁸-2×10⁻⁸, 2×10⁻⁸-5×10⁻⁸, 5×10⁻⁸-×10⁻⁷, 1×10⁻⁷-2×10⁻⁷, 2×10⁻⁷-5×10⁻⁷, 5×10⁻⁷ -1×10⁻⁶, 1×10⁻⁶-2×10⁻⁶, 2×10⁻⁶-5×10⁻⁶, 5×10⁻⁶-1×10⁻⁵, 1×10⁻⁵-2×10⁻⁵, 2×10⁻⁵-5×10⁻⁵, 5×10⁻⁵-1×10⁻⁴, 1×10-2×10⁻⁴, 2×10-5×10⁻⁴, 5×10⁻⁴-1×10³, 1×10³-2×10³, 2×10³-5×10³, 5×10³-1×10², 1×10²-2×10², 2×10⁻²-5×10⁻², 5×10⁻²-1×10⁻¹, 1×10⁻¹-2×10⁻¹, 2×10⁻¹-5×10⁻¹, or 5×10⁻¹-1;

(i) the observed reaction rate of the second exogenous polypeptide modifying the target is greater than the reaction rate of an enucleated cell which is similar but which lacks the first exogenous polypeptide under otherwise similar reaction conditions;

(j) a ratio of the average number of the first exogenous polypeptide on the erythroid cell to the average number of the second exogenous polypeptide on the erythroid cell is about 50:1, 20:1, 10:1, 8:1, 6:1, 4:1, 2:1, 1:1, 1:2, 1:4, 1:6, 1:8, 1:10, 1:20, or 1:50;

(k) affinity of the first exogenous polypeptide for the target is greater than the affinity of the first exogenous polypeptide for the modified (e.g., cleaved) target;

(l) a therapeutically effective dose of the enucleated erythroid cell is less than stoichiometry (e.g., less by 10%, 20%, 30%, 40%, 50%, 60%, 70%, 80%, 90%, 95%, 98%, 99%, 99.5%, 99.9%, or 99.99%) to the amount of target in a subject's peripheral blood at the time of administration;

(m) the number of enucleated erythroid cells in an effective dose, is less than (e.g., less by 10%, 20%, 30%, 40%, 50%, 60%, 70%, 80%, 90%, 95%, 98%, 99%, 99.5%, 99.9%, or 99.99%) the number of targets, e.g., target molecules, in the subject's peripheral blood at the time of administration;

(n) the number of second exogenous polypeptides comprised by a preselected amount of enucleated erythroid cells, e.g., an effective dose, or in vitro effective amount of enucleated erythroid cells, is less than (e.g., less by 10%, 20%, 30%, 40%, 50%, 60%, 70%, 80%, 90%, 95%, 98%, 99%, 99.5%, 99.9%, or 99.99%) a reference value for targets, e.g., less than the number of targets in the peripheral blood of the subject at the time of administration;

(o) the number of first exogenous polypeptides comprised by a preselected amount of enucleated erythroid cells, e.g., an effective dose, or in vitro effective amount of enucleated erythroid cells, is less than (e.g., less by 10%, 20%, 30%, 40%, 50%, 60%, 70%, 80%, 90%, 95%, 98%, 99%, 99.5%, 99.9%, or 99.99%) a reference value for targets, e.g., less than the number of targets in the peripheral blood of the subject at the time of administration;

(p) the number of first exogenous polypeptides and the number of second exogenous polypeptides comprised by a preselected amount of enucleated erythroid cells, e.g., an effective dose, enucleated erythroid cells, is each less than a reference value for targets, e.g., less than the number of targets in the peripheral blood of the subject at the time of administration;

(q) the second exogenous polypeptide modifies (e.g. cleaves) the target with a K_{M} of at least 10⁻¹ M, 10⁻²M, 10⁻³ M, 10⁻⁴ M, 10⁻⁵ M, 10⁻⁶M, or 10⁻⁷ M;

(r) the second exogenous polypeptide comprises a chaperone;

(s) the first exogenous polypeptide comprises a surface-exposed portion and the second exogenous polypeptide comprises a surface exposed portion; or

(t) an effective amount of the enucleated erythroid cells is less than (e.g., less by 10%, 20%, 30%, 40%, 50%, 60%, 70%, 80%, 90%, 95%, 98%, 99%, 99.5%, 99.9%, or 99.99%) an effective amount of otherwise similar enucleated erythroid cells that lack the second exogenous polypeptide.

In embodiments, (a) the second exogenous polypeptide comprises an enzyme (e.g., a protease) that modifies, e.g., is specific, e.g., binds to a site on target, binds (e.g., specifically) and modifies, e.g., covalently modifies, e.g., cleaves, or removes or attaches a moiety to, the target, wherein the target is optionally a pro-inflammatory cytokine or a complement factor. In embodiments the modification alters, e.g., increases or decreases, the ability of the target to interact with another molecule, e.g., the first exogenous polypeptide or a molecule other than the first exogenous polypeptide.

In embodiments, (c) the second exogenous polypeptide comprises a polypeptide, e.g., an enzyme (e.g., a protease) that cleaves the target, e.g., a polypeptide, between a first target domain and a second target domain, e.g., a first target domain that binds a first substrate and a second target domain that binds a second substrate. In embodiments the first target domain is released from the second target domain. In embodiments cleavage alters the affinity one or both of the first target domain for a first substrate and the affinity of the second target domain for a second substrate.

In embodiments, the target is other than an infectious component, e.g., other than a bacterial component, a viral component, a fungal component, or a parasitic component. In embodiments, the surface-exposed portion of the first exogenous polypeptide binds the target. In embodiments, the surface-exposed portion of the second exogenous polypeptide comprises enzymatic activity, e.g., protease activity. In embodiments, the surface-exposed portion of the second exogenous polypeptide enzymatically modifies, e.g., cleaves, the target In embodiments, the enucleated erythroid cell is capable of clearing the target from a subject's body at a faster rate than an otherwise similar enucleated erythroid cell that lacks the second exogenous polypeptide.

In embodiments, the enucleated erythroid cell is complexed with the target or a reaction product of the second exogenous protein acting on the target, e.g., during cleavage.

In some embodiments of any of the compositions and methods described herein

i) at least 50, 60, 70, 80, 90, 95, or 99% of the exogenous polypeptides, e.g., fusion proteins on the surface of the erythroid cell have an identical sequence,

ii) at least 50, 60, 70, 80, 90, 95, or 99% of the exogenous polypeptides, e.g., fusion proteins have the same transmembrane region,

iii) the first and/or second exogenous polypeptide, e.g., fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;

iv) at least 50, 60, 70, 80, 90, 95, or 99% of the exogenous polypeptides, e.g., fusion proteins do not differ from one another by more than 1, 2, 3, 4, 5, 10, 20, or 50 amino acids,

v) the first and/or second exogenous polypeptide lacks a sortase transfer signature,

vi) the first and/or second exogenous polypeptide comprises a moiety that is present on less than 1, 2, 3, 4, or 5 sequence distinct fusion polypeptides;

vii) the first and/or second exogenous polypeptide is present as a single fusion polypeptide;

viii) the first and/or second exogenous polypeptide, e.g., fusion protein does not contain Gly-Gly at the junction of an endogenous transmembrane protein and the moiety;

ix) the first and/or second exogenous polypeptide, e.g., fusion protein does not contain Gly-Gly, or the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment; or a combination thereof.

In some aspects, the present disclosure provides a method of treating a disease or condition described herein, comprising administering to a subject in need thereof an enucleated erythroid cell, e.g., a reticulocyte, described herein. In some embodiments, the disease or condition is a cardiovascular disease or a metabolic disease.

In some aspects, the present disclosure provides a method of bringing into proximity a first and a second cell surface moiety, e.g., transmembrane receptors, e.g., endogenous receptors of a subject having a cardiovascular disease or metabolic disease, comprising administering to a subject in need thereof an enucleated erythroid cell, e.g., a reticulocyte, described herein.

In some aspects, the present disclosure provides a method of delivering, presenting, or expressing a plurality of proximity-dependent molecules comprising providing an enucleated erythroid cell, e.g., a reticulocyte, described herein.

In some aspects, the present disclosure provides a method of producing an enucleated erythroid cell, e.g., a reticulocyte, described herein, providing contacting an erythroid cell precursor with one or more nucleic acids encoding the exogenous polypeptides and placing the cell in conditions that allow enucleation to occur.

In some aspects, the present disclosure provides a preparation, e.g., pharmaceutical preparation, comprising a plurality of enucleated erythroid cells, e.g., reticulocytes, described herein, e.g., at least 10⁸, 10⁹, 10¹⁰, 10¹¹, or 10¹² cells.

In some aspects, the present disclosure provides a cell complex, e.g., an in vitro or in vivo complex, of an engineered erythroid cell, e.g., an enucleated erythroid cell, e.g., a reticulocyte, and a target cell, the complex mediated by one of the exogenous polypeptides. In some embodiments, the cell complex comprises at least 2, 3, 4, 5, 10, 20, 50, or 100 cells.

In some aspects, the present disclosure proves a reaction mixture comprising an engineered erythroid cell, e.g., an enucleated erythroid cell, e.g., a reticulocyte, and nucleic acid, e.g., one or more nucleic acid molecules, encoding a multimodal pair described herein. In some embodiments, the nucleic acid comprises at least one promoter that is active in an erythroid cell. In some embodiments, nucleic acid encodes at least two proteins described herein (e.g., in Table 1, Table 2, Table 3, and Table 4). In some embodiments, the nucleic acid encodes a third exogenous polypeptide.

In some aspects, the present disclosure comprises a method of making an engineered erythroid cell (e.g., an enucleated erythroid cell, e.g., a reticulocyte) described herein, comprising: providing, e.g., receiving, information about a target cell or subject, responsive to that information selecting a plurality of exogenous polypeptides, and introducing nucleic acids encoding the exogenous polypeptides into an erythroid cell or erythroid cell precursor.

In some aspects, the present invention comprises a method of evaluating an engineered erythroid cell (e.g., enucleated erythroid cell, e.g., a reticulocyte), comprising providing a candidate erythroid cell, and determining if nucleic acid encoding a plurality of exogenous polypeptides, e.g., a multimodal pair of the exogenous polypeptides, are present.

The present disclosure also provides, in some aspects, a nucleic acid composition comprising:

a first nucleic acid sequence encoding a first exogenous polypeptide that interacts with a target, e.g., a first exogenous polypeptide described herein,

a second nucleic acid sequence encoding a second exogenous polypeptide that modifies the target, e.g., a second nucleic acid sequence described herein and

optionally, a promoter sequence that is active in an erythroid cell.

In embodiments, the first nucleic acid sequence and second nucleic acid sequence are contiguous or are separate molecules (e.g., admixed molecules or in separate containers). In embodiments, the first nucleic acid sequence and second nucleic acid sequence are part of the same open reading frame and have a protease cleavage site situated therebetween. In embodiments, the first nucleic acid is operatively linked to a first promoter and the second nucleic acid is operatively linked to a second promoter.

The disclosure provides, in some aspects, a kit comprising:

(A) nucleic acids encoding: (A-i) a plurality of binding moieties (e.g., antibody molecules, e.g., scFv domains), fused to (A-ii) a membrane anchor domain, e.g., a transmembrane domain, wherein (A-i) and (A-ii) are operatively linked to a nucleic acid that directs expression in an erythroid cell; and

(B) nucleic acids encoding (B-i) a plurality of enzymes (e.g., proteases), optionally fused to (B-ii) a membrane anchor domain, e.g., a transmembrane domain, wherein (B-i) and (B-ii) are operatively linked to nucleic acid that directs expression in an erythroid cell.

The present disclosure provides, in some aspects, a method of making a fragment of a target, e.g., a clotting factor or a blood clot, comprising contacting the target polypeptide with an erythroid cell described herein. In embodiments, the second exogenous polypeptide cleaves the target to provide the fragment. In embodiments, the contacting comprises administering the erythroid cell to a subject that comprises the target polypeptide.

The present disclosure also provides, in some aspects, a method of converting or activating a target, e.g., a clotting factor, e.g., converting a prodrug to a drug, comprising contacting the polypeptide with an erythroid cell described herein. In embodiments, the second exogenous polypeptide interacts with the target (e.g., prodrug), e.g., cleaves the target. In embodiments, the contacting comprises administering the erythroid cell to a subject that comprises the polypeptide, e.g., prodrug. In embodiments, the subject has hemophilia.

The present disclosure also provides, in some aspects, a method of converting an endogenous polypeptide from a first activity state to a second activity state (e.g., from an inactive state to an active state or an active state to an inactive state), comprising contacting the endogenous polypeptide with an erythroid cell described herein. In embodiments, the second exogenous polypeptide interacts with the target, e.g., covalently modifies, e.g., cleaves the target, or alters its ability to interact with, e.g., bind, one or more other molecules. In embodiments, the target is a clotting factor, e.g., an inactive clotting factor. In embodiments, the second exogenous polypeptide is a clotting factor, e.g., an active clotting factor. In embodiments, the target is a metabolite, e.g., amino acid. In embodiments, the contacting comprises administering the erythroid cell to a subject that comprises the endogenous polypeptide.

The disclosure provides, in some aspects, a method of reducing a level of a target (e.g., a metabolite, amino acid, clotting factor, blood clot protein, pro-inflammatory cytokine, or complement factor) in a subject, comprising administering to the subject an erythroid cell described herein. In embodiments, the second exogenous polypeptide interacts with the target, e.g., covalently modifies, e.g., cleaves the target, or alters its ability to interact with, e.g., bind, another molecule.

The disclosure contemplates all combinations of any one or more of the foregoing aspects and/or embodiments, as well as combinations with any one or more of the embodiments set forth in the detailed description and examples.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Jul. 19, 2017. When one gene or protein references a plurality of sequence accession numbers, all of the sequence variants are encompassed.

### BRIEF DESCRIPTION OF THE DRAWINGS

FIG. 1 is a set of graphs showing results of a Raji apoptosis assay measured through flow cytometry. Raji cells are CFSE labeled and co-cultured with erythroid differentiated cells that are untransduced (control) and transduced with single or multiple TRAIL variants or co-cultured with two different singly transduced cells. Percent apoptosis determined by percent of cells that are Raji (CFSE+) and annexin V+(Top). Flow cytometry plots of CFSE and annexin V staining of various conditions. (Bottom). Graph of percent apoptosis of the various conditions.

FIG. 2 is a bar graph showing the mean fluorescent intensity from control erythroid cells (UNT) or IdeS-expressing erythroid cells (IDES) labelled with an anti-Rabbit Fc fluorophore labeled antibody, before or after a 5 hour incubation.

FIG. 3 is a Western blot showing intact heavy chain of target antibodies or fragments of the heavy chain in supernatant from control cells (UNT) or Ide-S expressing cells (IdeS-RCT). Arrows indicate the heavy chain (Hc), heavy chain fragment (Hc-fragment), and light chain (Lc).

### DETAILED DESCRIPTION OF THE INVENTION

### Definitions

As used herein, the term “antibody molecule” refers to a protein, e.g., an immunoglobulin chain or fragment thereof, comprising at least one immunoglobulin variable domain sequence.

The term “antibody molecule” encompasses antibodies and antibody fragments. In an embodiment, an antibody molecule is a multispecific antibody molecule, e.g., a bispecific antibody molecule. Examples of antibody molecules include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, an isolated epitope binding fragment of an antibody, maxibodies, minibodies, nanobodies, intrabodies, diabodies, triabodies, tetrabodies, v-NAR and bis-scFv.

“Cardiovascular therapeutic” as used herein, refers to an exogenous polypeptide which modulates a cardiovascular function, e.g., reduces or alleviates a cause or symptom of a cardiovascular condition, e.g., a cardiac disorder or disease, or improves a value for a parameter associated with cardiovascular function, e.g., blood pressure. In embodiments, the cardiovascular therapeutic is a first or second exogenous polypeptide, which when present or expressed with the other exogenous polypeptide, modulates a cardiovascular function. In an embodiment, a first or second cardiovascular therapeutic has activity in the absence of the other.

As used herein, a “combination therapy” or “administered in combination” means that two (or more) different agents or treatments are administered to a subject as part of a treatment regimen for a particular disease or condition. The treatment regimen includes the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

The term “coordinated” or “coordinated manner” means that a plurality of agents work together to provide a therapeutic benefit. Types of coordinated activity include agent-additive, agent-synergistic, multiplicative, independent function, localization-based, proximity-dependent, scaffold-based, multimer-based, pathway-based, and compensatory activity. In an embodiment the level of therapeutic benefit conferred by a plurality of exogenous polypeptides delivered in the same enucleated erythroid cell is greater than would be seen if each of the plurality of polypeptides were delivered from different enucleated erythroid cells.

As used herein, “enucleated” refers to a cell, e.g., a reticulocyte or mature red blood cell that lacks a nucleus. In an embodiment an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In an embodiment an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell.

“Erythroid cell” as used herein, includes a nucleated red blood cell, a red blood cell precursor, an enucleated mature red blood cell, and a reticulocyte. For example, any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid cells are immortal or immortalized cells. For example, immortalized erythroblast cells can be generated by retroviral transduction of CD34+ hematopoietic progenitor cells to express Oct4, Sox2, Klf4, cMyc, and suppress TP53 (e.g., as described in Huang et al., Mol Ther. 22(2): 451-463, 2014). In addition, the cells may be intended for autologous use or provide a source for allogeneic transfusion. In some embodiments, erythroid cells are cultured. In an embodiment an erythroid cell is an enucleated red blood cell.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide is a polypeptide encoded by a nucleic acid that was introduced into the cell, which nucleic acid is optionally not retained by the cell. In some embodiments, an exogenous polypeptide is a polypeptide conjugated to the surface of the cell by chemical or enzymatic means.

“Metabolic therapeutic” as used herein, refers to an exogenous polypeptide which modulates a metabolic function, e.g., reduces or alleviates a cause or symptom of a metabolic disorder, e.g., a metabolic deficiency, or improves a value for a parameter associated with metabolic function, e.g., enzyme activity or levels of metabolites in a patient. In embodiments, the metabolic therapeutic is a first or second exogenous polypeptide, which when present or expressed with the other exogenous polypeptide, modulates a metabolic function. In an embodiment, a first or second metabolic therapeutic has activity in the absence of the other.

As used herein, the term “multimodal therapy” refers to a therapy, e.g., an enucleated erythroid cell therapy, that provides a plurality (e.g., 2, 3, 4, or 5 or more) of exogenous agents (e.g., polypeptides) that have a coordinated function (e.g., agent-additive, agent-synergistic, multiplicative, independent function, localization-based, proximity-dependent, scaffold-based, multimer-based, pathway-based, or compensatory activity).

As used herein, the term “pathway” or “biological pathway” refers to a plurality of biological molecules, e.g., polypeptides, which act together in a sequential manner. Examples of pathways include signal transduction cascades and complement cascades. In some embodiments, a pathway begins with detection of an extracellular signal and ends with a change in transcription of a target gene. In some embodiments, a pathway begins with detection of a cytoplasmic signal and ends with a change in transcription of a target gene. A pathway can be linear or branched. If branched, it can have a plurality of inputs (converging), or a plurality of outputs (diverging).

As used herein, a “proximity-dependent” molecule refers to a first molecule that has a different, e.g., greater, activity when in proximity with a second molecule than when alone. In some embodiments, a pair of proximity-dependent ligands activates a downstream factor more strongly when the ligands are in proximity than when they are distant from each other.

As used herein, “receptor component” refers to a polypeptide that functions as a receptor, by itself or as part of a complex. Thus, a receptor component encompasses a polypeptide receptor and a polypeptide that functions as part of a receptor complex.

The term “synergy” or “synergistic” means a more than additive effect of a combination of two or more agents (e.g., polypeptides that are part of an enucleated erythroid cell) compared to their individual effects. In certain embodiments, synergistic activity is a more-than-additive effect of an enucleated erythroid cell comprising a first polypeptide and a second polypeptide, compared to the effect of an enucleated erythroid cell comprising the first polypeptide and an enucleated erythroid cell comprising the second polypeptide. In some embodiments, synergistic activity is present when a first agent produces a detectable level of an output X, a second agent produces a detectable level of the output X, and the first and second agents together produce a more-than-additive level of the output X.

As used herein, the term “variant” of a polypeptide refers to a polypeptide having at least one sequence difference compared to that polypeptide, e.g., one or more substitutions, insertions, or deletions. In some embodiments, the variant has at least 70%, 80%, 85%, 90%, 95%, 96%, 97%, 98%, or 99% identity to that polypeptide. A variant includes a fragment. In some embodiments, a fragment lacks up to 1, 2, 3, 4, 5, 10, 20, or 100 amino acids on the N-terminus, C-terminus, or both (each independently), compared to the full-length polypeptide.

### Exemplary Exogenous Polypeptides and Uses Thereof

In embodiments, the erythroid cell therapeutics described herein comprise one or more (e.g., 2, 3, 4, 5, 6, 10 or more) different exogenous agents, e.g., exogenous polypeptides, lipids, or small molecules. In some embodiments, an enucleated erythroid cell therapeutic comprises an exogenous fusion polypeptide comprising two or more different proteins described herein. In some embodiments, an enucleated erythroid cell comprises two or more different exogenous polypeptides described herein. In some embodiments, one or more (e.g., all) of the exogenous polypeptides are human polypeptides or fragments or variants thereof.

In some embodiments, the two or more polypeptides act on the same target, and in other embodiments, they act on two or more different targets. In some embodiments, the single target or plurality of targets is chosen from an endogenous human protein or a soluble factor (e.g., a polypeptide, small molecule, or cell-free nucleic acid).

One or more of the exogenous proteins may have post-translational modifications characteristic of eukaryotic cells, e.g., mammalian cells, e.g., human cells. In some embodiments, one or more (e.g., 2, 3, 4, 5, or more) of the exogenous proteins are glycosylated, phosphorylated, or both. In vitro detection of glycoproteins can be accomplished on SDS-PAGE gels and Western Blots using a modification of Periodic acid-Schiff (PAS) methods. Cellular localization of glycoproteins can be accomplished utilizing lectin fluorescent conjugates known in the art. Phosphorylation may be assessed by Western blot using phospho-specific antibodies. Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

In some embodiments, one or more of the exogenous polypeptides is a fusion protein, e.g., is a fusion with an endogenous red blood cell protein or fragment thereof, e.g., a transmembrane protein, e.g., GPA or a transmembrane fragment thereof. In some embodiments, one or more of the exogenous polypeptides is fused with a domain that promotes dimerization or multimerization, e.g., with a second fusion exogenous polypeptide, which optionally comprises a dimerization domain. In some embodiments, the dimerization domain comprises a portion of an antibody molecule, e.g., an Fc domain or CH3 domain. In some embodiments, the first and second dimerization domains comprise knob-in-hole mutations (e.g., a T366Y knob and a Y407T hole) to promote heterodimerization.

An exemplary human polypeptide, e.g., a human polypeptide selected from any of Tables 1-4, includes:

a) a naturally occurring form of the human polypeptide, e.g., a naturally occurring form of the human polypeptide that is not associated with a disease state;

b) the human polypeptide having a sequence appearing in a database, e.g., GenBank database, on Jul. 19, 2017, for example a naturally occurring form of the human polypeptide that is not associated with a disease state having a sequence appearing in a database, e.g., GenBank database, on Jul. 19, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity) from a human polypeptide having the sequence of a) or b) . Candidate peptides under f) can be made and screened for similar activity as described herein and would be equivalent hereunder if expressed in enucleated erythroid cells as described herein).

In embodiments, an exogenous polypeptide comprises a human polypeptide or fragment thereof, e.g., all or a fragment of a human polypeptide of a), b), c), d), e), or f) of the preceding paragraph. In an embodiment, the exogenous polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human polypeptide.

The invention contemplates that functional fragments or variants thereof (e.g., a ligand-binding fragment or variant thereof or enzymatically active fragment or variant thereof, e.g., of the proteins listed in Table 1, 2, 3 or 4) can be made and screened for similar activity as described herein and would be equivalent hereunder if expressed in enucleated erythroid cells as described herein).

In embodiments, the two or more exogenous agents (e.g., polypeptides) have related functions that are agent-additive, agent-synergistic, multiplicative, independent function, localization-based, proximity-dependent, scaffold-based, multimer-based, pathway-based, or compensatory, as described herein. In some embodiments, more than one of these descriptors applies to a given erythroid cell.

Particular exogenous polypeptides that can be present or expressed in an erythroid cell are now described in greater detail.

### Exogenous GLP-1 Polypeptides

In some embodiments, the exogenous polypeptide comprises GLP-1 or a fragment or variant thereof. For example, an exogenous GLP-1 polypeptide can comprise a sequence of SEQ ID NO: 1, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the GLP-1 polypeptide has at least one activity characteristic of a GLP-1 polypeptide of SEQ ID NO: 1, e.g., it can bind a GLP-1 receptor, e.g., with a Kd no greater than 10%, 20%, 50%, 2-fold, or 5-fold the Kd of a GLP-1 polypeptide of SEQ ID NO: 1 for the GLP-1 receptor. Receptor binding can be measured, e.g., according to Donnelly, “The structure and function of the glucagon-like peptide-1 receptor and its ligands.” Br J Pharmacol. 2012 May; 166(1):27-41, which is herein incorporated by reference in its entirety. In embodiments, the GLP-1 polypeptide has one or more of the following characteristics: potentiates the glucose-induced secretion of insulin from pancreatic beta cells, increases insulin expression, inhibits beta-cell apoptosis, promotes beta-cell neogenesis, increases insulin sensitivity e.g., in alpha and/or beta cells, increases beta cell mass and insulin expression, reduces glucagon secretion, delays gastric emptying, promotes satiety and increases peripheral glucose disposal. Functional GLP-1 polypeptides are described, e.g., in Donnelly, (supra). In embodiments, the GLP-1 polypeptide comprises GLP-1(1-37), GLP-1(7-37), or GLP-1(7-36)amide. In embodiments, the GLP-1 polypeptide is amidated. In embodiments, the GLP-1 polypeptide has a half-life of greater than 2, 3, 5, or 10 minutes in a subject, e.g., a human subject.

In some embodiments, an erythroid cell described herein is contacted with, or comprises, a nucleic acid sequence (e.g., DNA or RNA) comprising a GLP-1 polypeptide described herein.

In some embodiments, an erythroid cell described herein comprises a GLP-1 polypeptide as described herein and an insulin polypeptide, e.g., as described herein.

In some embodiments, the GLP-1 polypeptide comprises a GLP-1 extracellular domain (or fragment or variant thereof) and a membrane-anchor or transmembrane domain, e.g., a heterologous transmembrane domain, e.g., GPA.

### Insulin Polypeptides

In some embodiments, the exogenous polypeptide comprises insulin or a fragment or variant thereof. For example, an exogenous insulin polypeptide can comprise a sequence of SEQ ID NO: 2, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the insulin polypeptide has at least one activity characteristic of an insulin polypeptide of SEQ ID NO: 2, e.g., it can bind an insulin receptor, e.g., with Kd no greater than 10%, 20%, 50%, 2-fold, or 5-fold the Kd of an insulin polypeptide of SEQ ID NO: 2 for the insulin receptor. Binding may be measured, e.g., as described in Kristensen et al., “Alanine scanning mutagenesis of insulin” J Biol Chem. 1997 May 16; 272(20):12978-83, which is herein incorporated by reference in its entirety. In embodiments, the insulin polypeptide has one or more of the following characteristics: binds the insulin receptor or promotes glucose uptake, e.g., by adipose, hepatic, or skeletal muscle cells. Functional insulin polypeptides are described, e.g., in Kristensen et al., (supra). In embodiments, the insulin polypeptide comprises insulin lispro, insulin aspart, insulin glulisine, insulin detemir, insulin degludec, or insulin glargine.

In some embodiments, an erythroid cell described herein is contacted with, or comprises, a nucleic acid sequence (e.g., DNA or RNA) comprising an insulin polypeptide described herein.

In some embodiments, an erythroid cell described herein comprises an insulin polypeptide as described herein and a GLP-1 polypeptide, e.g., as described herein.

### FGF-21 Polypeptides

In some embodiments, the exogenous polypeptide comprises FGF-21 or a fragment or variant thereof. For example, an exogenous FGF-21 polypeptide can comprise a sequence of SEQ ID NO: 5, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the FGF-21 polypeptide has at least one activity characteristic of an FGF-21 polypeptide of SEQ ID NO: 5, e.g., it can bind an FGF-21 receptor, e.g., with Kd no greater than 10%, 20%, 50%, 2-fold, or 5-fold the Kd of an

FGF-21 polypeptide of SEQ ID NO: 5 for the FGF-21 receptor. In embodiments, the FGF-21 polypeptide has one or more of the following characteristics: binds an FGF-21 receptor or promotes glucose uptake, e.g., by adipose cells. Functional FGF-21 polypeptides are described, e.g., in Adams A C et al. “LY2405319, an Engineered FGF-21 Variant, Improves the Metabolic Status of Diabetic Monkeys.” PLoS One. 2013 Jun. 18; 8(6):e65763 and Lee J H et al., “An engineered FGF21 variant, LY2405319, can prevent non-alcoholic steatohepatitis by enhancing hepatic mitochondrial function.” Am J Transl Res. 2016 Nov 15;8(11):4750-4763, each of which is herein incorporated by reference in its entirety.

In some embodiments, an erythroid cell described herein is contacted with, or comprises, a nucleic acid sequence (e.g., DNA or RNA) comprising an FGF-21 polypeptide described herein.

In some embodiments, an erythroid cell described herein comprises an FGF-21 polypeptide as described herein and a glucagon polypeptide, e.g., as described herein.

In some embodiments, the FGF-21 polypeptide comprises a FGF-21 extracellular domain (or fragment or variant thereof) and a membrane-anchor or transmembrane domain, e.g., a heterologous transmembrane domain, e.g., GPA.

### Leptin Polypeptides

In some embodiments, the exogenous polypeptide comprises leptin or a fragment or variant thereof. For example, an exogenous leptin polypeptide can comprise a sequence of SEQ ID NO: 7, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the leptin polypeptide has at least one activity characteristic of a leptin polypeptide of SEQ ID NO: 7, e.g., it can bind a leptin receptor, e.g., with a Kd no greater than 10%, 20%, 50%, 2-fold, or 5-fold the Kd of a leptin polypeptide of SEQ ID NO: 7 for the leptin receptor. In embodiments, the leptin polypeptide has one or more of the following characteristics: binds a leptin receptor or decreases hunger. Functional leptin polypeptides are described, e.g., in Khaled, Tomini. The isolation of novel leptin variants using phage display. PhD thesis. The University of Sheffield, 2015, which is herein incorporated by reference in its entirety.

In some embodiments, an erythroid cell described herein is contacted with, or comprises, a nucleic acid sequence (e.g., DNA or RNA) comprising a leptin polypeptide described herein.

In some embodiments, an erythroid cell described herein comprises a leptin polypeptide as described herein and a glucagon polypeptide, e.g., as described herein.

### Glucagon Polypeptides

In some embodiments, the exogenous polypeptide comprises glucagon or a fragment or variant thereof. For example, an exogenous glucagon polypeptide can comprise a sequence of SEQ ID NO: 6, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the glucagon polypeptide has at least one activity characteristic of a glucagon polypeptide of SEQ ID NO: 6, e.g., it can bind a glucagon receptor, e.g., with a Kd no greater than 10%, 20%, 50%, 2-fold, or 5-fold the Kd of a glucagon polypeptide of SEQ ID NO: 6 for the glucagon receptor. In embodiments, the glucagon polypeptide has one or more of the following characteristics: binds a glucagon receptor or promoting gluconeogenesis and/or glycogenolysis, e.g., in hepatic cells. Functional glucagon polypeptides are described, e.g., in Chabenne et al., “A glucagon analog chemically stabilized for immediate treatment of life-threatening hypoglycemia.” Mol Metab. 2014 Jan. 22; 3(3):293-300, which is herein incorporated by reference in its entirety.

In some embodiments, an erythroid cell described herein is contacted with, or comprises, a nucleic acid sequence (e.g., DNA or RNA) comprising a glucagon polypeptide described herein.

In some embodiments, an erythroid cell described herein comprises a glucagon polypeptide as described herein and an FGF-21 polypeptide and/or a leptin polypeptide, e.g., as described herein.

Tissue Plasminogen Activator (tPA) Polypeptides

In some embodiments, the exogenous polypeptide comprises tPA or a fragment or variant thereof. For example, an exogenous tPA polypeptide can comprise a sequence of any of SEQ ID NOs: 10-13, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the tPA polypeptide has at least one activity characteristic of a tPA polypeptide of any of SEQ ID NOs: 10-13, e.g., it can convert plasminogen to plasmin, e.g., with an reaction rate constant at least 90%, 80%, 70%, 60%, or 50% of that of a tPA polypeptide of any of SEQ ID NOs: 10-13. Functional tPA polypeptides are described, e.g., in Benedict, et al. “New Variant of Human Tissue Plasminogen Activator (TPA) With Enhanced Efficacy and Lower Incidence of Bleeding Compared with Recombinant Human TPA.” Circulation. 1995;92:3032-3040 or “Ahern, et al. Site-directed Mutagenesis in Human Tissue-Plasminogen Activator. JBC 265(10) 5540-5545. 1990”, each of which is herein incorporated by reference in its entirety. Exemplary tPA polypeptides include alteplase, reteplase, tenecteplase, and desmoteplase.

In some embodiments, an erythroid cell described herein is contacted with, or comprises, a nucleic acid sequence (e.g., DNA or RNA) comprising a tPA polypeptide described herein.

In some embodiments, an erythroid cell described herein comprises a tPA polypeptide as described herein and a TFPI polypeptide, e.g., as described herein.

### Tissue Factor Pathway Inhibitor (TFPI) Polypeptides

In some embodiments, the exogenous polypeptide comprises TFPI or a fragment or variant thereof. For example, an exogenous TFPI polypeptide can comprise a sequence of SEQ ID NO: 14, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the TFPI polypeptide has at least one activity characteristic of a TFPI polypeptide of SEQ ID NO: 14, e.g., it can inhibit Factor Xa, e.g., at least 90%, 80%, 70%, 60%, or 50% as strongly as does a polypeptide of SEQ ID NO: 14. Functional TFPI polypeptides are described, e.g., in Johnson K et al. “Activity of secreted Kunitz domain 1 variants of tissue factor pathway inhibitor.” Thromb Haemost. 1998 October; 80(4):585-7, Petersen JG et al. “Characterization of human tissue factor pathway inhibitor variants expressed in Saccharomyces cerevisiae.” J Biol Chem. 1993 Jun. 25; 268(18):13344-51, Maroney et al. “New insights into the biology of tissue factor pathway inhibitor.” Journal of thrombosis and haemostasis. 2015; 13:S200-S207, each of which is herein incorporated by reference in its entirety.

In some embodiments, an erythroid cell described herein is contacted with, or comprises, a nucleic acid sequence (e.g., DNA or RNA) comprising a TFPI polypeptide described herein.

In some embodiments, an erythroid cell described herein comprises a TFPI polypeptide as described herein and a tPA polypeptide, e.g., as described herein.

### Brain Natriuretic Peptide (BNP) Polypeptides

In some embodiments, the exogenous polypeptide comprises BNP or a fragment or variant thereof. For example, an exogenous BNP polypeptide can comprise a sequence of SEQ ID NO: 8, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the BNP polypeptide has at least one activity characteristic of a BNP polypeptide of SEQ ID NO: 8, e.g., it can bind NPRA, e.g., with a Kd no greater than 10%, 20%, 50%, 2-fold, or 5-fold the Kd a BNP polypeptide of SEQ ID NO: 8 for NPRA. In embodiments, the BNP polypeptide binds to and activates the atrial natriuretic factor receptor NPRA, and/or NPRB. Functional BNP polypeptides are described, e.g., in Schoenfeld et al. “Mutations in B-type natriuretic peptide mediating receptor-A selectivity” FEBS Lett. 1997 Sep. 8;414(2):263-7, which is herein incorporated by reference in its entirety.

In some embodiments, an erythroid cell described herein is contacted with, or comprises, a nucleic acid sequence (e.g., DNA or RNA) comprising a BNP polypeptide described herein.

In some embodiments, an erythroid cell described herein comprises a BNP polypeptide as described herein and a relaxin polypeptide, e.g., as described herein.

### Relaxin Polypeptides

In some embodiments, the exogenous polypeptide comprises relaxin or a fragment or variant thereof. For example, an exogenous relaxin polypeptide (e.g., relaxin 2) can comprise a sequence of SEQ ID NO: 9 and SEQ ID NO: 31, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the relaxin polypeptide has at least one activity characteristic of a relaxin polypeptide of SEQ ID NO: 9 and SEQ ID NO: 31, e.g., it can bind H2 relaxin receptor, RXFP1, e.g., with a Kd no greater than 10%, 20%, 50%, 2-fold, or 5-fold the Kd of a relaxin polypeptide of SEQ ID NO: 9 and SEQ ID NO: 31 for RXFP1. Binding can be measured, e.g., by an assay of Hossain et al. “The Minimal Active Structure of Human Relaxin-2” The Journal of Biological Chemistry Vol. 286, NO. 43, pp. 37555-37565, Oct. 28, 2011, which is herein incorporated by reference in its entirety. In embodiments, the relaxin polypeptide antagonizes endothelin-1 and/or angiotensin II, or binds to and activate RXFP2. Functional relaxin polypeptides are described, e.g., in Hossain et al. (supra). In embodiments the relaxin peptide comprises an A chain (e.g., of SEQ ID NO: 9 or a fragment or variant thereof) and a B chain (e.g., of SEQ ID NO: 34 or a fragment or variant thereof). In embodiments, the A and B chain are linked, e.g., by a polypeptide linker or one or more (e.g., 2 or 3) disulfide bonds.

In some embodiments, an erythroid cell described herein is contacted with, or comprises, a nucleic acid sequence (e.g., DNA or RNA) comprising a relaxin polypeptide described herein.

In some embodiments, an erythroid cell described herein comprises a relaxin polypeptide (e.g., relaxin 2) as described herein and a BNP polypeptide and/or a myeloperoxidase polypeptide, e.g., as described herein.

### Anti-RANK-L Polypeptides, e.g., Antibody Molecules

In some embodiments, the exogenous polypeptide comprises an anti-RANK-L antibody molecule or a fragment or variant thereof, e.g., an scFv. For example, an exogenous anti-RANK-L antibody molecule can comprise a heavy chain sequence of SEQ ID NO: 15 and/or a light chain sequence of SEQ ID NO: 16, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the anti-RANK-L antibody molecule (e.g., scFv) comprises a HC CDR1, HC CDR2, and HC CDR3 of SEQ ID NO: 15 and a LC CDR1, LC CDR2, and LC CDR3 of SEQ ID NO: 16, e.g., according to Kabat or Chothia. In embodiments, the anti-RANK-L antibody molecule (e.g., scFv) comprises a heavy chain variable region of SEQ ID NO: 15 and a light chain variable region of SEQ ID NO: 16. In embodiments, the anti-RANK-L antibody comprises denosumab scFv. In embodiments, the anti-RANK-L antibody molecule has at least one activity characteristic of an anti-RANK-L antibody molecule of SEQ ID NO: 15 and 16, e.g., it can bind RANK-L, e.g., with a Kd no greater than 10%, 20%, 50%, 2-fold, or 5-fold the Kd of an anti-RANK-L antibody molecule of SEQ ID NO: 15 and 16 for RANK-L.

In some embodiments, an erythroid cell described herein is contacted with, or comprises, a nucleic acid sequence (e.g., DNA or RNA) comprising an anti-RANK-L antibody molecule described herein.

In some embodiments, an erythroid cell described herein comprises an anti-RANK-L antibody molecule as described herein and a PTH polypeptide, e.g., as described herein.

### Parathyroid Hormone (PTH) Polypeptides

In some embodiments, the exogenous polypeptide comprises PTH or a fragment or variant thereof. For example, an exogenous PTH polypeptide can comprise a sequence of SEQ ID NO: 17, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the PTH polypeptide has at least one activity characteristic of a PTH polypeptide of SEQ ID NO: 17, e.g., it can bind parathyroid hormone 1 receptor, e.g., with a Kd no greater than 10%, 20%, 50%, 2-fold, or 5-fold the Kd of a PTH polypeptide of SEQ ID NO: 17 for parathyroid hormone 1 receptor. Functional PTH polypeptides are described, e.g., in Gardella et al., “Analysis of parathyroid hormone's principal receptor-binding region by site-directed mutagenesis and analog design.” Endocrinology. 1993 May;132(5):2024-30 and Shimizu et al., “Minimization of parathyroid hormone, novel amino-terminal parathyroid hormone fragments with enhanced potency in activating the type-1 parathyroid hormone receptor” the Journal of Biological Chemistry Vol. 275, No. 29, Issue of July 21, pp. 21836-21843, 2000.

In some embodiments, an erythroid cell described herein is contacted with, or comprises, a nucleic acid sequence (e.g., DNA or RNA) comprising a PTH polypeptide described herein. In some embodiments, an erythroid cell described herein comprises a PTH polypeptide as described herein and an anti-RANK-L antibody molecule, e.g., as described herein.

### Phenylalanine Ammonia Lyase (PAL) Polypeptides

In some embodiments, the exogenous polypeptide comprises PAL or a fragment or variant thereof. For example, an exogenous PAL polypeptide can comprise a sequence of SEQ ID NO: 18, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the PAL polypeptide has at least one activity characteristic of a PAL polypeptide of SEQ ID NO: 18, e.g., it can metabolize phenylalanine, e.g., with reaction rate constant at least 90%, 80%, 70%, 60%, or 50% of that of a PAL polypeptide of SEQ ID NO: 18. Phenylalanine metabolism can be measured, e.g., using an assay of Want et al. Structural and biochemical characterization of the therapeutic Anabaena variabilis phenylalanine ammonia lyase. J Mol Biol. 2008 July; 380(4): 623-635, which is herein incorporated by reference in its entirety. Functional PAL polypeptides are described, e.g., in Want et al., (supra), Moffitt et al. “Discovery of Two Cyanobacterial Phenylalanine Ammonia Lyases: Kinetic and Structural Characterization.” Biochem. 2007 January; 46(4): 1004-1012, Jaliani et al. “Engineering and kinetic stabilization of the therapeutic enzyme Anabeana variabilis phenylalanine ammonia lyase.” Appl Biochem Biotechnol. 2013 December; 171(7): 1805-1818, and Jaliani et al. “Engineering and stabilization of A. variabilis phenylalanine ammonia lyase. Molecular Biology Research Communications.” 2014. 3: 202, each of which is herein incorporated by reference in its entirety. In embodiments, the PAL polypeptide comprises cyanobacterial PAL, e.g., Anabaena variabilis PAL, or a fragment or variant thereof.

In some embodiments, an erythroid cell described herein is contacted with, or comprises, a nucleic acid sequence (e.g., DNA or RNA) comprising a PAL polypeptide described herein.

In some embodiments, an erythroid cell described herein comprises a PAL polypeptide as described herein and a phenylalanine transporter polypeptide (e.g., a TAT1 polypeptide), e.g., as described herein.

### T-Type Amino-Acid Transporter-1 (TAT1) Polypeptides

In some embodiments, the exogenous polypeptide comprises TAT1 or a fragment or variant thereof. For example, an exogenous TAT1 polypeptide can comprise a sequence of SEQ ID NO: 19, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the TAT1 polypeptide has at least one activity characteristic of a TAT1 polypeptide of SEQ ID NO: 19, e.g., it can transport phenylalanine, e.g., at a rate at least 90%, 80%, 70%, 60%, or 50% of that of a TAT1 polypeptide of SEQ ID NO: 19. Functional TAT1 polypeptides are described, e.g., in Medici et al. “A large-scale association analysis of 68 thyroid hormone pathway genes with serum TSH FT4 levels. Eur J Endocrinol. 2011 May; 164(5): 781-788” which is herein incorporated by reference in its entirety.

In some embodiments, an erythroid cell described herein is contacted with, or comprises, a nucleic acid sequence (e.g., DNA or RNA) comprising a TAT1 polypeptide described herein.

In some embodiments, an erythroid cell described herein comprises a TAT1 polypeptide as described herein and a PAL polypeptide, e.g., as described herein.

In some embodiments, the TAT1 polypeptide comprises a TAT1 extracellular domain (or fragment or variant thereof) and a membrane-anchor or transmembrane domain, e.g., a heterologous transmembrane domain, e.g., GPA.

### Cystathionine B-Synthase (CBS) Polypeptides

In some embodiments, the exogenous polypeptide comprises CBS or a fragment or variant thereof. For example, an exogenous CBS polypeptide can comprise a sequence of SEQ ID NO: 20, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the CBS polypeptide has at least one activity characteristic of a CBS polypeptide of SEQ ID NO: 20, e.g., it can convert homocysteine into cystathionine, e.g., with reaction rate constant at least 90%, 80%, 70%, 60%, or 50% of that of a CBS polypeptide of SEQ ID NO: 20. Reaction rate can be assayed, e.g., according to Kozich et al. Cystathionine beta-synthase mutations: effect of mutation topology on folding and activity. Hum Mutat. 2010 July; 31(7): 809-819, which is herein incorporated by reference in its entirety. Functional CBS polypeptides are described, e.g., in Kozich et al. (supra), Bublil et al. J “Enzyme replacement with PEGylated cystathionine B-synthase ameliorates homocystinuria in murine model” Clin Invest. 2016 June; 126(6): 2372-2384, Singh et al. “Cystathionine B-synthase deficiency: Effects of betaine supplementation after methionine restriction in B6-nonresponsive homocystinuria.” Genetics in Medicine. 2004; 6:90-95, Ereno-Orbea et al. “Structural insight into the molecular mechanism of allosteric activation of human cystathionine B-synthase by S-adenosylmethionine.” Proc Natl Acad Sci; 2014 September; 111(37), Lee et al. “Identification and functional analysis of cystathionine beta-synthase gene mutations in patients with homocystinuria.” Journal of Human Genetics. 2005 December; 50(12): 648-654, Fowler et al. “Homocystinuria: Evidence for three distinct classes of cytathionine b-synthase mutants in cultured fibroblasts.” J Clin Invest. 1978; 61: 645-653, Shan et al. “Mutations in the regulatory domain of cystathionine B-synthase can functionally suppress patient-derived mutations in cis.” Hum Mol Genet. 2001; 10(6): 635-643, Dimster-Denk et al. “Mono and Dual Cofactor Dependence of Human Cystathionine B-synthase Enzyme Variants In Vivo and In Vitro.” Genes, Genomes, Genetics. 2013; 3(10): 1619-1628, Casique et al. “Characterization of two pathogenic mutations in cystathionine beta-synthase.” Gene. 2013; 531(1), Sirachainan et al. “A Novel Mutation of Cytathionine B-synthase Gene in a Thai Boy With Homocystinuria.” J Pediatr Hematol Oncol; 31(10): 768, and Janosik et al. “Impaired Heme Binding and Aggregation of Mutant Cystathionin B-Synthase Subunits in Homocystinuria.” Am J Hum Genet. 2001; 68: 1506-1513, each of which is herein incorporated by reference in its entirety.

In some embodiments, an erythroid cell described herein is contacted with, or comprises, a nucleic acid sequence (e.g., DNA or RNA) comprising a CBS polypeptide described herein.

In some embodiments, an erythroid cell described herein comprises a CBS polypeptide as described herein and a transporter polypeptide, e.g., a L-homocysteine or L-serine transporter polypeptide, e.g., as described herein.

In some embodiments, the CBS polypeptide comprises a CBS extracellular domain (or fragment or variant thereof) and a membrane-anchor or transmembrane domain, e.g., a heterologous transmembrane domain, e.g., GPA.

### Sodium-Coupled Neutral Amino Acid Transporter 2 (SAT2) Polypeptides

In some embodiments, the exogenous polypeptide comprises SAT2 or a fragment or variant thereof. For example, an exogenous SAT2 polypeptide can comprise a sequence of SEQ ID NO: 21, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the SAT2 polypeptide has at least one activity characteristic of a SAT2 polypeptide of SEQ ID NO: 21, e.g., it can transport alpha-(methylamino)isobutyric acid, e.g., at a rate at least 90%, 80%, 70%, 60%, or 50% of that of a SAT2 polypeptide of SEQ ID NO: 21. Transport can be measured, e.g., using an assay of Hatanaka et al. “Primary structure, functional characteristics and tissue expression pattern of human ATA2, a subtype of amino acid transport system A.” Biochim Biophys Acta. 2000 Jul. 31; 1467(1):1-6., which is herein incorporated by reference in its entirety.

In some embodiments, an erythroid cell described herein is contacted with, or comprises, a nucleic acid sequence (e.g., DNA or RNA) comprising a SAT2 polypeptide described herein.

In some embodiments, an erythroid cell described herein comprises a SAT2 polypeptide as described herein and a CBS polypeptide, e.g., as described herein.

In some embodiments, the SAT2 polypeptide comprises a SAT2 extracellular domain (or fragment or variant thereof) and a membrane-anchor or transmembrane domain, e.g., a heterologous transmembrane domain, e.g., GPA.

### Neutral Amino Acid Transporter A (ASCT1) Polypeptides

In some embodiments, the exogenous polypeptide comprises ASCT1 or a fragment or variant thereof. For example, an exogenous ASCT1 polypeptide can comprise a sequence of SEQ ID NO: 22, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the ASCT1 polypeptide has at least one activity characteristic of an ASCT1 polypeptide of SEQ ID NO: 22, e.g., it can transport serine, e.g., at a rate at least 90%, 80%, 70%, 60%, or 50% of that of an ASCT1 polypeptide of SEQ ID NO: 22. Transport can be measured, e.g., using an assay of Scopelliti et al. “Na+ interactions with the neutral amino acid transporter ASCT1.” J Biol Chem. 2014; 289(25): 17468-17479, which is herein incorporated by reference in its entirety. Functional ASCT1 polypeptides are described, e.g., in Scopelliti et al., (supra) and Bennett et al. Mutation screening of a neutral amino acid transporter, ASCT1, and its potential role in schizophrenia. Psychiatr Genet. 2000; 10(2): 79-82, each of which is herein incorporated by reference in its entirety.

In some embodiments, an erythroid cell described herein is contacted with, or comprises, a nucleic acid sequence (e.g., DNA or RNA) comprising an ASCT1 polypeptide described herein.

In some embodiments, an erythroid cell described herein comprises an ASCT1 polypeptide as described herein and a CBS polypeptide, e.g., as described herein.

In some embodiments, the ASCT1 polypeptide comprises an ASCT1 extracellular domain (or fragment or variant thereof) and a membrane-anchor or transmembrane domain, e.g., a heterologous transmembrane domain, e.g., GPA.

### Uricase Polypeptides

In some embodiments, the exogenous polypeptide comprises uricase or a fragment or variant thereof. For example, an exogenous uricase polypeptide can comprise a sequence of SEQ ID NO: 23, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the uricase polypeptide has at least one activity characteristic of a uricase polypeptide of SEQ ID NO: 18, e.g., it can convert uric acid to 5-hydroxyisourate, e.g., with reaction rate constant at least 90%, 80%, 70%, 60%, or 50% of that of a uricase polypeptide of SEQ ID NO: 23. Activity can be measured, e.g., by an assay according to Zhang et al. “Construction, expression, purification and characterization of mutant of Aspergillus flavus urate oxidase.” Sheng Wu Gong Cheng Xue Bao. 2010; 26(8): 1102-1107, which is herein incorporated by reference in its entirety. Functional uricase proteins are described, e.g., in Zhang et al. (supra). In embodiments, the uricase polypeptide comprises Aspergillus flavus urate oxidase, or a fragment or variant thereof.

In some embodiments, an erythroid cell described herein is contacted with, or comprises, a nucleic acid sequence (e.g., DNA or RNA) comprising a uricase polypeptide described herein.

In some embodiments, an erythroid cell described herein comprises a uricase polypeptide as described herein and a catalase polypeptide, e.g., as described herein.

### Catalase Polypeptides

In some embodiments, the exogenous polypeptide comprises catalase or a fragment or variant thereof. For example, an exogenous catalase polypeptide can comprise a sequence of SEQ ID NO: 24, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the catalase polypeptide has at least one activity characteristic of a catalase polypeptide of SEQ ID NO: 24, e.g., it can catalyze decomposition of hydrogen peroxide to water and oxygen (catalatic activity), e.g., with reaction rate constant at least 90%, 80%, 70%, 60%, or 50% of that of a catalase polypeptide of SEQ ID NO: 24. Catalatic activity can be measured, e.g., using an assay of Kirkman et al., “The function of catalase-bound NADPH.” J Biol Chem. 1987 Jan. 15;262(2):660-6, which is herein incorporated by reference in its entirety. Functional catalase polypeptides are described, e.g., in ⋅

Goth et al. Catalase enzyme mutations and their association with diseases. Mol Diagn. 2004; 8(3): 141-149, Otera et al. Pex5p Imports Folded Tetrameric Catalase by Interaction with Pex13p. Traffic. 2012; 13: 1364-1377, Goth et al. A novel catalase mutation detected by polymerase chain reaction-single strand conformation polymorphism, nucleotide sequencing, and Western blot analyses is responsible for the type C of Hungarian acatalasemia. Electrophoresis. 2001; 22(1)., and Kodydkova et al. Human Catalase, Its Polymorphisms, Regulation and Changes of Its Activity in Different Diseases. Folia Biologica. 2014; 60: 153-167, each of which is herein incorporated by reference in its entirety.

In some embodiments, an erythroid cell described herein is contacted with, or comprises, a nucleic acid sequence (e.g., DNA or RNA) comprising a catalase polypeptide described herein.

In some embodiments, an erythroid cell described herein comprises a catalase polypeptide as described herein and an uricase polypeptide, e.g., as described herein.

### Coagulation Factor X or Xa Polypeptides

In some embodiments, the exogenous polypeptide comprises Factor X or Xa or a fragment or variant thereof. For example, an exogenous Factor X or Xa polypeptide can comprise a sequence of SEQ ID NO: 25, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the

Factor X or Xa polypeptide has at least one activity characteristic of a Factor X polypeptide of SEQ ID NO: 25, e.g., it can cleave prothrombin to thrombin (e.g., in the presence of Factor Va) e.g., with reaction rate constant at least 90%, 80%, 70%, 60%, or 50% of that of a Factor X polypeptide of SEQ ID NO: 25.

In some embodiments, an erythroid cell described herein is contacted with, or comprises, a nucleic acid sequence (e.g., DNA or RNA) comprising a Factor X polypeptide described herein.

### Coagulation Factor VII or VIIa Polypeptides

In some embodiments, the exogenous polypeptide comprises Factor VII or VIIa or a fragment or variant thereof. For example, an exogenous Factor VII or VIIa polypeptide can comprise a sequence of SEQ ID NO: 26, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the Factor VII or VIIa polypeptide has at least one activity characteristic of a Factor VII polypeptide of SEQ ID NO: 26, e.g., it can cleave Factor X to Factor Xa e.g., with reaction rate constant at least 90%, 80%, 70%, 60%, or 50% of that of a Factor X polypeptide of SEQ ID NO: 26.

In some embodiments, an erythroid cell described herein is contacted with, or comprises, a nucleic acid sequence (e.g., DNA or RNA) comprising a Factor VII or VIla polypeptide described herein.

### Anti-TFPI Polypeptides, e.g., Antibody Molecules

In some embodiments, the exogenous polypeptide comprises an anti-TFPI antibody molecule or a fragment or variant thereof, e.g., an scFv. For example, an exogenous anti-TFPI antibody molecule can comprise a sequence of SEQ ID NO: 27 or SEQ ID NO: 28, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the anti-TFPI antibody molecule (e.g., scFv) comprises a HC CDR1, HC CDR2, HC CDR3, LC CDR1, LC CDR2, and LC CDR3 of SEQ ID NO: 27 or 28, e.g., according to Kabat or Chothia. In embodiments, the anti-TFPI antibody molecule (e.g., scFv) comprises a heavy chain variable region and a light chain variable region of SEQ ID NO: 27 or 28. In embodiments, the anti-TFPI antibody molecule has at least one activity characteristic of an anti-TFPI antibody molecule of SEQ ID NO: 27 or 28, e.g., it can bind TFPI, e.g., with a Kd no greater than 10%, 20%, 50%, 2-fold, or 5-fold the Kd of an anti-TFPI antibody molecule of SEQ ID NO: 27 or 28 for TFPI.

In some embodiments, an erythroid cell described herein is contacted with, or comprises, a nucleic acid sequence (e.g., DNA or RNA) comprising an anti-TFPI antibody molecule described herein.

### Anti-FIXa/FX Polypeptides, e.g., Antibody Molecules

In some embodiments, the exogenous polypeptide comprises an anti-FIXa/FX antibody molecule or a fragment or variant thereof, e.g., an scFv. For example, an exogenous anti-FIXa/FX antibody molecule can comprise a sequence of SEQ ID NO: 29, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the anti-FIXa/FX antibody molecule (e.g., scFv) comprises a HC CDR1, HC CDR2, HC CDR3, LC CDR1, LC CDR2, and LC CDR3 of SEQ ID NO: 29, e.g., according to Kabat or Chothia. In embodiments, the anti-FIXa/FX antibody molecule (e.g., scFv) comprises a heavy chain variable region and a light chain variable region of SEQ ID NO: 29. In embodiments, the anti-FIXa/FX antibody molecule has at least one activity characteristic of an anti-FIXa/FX antibody molecule of SEQ ID NO: 29, e.g., it can bind FIXa/FX, e.g., with a Kd no greater than 10%, 20%, 50%, 2-fold, or 5-fold the Kd of an anti-FIXa/FX antibody molecule of SEQ ID NO: 29 for FIXa/FX.

In some embodiments, an erythroid cell described herein is contacted with, or comprises, a nucleic acid sequence (e.g., DNA or RNA) comprising an anti-FIXa/FX antibody molecule described herein.

### Agent-Additive Configurations

When two or more agents (e.g., polypeptides) are agent-additive, the effect of the agents acting together is greater than the effect of either agent acting alone. In an embodiment, two agents have different (e.g., complementary) functions in the erythroid cell (e.g., on the erythroid cell surface) and act together to have a stronger effect (compared to either of the agents acting alone), e.g., a higher binding affinity for the target, or a greater degree of modulation of signal transduction by the target, e.g., a single target. In some embodiments, two or more agents each bind to the same target, e.g., to different epitopes within the same target protein.

In an embodiment, the agents associate with one another, e.g., are members of a heterodimeric complex. In an embodiment, the agents have greater avidity for a target when acting together than when acting alone.

In some embodiments, the two or more agents enable tighter binding to a target than either agent alone. In some embodiments, a heterodimer of receptor components, e.g., cytokine receptor components, e.g., interleukin receptor components, e.g., IL-1 receptor components, bind to a target, e.g., IL-1, with higher affinity than either receptor component alone. Many signaling molecules form heterodimers or heteromultimers on the cell surface to bind to their ligand. Cytokine receptors, for example, can be heterodimers or heteromultimers. For instance, IL-2 receptor comprises three different molecules: IL2Ra, IL2Rb, and IL2Rg. The IL-13 receptor is a heterodimer of IL13Ra and IL4R. The IL-23 receptor is a heterodimer of IL23R and IL12Rb1. The TNFa receptor is, in embodiments, a heterodimer of TNFR1 and TNFR2. Without wishing to be bound by theory, in some instances of disease, for example in a cardiovascular disorder having an inflammatory component, it may be desirous to bind and clear a cytokine from circulation. In embodiments, this is achieved by administering an enucleated erythroid cell (e.g., a reticulocyte) that expresses one or more (e.g., 2 or 3) of the receptors for the target molecule simultaneously. A table of cytokines and their receptors is provided herein as Table 1. In some embodiments the agents are antibody molecules that bind cytokines, e.g., one or more cytokines of Table 1. In some embodiments, an enucleated erythroid cell comprises one or more (e.g., 2, 3, 4, 5, or more) cytokine receptor subunits from Table 1 or cytokine-binding variants or fragments thereof. In some embodiments, an enucleated erythroid cell comprises two or three (e.g., all) cytokine receptor subunits from a single row of Table 1 or cytokine-binding variants or functional fragments thereof. The cytokine receptors can be present on the surface of the erythroid cell. The expressed receptors typically have the wild type human receptor sequence or a variant or fragment thereof that is able to bind and sequester its target ligand. In embodiments, two or more cytokine receptor subunits are linked to each other, e.g., as a fusion protein.

TABLE 1
Cytokines and Receptors

| Name | Cytokine Receptor(s)(Da) and Form |
|---|---|
| Interleukins | |
| IL-1-like | |
| IL-1α | CD121a, CDw121b |
| IL-1β | CD121a, CDw121b |
| IL-1RA | CD121a |
| IL-18 | IL-18Rα, β |
| Common g chain (CD132) | |
| IL-2 | CD25, 122, 132 |
| IL-4 | CD124, 213a13, 132 |
| IL-7 | CD127, 132 |
| IL-9 | IL-9R, CD132 |
| IL-13 | CD213a1, 213a2, |
| IL-15 | IL-15Ra, CD122, 132 |
| Common b chain (CD131) | |
| IL-3 | CD123, CDw131 |
| IL-5 | CDw125, 131 |
| Also related | |
| GM-CSF | CD116, CDw131 |
| IL-6-like | |
| IL-6 | CD126, 130 |
| IL-11 | IL-11Ra, CD130 |
| Also related | |
| G-CSF | CD114 |
| IL-12 | CD212 |
| LIF | LIFR, CD130 |
| OSM | OSMR, CD130 |
| IL-10-like | |
| IL-10 | CDw210 |
| IL-20 | IL-20Rα, β |
| Others | |
| IL-14 | IL-14R |
| IL-16 | CD4 |
| IL-17 | CDw217 |
| Interferons | |
| IFN-α | CD118 |
| IFN-β | CD118 |
| IFN-γ | CDw119 |
| TNF | |
| CD154 | CD40 |
| LT-β | LTβR |
| TNF-α | CD120a, b |
| TNF-β (LT-α) | CD120a, b |
| 4-1BBL | CD137 (4-1BB) |
| APRIL | BCMA, TACI |
| CD70 | CD27 |
| CD153 | CD30 |
| CD178 | CD95 (Fas) |
| GITRL | GITR |
| LIGHT | LTbR, HVEM |
| OX40L | OX40 |
| TALL-1 | BCMA, TACI |
| TRAIL | TRAILR1-4 |
| TWEAK | Apo3 |
| TRANCE | RANK, OPG |
| TGF-β | |
| TGF-β1 | TGF-βR1 |
| TGF-β2 | TGF-βR2 |
| TGF-β3 | TGF-βR3 |
| Miscellaneous hematopoietins | |
| Epo | EpoR |
| Tpo | TpoR |
| Flt-3L | Flt-3 |
| SCF | CD117 |
| M-CSF | CD115 |
| MSP | CDw136 |

An enucleated erythroid cell can comprise a first exogenous polypeptide that interacts with a target and a second exogenous polypeptide (e.g., a protease) that modifies the target.

In embodiments, an effective amount of the enucleated erythroid cells comprising a first exogenous polypeptide and a second exogenous polypeptide is less than (e.g., less by 10%, 20%, 30%, 40%, 50%, 60%, 70%, 80%, 90%, 95%, 98%, 99%, 99.5%, 99.9%, or 99.99%) an effective amount of otherwise similar enucleated erythroid cells that lack the first exogenous polypeptide or lack the second exogenous polypeptide. In embodiments, the preselected amount is an effective dose or an in vitro effective amount of enucleated erythroid cells. In embodiments, the preselected amount (e.g., in vitro effective amount) is an amount that is effective in an assay, e.g., to convert at least 10%, 20%, 30%, 40%, 50%, 60%, 70%, 80%, or 90% of substrate into produce in a preselected amount of time, e.g., 1, 2, 3, 4, 5, or 6 hours. In embodiments, the preselected amount (e.g., in vitro effective amount) is effective to cleave or enzymatically convert at least 50% of a target (e.g., metabolite, amino acid, clotting factor, pro-inflammatory cytokine, or complement factor) in 5 hours. The assay may measure, e.g., reduction in levels of soluble, unmodified (e.g., non-cleaved) target in a solution.

In embodiments, the reference value for targets is the number of targets in the peripheral blood of the subject at the time of administration. In embodiments (e.g., embodiments involving an in vitro effective amount of cells) the reference value for targets is the number of targets in a reaction mixture for an assay.

First Exogenous Polypeptide

In embodiments, the first exogenous polypeptide can bind a target.

In embodiments, the first exogenous polypeptide comprises a binding domain (e.g., a domain that binds the target) and a membrane anchor domain (e.g., a transmembrane domain, e.g., type I or type II red blood cell transmembrane domain). In embodiments, the membrane anchor domain is C-terminal or N-terminal of the binding domain. In embodiments, the transmembrane domain comprises GPA or a transmembrane portion thereof, e.g., as set out in SEQ ID NO: 33 herein or a transmembrane portion thereof, or a polypeptide having at least 70%, 75%, 80%, 85%, 90%, 95%, 96%, 97%, 98%, 99% identity to any of the foregoing. In embodiments, the GPA polypeptide is C-terminal of the binding domain.

In embodiments, the first exogenous polypeptide comprises an address moiety or targeting moiety described in WO2007030708, e.g., in pages 34-45 therein, which application is herein incorporated by reference in its entirety.

Other examples of proteins that can be suitably adapted for use as the first exogenous polypeptide include ligand binding domains of receptors, such as where the target is the receptor ligand. Conversely, the first exogenous polypeptide can comprise a receptor ligand where the target is the receptor. A target ligand can be a polypeptide or a small molecule ligand.

In a further embodiment, a first exogenous polypeptide may comprise a domain derived from a polypeptide that has an immunoglobulin-like fold, such as the 10th type III domain of human fibronectin (“Fn3”). See U.S. Pat. Nos. 6,673,901; 6,462,189. Fn3 is small (about 95 residues), monomeric, soluble and stable. It does not have disulfide bonds which permit improved stability in reducing environments. The structure may be described as a beta-sandwich similar to that of Ab VH domain except that Fn3 has seven beta-strands instead of nine. There are three loops on each end of Fn3; and the positions of three of these loops correspond to those of CDR1, 2 and 3 of the VH domain. The 94 amino acid Fn3 sequence is:

(SEQ ID NO: 34)
VSDVPRDLEWAATPTSLLISWDAPAVTVRYYRITYGETGGNSPVQEFTVPG
SKSTATISGLKPGVDYTITGYAVTGRGDSPASSKPISINYRT

The amino acid positions of the CDR-like loops will be defined as residues 23-30 (BC Loop), 52-56 (DE Loop) and 77-87 (FG Loop). Accordingly, one or more of the CDR-like loops may be modified or randomized, to generate a library of Fn3 binding domains which may then be screened for binding to a desired address binding site. See also PCT Publication WO0232925. Fn3 is an example of a large subfamily of the immunoglobulin superfamily (IgSF). The Fn3 family includes cell adhesion molecules, cell surface hormone and cytokine receptors, chaperonin, and carbohydrate-binding domains, all of which may also be adapted for use as binding agents. Additionally, the structure of the DNA binding domains of the transcription factor NF-kB is also closely related to the Fn3 fold and may also be adapted for use as a binding agent. Similarly, serum albumin, such as human serum albumin contains an immunoglobulin-like fold that can be adapted for use as a targeting moiety.

In still other embodiments, the first exogenous polypeptide can comprise an engineered polypeptide sequence that was selected, e.g., synthetically evolved, based on its kinetics and selectivity for binding to the address site. In embodiments, the sequence of the first exogenous polypeptide is designed using a screen or selection method, e.g., by phage display or yeast two-hybrid screen.

In some embodiments, the first exogenous polypeptide comprises a peptide ligand for a soluble receptor (and optionally the target comprises a soluble receptor), a synthetic peptide that binds a target, a complement regulatory domain (and optionally the target comprises a complement factor), or a ligand for a cell surface receptor (and optionally the target comprises the cell surface receptor).

Second Exogenous Polypeptide (e.g., Protease)

In embodiments, the second exogenous polypeptide (which modifies the target) is a factor set out in Table 5. In some embodiments, the protease is a protease set out in Table 5. In embodiments, the protease is a bacterial protease, a human protease, or a plant protease, or a fragment or variant thereof.

In embodiments, the second exogenous polypeptide (which modifies the target) is a protease. Exemplary proteases include those classified as Aminopeptidases; Dipeptidases; Dipeptidyl-peptidases and tripeptidyl peptidases; Peptidyl-dipeptidases; Serine-type carboxypeptidases; Metallocarboxypeptidases; Cysteine-type carboxypeptidases; Omegapeptidases; Serine proteinases; Cysteine proteinases; Aspartic proteinases; Metalloproteinases; or Proteinases of unknown mechanism.

Aminopeptidases include cytosol aminopeptidase (leucyl aminopeptidase), membrane alanyl aminopeptidase, cystinyl aminopeptidase, tripeptide aminopeptidase, prolyl aminopeptidase, arginyl aminopeptidase, glutamyl aminopeptidase, x-pro aminopeptidase, bacterial leucyl aminopeptidase, thermophilic aminopeptidase, clostridial aminopeptidase, cytosol alanyl aminopeptidase, lysyl aminopeptidase, x-trp aminopeptidase, tryptophanyl aminopeptidase, methionyl aminopeptidase, d-stereospecific aminopeptidase, and aminopeptidase. Dipeptidases include x-his dipeptidase, x-arg dipeptidase, x-methyl-his dipeptidase, cys-gly dipeptidase, glu-glu dipeptidase, pro-x dipeptidase, x-pro dipeptidase, met-x dipeptidase, non-stereospecific dipeptidase, cytosol non-specific dipeptidase, membrane dipeptidase, and beta-ala-his dipeptidase. Dipeptidyl-peptidases and tripeptidyl peptidases include dipeptidyl-peptidase I, dipeptidyl-peptidase II, dipeptidyl peptidase III, dipeptidyl-peptidase IV, dipeptidyl-dipeptidase, tripeptidyl-peptidase I, and tripeptidyl-peptidase II. Peptidyl-dipeptidases include peptidyl-dipeptidase A and peptidyl-dipeptidase B. Serine-type carboxypeptidases include lysosomal pro-x carboxypeptidase, serine-type D-ala-D-ala carboxypeptidase, carboxypeptidase C, and carboxypeptidase D. Metallocarboxypeptidases include carboxypeptidase A, carboxypeptidase B, lysine(arginine) carboxypeptidase, gly-X carboxypeptidase, alanine carboxypeptidase, muramoylpentapeptide carboxypeptidase, carboxypeptidase H, glutamate carboxypeptidase, carboxypeptidase M, muramoyltetrapeptide carboxypeptidase, zinc D-ala-D-ala carboxypeptidase, carboxypeptidase A2, membrane pro-x carboxypeptidase, tubulinyl-tyr carboxypeptidase, and carboxypeptidase T. Omegapeptidases include acylaminoacyl-peptidase, peptidyl-glycinamidase, pyroglutamyl-peptidase I, beta-aspartyl-peptidase, pyroglutamyl-peptidase II, n-formylmethionyl-peptidase, pteroylpoly-[gamma]-glutamate carboxypeptidase, gamma-glu-X carboxypeptidase, and acylmuramoyl-ala peptidase. Serine proteinases include chymotrypsin, chymotrypsin C, metridin, trypsin, thrombin, coagulation factor Xa, plasmin, enteropeptidase, acrosin, alpha-lytic protease, glutamyl, endopeptidase, cathepsin G, coagulation factor VIIa, coagulation factor IXa, cucumisi, prolyl oligopeptidase, coagulation factor XIa, brachyurin, plasma kallikrein, tissue kallikrein, pancreatic elastase, leukocyte elastase, coagulation factor XIIa, chymase, complement component c1r55, complement component c1s55, classical-complement pathway c3/c5 convertase, complement factor I, complement factor D, alternative-complement pathway c3/c5 convertase, cerevisin, hypodermin C, lysyl endopeptidase, endopeptidase la, gamma-reni, venombin AB, leucyl endopeptidase, tryptase, scutelarin, kexin, subtilisin, oryzin, endopeptidase K, thermomycolin, thermitase, endopeptidase SO, T-plasminogen activator, protein C, pancreatic endopeptidase E, pancreatic elastase II, IGA-specific serine endopeptidase, U-plasminogen, activator, venombin A, furin, myeloblastin, semenogelase, granzyme A or cytotoxic T-lymphocyte proteinase 1, granzyme B or cytotoxic T-lymphocyte proteinase 2, streptogrisin A, treptogrisin B, glutamyl endopeptidase II, oligopeptidase B, limulus clotting factor C, limulus clotting factor, limulus clotting enzyme, omptin, repressor lexa, bacterial leader peptidase I, and togavirin, flavirin. Cysteine proteinases include cathepsin B, papain, ficin, chymopapain, asclepain, clostripain, streptopain, actinide, cathepsin 1, cathepsin H, calpain, cathepsin T, glycyl, endopeptidase, cancer procoagulant, cathepsin S, picornain 3C, picornain 2A, caricain, ananain, stem bromelain, fruit bromelain, legumain, histolysain, and interleukin 1-beta converting enzyme. Aspartic proteinases include pepsin A, pepsin B, gastricsin, chymosin, cathepsin D, neopenthesin, renin, retropepsin, pro-opiomelanocortin converting enzyme, aspergillopepsin I, aspergillopepsin II, penicillopepsin, rhizopuspepsin, endothiapepsin, mucoropepsin, candidapepsin, saccharopepsin, rhodotorulapepsin, physaropepsin, acrocylindropepsin, polyporopepsin, pycnoporopepsin, scytalidopepsin A, scytalidopepsin B, xanthomonapepsin, cathepsin E, barrierpepsin, bacterial leader peptidase I, pseudomonapepsin, and plasmepsin. Metalloproteinases include atrolysin A, microbial collagenase, leucolysin, interstitial collagenase, neprilysin, envelysin, IgA-specific metalloendopeptidase, procollagen N-endopeptidase, thimet oligopeptidase, neurolysin, stromelysin 1, meprin A, procollagen C-endopeptidase, peptidyl-lys metalloendopeptidase, astacin, stromelysin 2, matrilysin gelatinase, aeromonolysin, pseudolysin, thermolysin, bacillolysin, aureolysin, coccolysin, mycolysin, beta-lytic metalloendopeptidase, peptidyl-asp metalloendopeptidase, neutrophil collagenase, gelatinase B, leishmanolysin, saccharolysin, autolysin, deuterolysin, serralysin, atrolysin B, atrolysin C, atroxase, atrolysin E, atrolysin F, adamalysin, horrilysin, ruberlysin, bothropasin, bothrolysin, ophiolysin, trimerelysin I, trimerelysin II, mucrolysin, pitrilysin, insulysin, O-syaloglycoprotein endopeptidase, russellysin, mitochondrial, intermediate, peptidase, dactylysin, nardilysin, magnolysin, meprin B, mitochondrial processing peptidase, macrophage elastase, choriolysin, and toxilysin. Proteinases of unknown mechanism include thermopsin and multicatalytic endopeptidase complex. In embodiments, the second exogenous polypeptide comprises a fragment or variant of any of the foregoing.

In embodiments, the second exogenous polypeptide comprises an IdeS polypeptide. In some embodiments, the IdeS polypeptide comprises the sequence set out below as SEQ ID NO: 35 or a proteolytically active fragment of the sequence of SEQ ID NO: 35 (e.g., a fragment of at least 100, 150, 200, 250, or 300 amino acids) or a sequence having at least 70%, 75%, 80%, 85%, 90%, 95%, 96%, 97%, 98%, 99% identity to any of the foregoing. In some embodiments involving nucleic acids, the nucleic acid encodes an IdeS polypeptide having the sequence set out below as SEQ ID NO: 35, or a proteolytically active fragment thereof, or a sequence having at least 70%, 75%, 80%, 85%, 90%, 95%, 96%, 97%, 98%, 99% identity to any of the foregoing.

IdeS Polypeptide:

(SEQ ID NO: 35)
DSFSANQEIRYSEVTPYHVTSVWTKGVTPPAKFTQGEDVFHAPYVANQGWY
DITKTFNGKDDLLCGAATAGNMLHWWFDQNKEKIEAYLKKHPDKQKIMFGD
QELLDVRKVINTKGDQTNSELFNYFRDKAFPGLSARRIGVMPDLVLDMFIN
GYYLNVYKTQTTDVNRTYQEKDRRGGIFDAVFTRGDQSKLLTSRHDFKEKN
LKEISDLIKKELTEGKALGLSHTYANVRINHVINLWGADFDSNGNLKAIYV
TDSDSNASIGMKKYFVGVNSAGKVAISAKEIKEDNIGAQVLGLFTLSTGQD
SWNQTN

In embodiments, the second exogenous polypeptide comprises a modifier domain (e.g., a protease domain, e.g., an IdeS polypeptide) and a membrane anchor domain (e.g., a transmembrane domain, e.g., type I or type II red blood cell transmembrane domain). In embodiments, the membrane anchor domain is C-terminal or N-terminal of the modifier (e.g., protease) domain. In embodiments, the transmembrane domain comprises GPA or a transmembrane portion thereof. In embodiments, the GPA polypeptide has a sequence of:

(SEQ ID NO: 33)
LSTTEVAMHTSTSSSVTKSYISSQTNDTHKRDTYAATPRAHEVSEISVRTV
YPPEEETGERVQLAHHFSEPEITLIIFGVMAGVIGTILLISYGIRRLIKKS
PSDVKPLPSPDTDVPLSSVEIENPETSDQ

or a transmembrane portion thereof, or a polypeptide having at least 70%, 75%, 80%, 85%, 90%, 95%, 96%, 97%, 98%, 99% identity to any of the foregoing. In embodiments, the GPA polypeptide is C-terminal of the modifier (e.g., protease) domain.

In some embodiments, a linker is disposed between the IdeS polypeptide and the transmembrane polypeptide, e.g., a glycine-serine linker, e.g., a linker comprising a sequence of GGSGGSGG (SEQ ID NO: 36) and/or GGGSGGGS (SEQ ID NO: 37).

In some embodiments, the exogenous polypeptide, e.g., the second exogenous polypeptide, e.g., a protease, e.g., IdeS polypeptide, comprises a leader sequence, e.g., a GPA leader sequence, e.g., MYGKIIFVLLLSEIVSISA (SEQ ID NO: 38).

In some embodiments, the exogenous polypeptide, e.g., the second exogenous polypeptide further comprises a tag, e.g., an HA tag or a FLAG tag.

In some embodiments, the protease (e.g., immunoglobulin degrading enzyme, e.g., immunoglobulin-G degrading enzyme, e.g., IdeS) cleaves an immunoglobulin at a hinge region, a CH2 region, or between a hinge and CH2 region. In embodiments, the protease cleaves an immunoglobulin at one of the sequences below, e.g., between the two italicized glycines or the italicized alanine and glycine in the sequences below.

Human IgG1 Hinge/CH2 Sequence
(SEQ ID NO: 39)
CPPCPAPELLGGPSVF
Human IgG2 Hinge/CH2 Sequence
(SEQ ID NO: 40)
CPPCPAPPVAGPSVF
Human IgG3 Hinge/CH2 Sequence
(SEQ ID NO: 41)
CPRCPAPELLGGPSVF
Human IgG4 Hinge/CH2 Sequence
(SEQ ID NO: 42)
AHHAQAPEFLGGPSVF

In embodiments, the protease (e.g., a bacterial protease) cleaves IgG, e.g., IdeS or IgA protease.

In embodiments, the protease (e.g., a papain family protease, e.g., papain) cleaves an immunoglobulin between the Fc and Fab regions, e.g., a histidine-threonine bond between positions 224 and 225 of the heavy chain and/or a glutamic acid-leucine bond between positions 233 and 234 of the heavy chain.

In embodiments, the protease or other modifier acts on a target listed in Table 5 or Table 6.

In embodiments, the protease or other modifier acts on (e.g., inactivates or inhibits) a TNF molecule (such as TNF-alpha), e.g., in a subject having a cardiovascular disease. For instance, the first exogenous polypeptide can comprise a TNF-alpha binding moiety such as an anti-TNF-alpha antibody, and the second exogenous polypeptide can comprise a protease that cleaves TNF-alpha, e.g., MT1-MMP, MMP12, tryptase, MT2-MMP, elastase, MMP7, chymotrypsin, or trypsin, or active variants or fragments thereof.

In embodiments, the second exogenous polypeptide comprises a catalytic moiety described in WO2007030708, e.g., in pages 45-46 therein, which application is herein incorporated by reference in its entirety.

The second exogenous polypeptide can comprise a moiety capable of acting on a target to induce a chemical change, thereby modulate its activity, e.g., a moiety capable of catalyzing a reaction within a target. The second exogenous polypeptide can comprise a naturally occurring enzyme, an active (e.g., catalytically active) fragment thereof, or an engineered enzyme, e.g., a protein engineered to have an enzymatic activity, such as a protein designed to contain a serine protease active motif. A catalytic domain of a second exogenous polypeptide may comprise the arrangement of amino acids that are effective to induce the desired chemical change in the target. They may be N-terminal or C-terminal truncated versions of natural enzymes, mutated versions, zymogens, or complete globular domains.

The second exogenous polypeptide can comprise an enzymatically active site that alone is promiscuous, binding with a cleavage site it recognizes on many different biomolecules, and may have relatively poor reaction kinetics. In embodiments, the first exogenous polypeptide supplies or improves specificity by increasing the local concentration of target near the second exogenous polypeptide.

The second exogenous polypeptide can, in embodiments, modify the target so that it is recognized and acted upon by another enzyme (e.g., an enzyme that is already present in a subject). In an embodiment, the second exogenous polypeptide comprises a moiety that alters the structure of the target so that its activity is inhibited or upregulated. Many naturally occurring enzymes activate other enzymes, and these can be exploited in accordance with the compositions and methods described herein.

The second exogenous polypeptide can comprise a protease, a glycosidase, a lipase, or other hydrolases, an amidase (e.g., N-acetylmuramoyl-L-alanine amidase, PGRP-L amidase), or other enzymatic activity, including isomerases, transferases (including kinases), lyases, oxidoreductases, oxidases, aldolases, ketolases, glycosidases, transferases and the like. In embodiments, the second exogenous polypeptide comprises human lysozyme, a functional portion of a human lysozyme, a human PGRP-L, a functional portion of a human PGRP-L, a phospholipase A2, a functional portion of a phospholipase A2, or a matrix metalloproteinase (MMP) extracellular enzyme such as MMP-2 (gelatinase A) or MMP-9 (gelatinase B).

In embodiments, the second exogenous polypeptide is a serine proteinase, e.g., of the chymotrypsin family which includes the mammalian enzymes such as chymotrypsin, trypsin or elastase or kallikrein, or the substilisin family which includes the bacterial enzymes such as subtilisin. The general three-dimensional structure is different in the two families but they have the same active site geometry and catalysis proceeds via the same mechanism. The serine proteinases exhibit different substrate specificities which are related to amino acid substitutions in the various enzyme subsites interacting with the substrate residues. Three residues which form the catalytic triad are important in the catalytic process: His-57, Asp-102 and Ser-195 (chymotrypsinogen numbering).

In embodiments, the second exogenous polypeptide is a cysteine proteinase which includes the plant proteases such as papain, actinidin or bromelain, several mammalian lysosomal cathepsins, the cytosolic calpains (calcium-activated), and several parasitic proteases (e.g., Trypanosoma, Schistosoma). Papain is the archetype and the best studied member of the family. Like the serine proteinases, catalysis proceeds through the formation of a covalent intermediate and involves a cysteine and a histidine residue. The essential Cys-25 and His-159 (papain numbering) play the same role as Ser-195 and His-57 respectively. The nucleophile is a thiolate ion rather than a hydroxyl group. The thiolate ion is stabilized through the formation of an ion pair with neighboring imidazolium group of His-159. The attacking nucleophile is the thiolate-imidazolium ion pair in both steps and then a water molecule is not required.

In embodiments, the second exogenous polypeptide is an aspartic proteinase, most of which belong to the pepsin family. The pepsin family includes digestive enzymes such as pepsin and chymosin as well as lysosomal cathepsins D, processing enzymes such as renin, and certain fungal proteases (penicillopepsin, rhizopuspepsin, endothiapepsin). A second family comprises viral proteinases such as the protease from the AIDS virus (HIV) also called retropepsin. In contrast to serine and cysteine proteinases, catalysis by aspartic proteinases does not involve a covalent intermediate, though a tetrahedral intermediate exists. The nucleophilic attack is achieved by two simultaneous proton transfers: one from a water molecule to the dyad of the two carboxyl groups and a second one from the dyad to the carbonyl oxygen of the substrate with the concurrent CO-NH bond cleavage. This general acid-base catalysis, which may be called a “push-pull” mechanism leads to the formation of a non-covalent neutral tetrahedral intermediate.

In embodiments, the second exogenous polypeptide is a metalloproteinase, which can be found in bacteria, fungi as well as in higher organisms. They differ widely in their sequences and their structures but the great majority of enzymes contain a zinc (Zn) atom which is catalytically active. In some cases, zinc may be replaced by another metal such as cobalt or nickel without loss of the activity. Bacterial thermolysin has been well characterized and its crystallographic structure indicates that zinc is bound by two histidines and one glutamic acid. Many enzymes contain the sequence HEXXH, which provides two histidine ligands for the zinc whereas the third ligand is either a glutamic acid (thermolysin, neprilysin, alanyl aminopeptidase) or a histidine (astacin). Other families exhibit a distinct mode of binding of the Zn atom. The catalytic mechanism leads to the formation of a non-covalent tetrahedral intermediate after the attack of a zinc-bound water molecule on the carbonyl group of the scissile bond. This intermediate is further decomposed by transfer of the glutamic acid proton to the leaving group.

In embodiments, the second exogenous polypeptide comprises an isomerase (e.g., an isomerase that breaks and forms chemical bonds or catalyzes a conformational change). In embodiments, the isomerase is a racemase (e.g., amino acid racemase), epimerase, cis-trans isomerase, intramolecular oxidoreductase, intramolecular transferase, or intramolecular lyase.

In embodiments, the second exogenous protease comprises a chaperone, or an active variant or fragment thereof. For instance, the chaperone can be a general chaperone (e.g., GRP78/BiP, GRP94, GRP170), a lectin chaperone (e.g., calnexin or calreticulin), a non-classical molecular chaperone (e.g., HSP47 or ERp29), a folding chaperone (e.g., PDI, PPI, or ERp57), a bacterial or archaeal chaperone (e.g., Hsp60, GroEL/GroES complex, Hsp70, DnaK, Hsp90, HtpG, Hsp100, Clp family (e.g., ClpA and ClpX), Hsp104). In embodiments, the enucleated erythrocyte comprises a co-chaperone, or an active variant or fragment thereof, e.g., immunophilin, Stil, p50 (Cdc37), or Aha1. In embodiments, the molecular chaperone is a chaperonin.

Candidates for the second exogenous protein (which modifies a target) can be screened based on their activity. Depending on the specific activity of each molecule being tested, an assay appropriate for that molecule can be used. For example, if the second exogenous protein is a protease, the assay used to screen the protease can be an assay to detect cleavage products generated by the protease, e.g., a chromatography or gel electrophoresis based assay.

In an example, the second exogenous polypeptide may have kinase activity. An assay for kinase activity could measure the amount of phosphate that is covalently incorporated into the target of interest. For example, the phosphate incorporated into the target of interest could be a radioisotope of phosphate that can be quantitated by measuring the emission of radiation using a scintillation counter.

Targets (e.g., Complement Pathway Factors, Clotting Factors, and Amino Acids) and Indications

In embodiments, the target is a target listed in Table 5 or Table 6.

In embodiments, the target is a complement factor, e.g., a factor that acts in the classical complement pathway or the alternative complement pathway. In embodiments, the complement factor is a pro-protein or an activated (e.g., cleaved) protein. In embodiments, the complement factor comprises C1, C2a, C4b, C3, C3a, C3b, C5, C5a, C5b, C6, C7, C8, or C9. In embodiments, the second exogenous polypeptide cleaves the complement factor. In embodiments, the second exogenous polypeptide activates the complement factor, e.g., to promote an immune response. For instance, the second exogenous polypeptide may comprise a complement control protein and/or complement activation family protein such as Factor H or Factor I (which promote C3b cleavage), or a fragment or variant thereof. In embodiments, the second exogenous polypeptide inactivates the complement factor, e.g., by cleaving it to yield one or more inactive fragments, e.g., to reduce an unwanted immune response, e.g., to treat an autoimmune or inflammatory disease. In embodiments, the first exogenous polypeptide binds a complement factor (e.g., binds complement factor C3b) and the second exogenous polypeptide cleaves the complement factor, e.g., to iC3b. For instance, the first exogenous polypeptide could comprise Factor H or CR1 or a fragment or variant thereof and the second exogenous polypeptide could comprise Factor I or a fragment or variant thereof.

Engineered erythroid cells described herein can also be used to treat a subject that has antibodies against a drug. The erythroid cell can reduce levels of anti-drug antibodies in a subject, e.g., a subject that produces unwanted anti-FVIII antibody in response to prior treatment with Factor VIII, and can optionally further comprise a therapeutic protein that treats the disease. For instance, the erythroid cell comprises a first exogenous polypeptide that binds a target, e.g., wherein the target is an anti-drug antibody. The erythroid cell can further comprise a second exogenous polypeptide (e.g., a protease) that inactivates, e.g., cleaves the target. The erythroid cell may optionally further comprise a third exogenous polypeptide, e.g., a therapeutic protein that treats the same disease as the prior therapeutic to which the subject developed anti-drug antibodies, e.g., a therapeutic protein which is the same as or different from the prior therapeutic to which the subject developed anti-drug antibodies. In embodiments, the subject comprises anti-drug antibodies against erythropoietin, an anti-TNF antibody molecule (adalimumab or infliximab), an anti-EGFR antibody (e.g., cetuximab), an anti-CD20 antibody molecule, insulin, an anti-alpha4 integrin antibody molecule (e.g., natalizumab), or an interferon, e.g., IFNβ1a or IFNβ1b. In embodiments, the first polypeptide comprises an anti-MAdCAM-1 antibody molecule and the second polypeptide comprises LysC or LysN (which can cleave MAdCAM-1 at 1 or more (e.g., 2, 3, or 4 sites), or a fragment or variant thereof having protease activity, e.g., wherein the target tissue is inflamed tissue, e.g., inflamed cardiac tissue or vasculature. In some embodiments, the patient may be tested for the presence of anti-drug antibodies, e.g., for the presence of neutralizing anti-drug antibodies, before, during and/or after administration of the engineered erythroid cells described herein.

In embodiments, the target is a clotting factor. In embodiments, the clotting factor is a pro-protein or an activated (e.g., cleaved) protein. In embodiments, the target comprises Tissue Factor, Factor V, Factor VII, Factor VIII, Factor IX, Factor X, Factor XI, Factor XIII, thrombin, or fibrinogen. In embodiments, the second exogenous polypeptide cleaves the clotting factor. In embodiments, the second exogenous polypeptide activates the clotting factor, e.g., to allow clotting in a subject having a clotting deficiency disease such as hemophilia (e.g., hemophilia type A, hemophilia type B, or hemophilia Type C), von Willebrand disease, Factor II deficiency, Factor V deficiency, Factor VII deficiency, Factor X deficiency, or Factor XII deficiency. For instance, the second exogenous polypeptide may comprise an activated clotting factor, e.g., an activated form of Tissue Factor, Factor V, Factor VII, Factor VIII, Factor IX, Factor X, Factor XI, Factor XIII, thrombin, or fibrinogen. In some embodiments, the second exogenous polypeptide comprises a clotting factor and the target comprises its substrate; the second exogenous polypeptide comprises Tissue Factor and the target comprises Factor VII; the second exogenous polypeptide comprises TF-VIIa and/or Factor IXa and the target comprises Factor X; the second exogenous polypeptide comprises Factor XIa and the target comprises Factor IX; the second exogenous polypeptide comprises TF-VIIa and the target comprises Factor IX; the second exogenous polypeptide comprises Factor VIIIa and the target comprises Factor X; the second exogenous polypeptide comprises Factor XI, Factor VIII, or Factor V and the target comprises thrombin; the second exogenous polypeptide comprises Factor Va or Xa and the target comprises prothrombin; the second exogenous polypeptide comprises thrombin and the target comprises fibrinogen or Factor XIII In embodiments, the second exogenous polypeptide inactivates the clotting factor, e.g., by cleaving it to yield one or more inactive fragments, e.g., to reduce unwanted clotting, e.g., in a subject having or at risk of developing a blood clot. In embodiments, the subject has, or is at risk of developing, thrombophilia, pulmonary embolism, or stroke. For example, in some embodiments, the second exogenous polypeptide comprises a plasminogen activator (e.g., urokinase or TPA) and the target comprises plasminogen.

In one embodiment the disease or condition is hemophilia A, the target is thrombin (factor II a) or factor X, the first exogenous polypeptide binds the target (e.g., is an antibody for the target), and the second exogenous polypeptide comprises factor VIII or fragment thereof.

In one embodiment the disease or condition is hemophilia B, the target is factor XIa or factor X, the first exogenous polypeptide binds the target (e.g., is an antibody for the target), and the second exogenous polypeptide comprises factor IX or fragment thereof.

In one embodiment the disease or condition is thrombotic thrombocytopenic purpura, the target is ultra-large von Willebrand factor (ULVWF), the first exogenous polypeptide binds the target (e.g., is an antibody for the target), and the second exogenous polypeptide comprises ADAMTS13 or fragment thereof.

Agent-Synergistic Configurations

When two or more agents (e.g., polypeptides) are agent-synergistic, the agents act on two or more different targets within a single pathway. In an embodiment, the action of the two or more agents together is greater than the action of any of the individual agents. For example, the first and second polypeptides are ligands for cellular receptors that signal to the same downstream target. For example, the first exogenous polypeptide comprises a ligand for a first target cellular receptor, and the second exogenous polypeptide comprises a ligand for a second target cellular receptor, e.g., which first and second target cellular receptors signal to the same downstream target. In embodiments, the first exogenous polypeptide acts on the first target and the second exogenous polypeptide acts on the second target simultaneously, e.g., there is some temporal overlap in binding of the first exogenous polypeptide to the first target and binding of the second exogenous polypeptide to the second target. In some embodiments the simultaneous action generates a synergistic response of greater magnitude than would be expected when either target is acted on alone or in isolation.

In an embodiment, the first and second polypeptides are ligands for a first cellular receptor and a second cellular receptor that mediates apoptosis. In an embodiment the agents comprise two or more TRAIL receptor ligands, e.g., wild-type or mutant TRAIL polypeptides, or antibody molecules that bind TRAIL receptors, and induce apoptosis in a target cell, e.g., a cell that promotes inflammation associated with a cardiovascular disease. In some embodiments, a erythroid cell comprising TRAIL receptor ligands further comprises a targeting moiety, e.g., a targeting moiety described herein. In an embodiment the first target and the second target interacts with the same substrate, e.g., a substrate protein. In an embodiment the first target and the second target interact with different substrates.

TRAIL (TNF-related apoptosis inducing ligand) is a member of the TNF family that induces apoptosis. TRAIL has at least two receptors, TRAIL R1 and TRAIL R2. TRAIL receptor agonists, e.g., mutants of TRAIL that bind one or more of the receptors, or antibody molecules that bind one or both of TRAIL R1 or TRAIL R2 (see, e.g. Gasparian et al., Apoptosis 2009 Jun. 14(6), Buchsbaum et al. Future Oncol 2007 Aug. 3(4)), have been developed as a clinical therapy for a wide range of cancers. Clinical trials of TRAIL receptor agonists have failed for, among other reasons, the fact that many primary cancers are not sensitive to signaling through a single receptor but rather require engagement of both receptors to induce cytotoxicity (Marconi et al., Cell Death and Disease (2013) 4, e863). In one embodiment the agents expressed on the engineered erythroid cell are single receptor-specific TRAIL agonists that, in combination, enable the cell to engage and agonize both TRAIL receptors simultaneously, thus leading to a synergistic induction of apoptosis of a target cell. Thus, in some embodiments, the enucleated erythroid cell comprises on its surface a first polypeptide that binds TRAIL R1 and a second polypeptide that binds TRAIL R2. In embodiments, each polypeptide has a Kd for TRAIL R1 or TRAIL R2 that is 2, 3, 4, 5, 10, 20, 50, 100, 200, or 500-fold stronger than the Kd for the other receptor. While not wishing to be bound by theory, in some embodiments an enucleated erythroid cell comprising a TRAIL R1-specific ligand and a TRAIL R2-specific ligand promote better heterodimerization of TRAIL R1 and TRAIL R2 than an enucleated erythroid cell comprising a ligand that binds to TRAIL R1 and TRAIL R2 with about the same affinity.

In some embodiments, one, two, or more of the exogenous polypeptides are members of the TNF superfamily. In some embodiments, the exogenous polypeptides bind to one or both of death receptors DR4 (TRAIL-R1) and DRS (TRAIL-R2). In some embodiments, the exogenous polypeptides bind to one or more of TNFRSF10A/TRAILR1, TNFRSF10B/TRAILR2, TNFRSF10C/TRAILR3, TNFRSF10D/TRAILR4, or TNFRSF11B/OPG. In some embodiments, the exogenous polypeptides activate one or more of MAPK8/JNK, caspase 8, and caspase 3.

In some embodiments, a TRAIL polypeptide is a TRAIL agonist having a sequence of any of SEQ ID NOS: 43-47 herein, or a sequence with at least 70%, 80%, 85%, 90%, 95%, 96%, 97%, 98%, or 99% identity thereto. Sequence identity is measured, e.g., by BLAST (Basic Local Alignment Search Tool). SEQ ID Nos. 43-47 are further described in Mohr et al. BMC Cancer (2015) 15:494), which is herein incorporated by reference in its entirety.

Soluble TRAIL variant DR4-1
SEQ ID NO: 43
MAMMEVQGGPSLGQTCVLIVIFTVLLQSLCVAVTYVYFTNELKQMQDKYSK
SGIACFLKEDDSYWDPNDEESMNSPCWQVKWQLRQLVRKMILRTSEETIST
VQEKQQNISPLVRERGPQRVAAHITGTRRRSNTLSSPNSKNEKALGRKINS
WESSRSGHSFLSNLHLRNGELVIHEKGFYYIYSQTYFRFQEEIKENTKNDK
QMVQYIYKYTSYPDPILLMKSARNSCWSKDAEYGLYSIYQGGIFELKENDR
IFVSVTNEHLIDMDHEASFFGAFLVG
Soluble TRAIL variant DR4-2
SEQ ID NO: 44
MAMMEVQGGPSLGQTCVLIVIFTVLLQSLCVAVTYVYFTNELKQMQDKYSK
SGIACELKEDDSYWDPNDEESMNSPCWQVKWQLRQLVRKMILRTSEETIST
VQEKQQNISPLVRERGPQRVAAHITGTRGRSNTLSSPNSKNEKALGRKINS
WESSRRGHSFLSNLHLRNGELVIHEKGFYYIYSQTYFRFQEEIKENTKNDK
QMVQYIYKYTSYPDPILLMKSARNSCWSKDAEYGLYSIYQGGIFELKENDR
IFVSVTNEHLIDMDHEASFFGAFLVG
Soluble TRAIL variant DR4-3
SEQ ID NO: 45
MAMMEVQGGPSLGQTCVLIVIFTVLLQSLCVAVTYVYFTNELKQMQDKYSK
SGIACFLKEDDSYWDPNDEESMNSPCWQVKWQLRQLVRKMILRTSEETIST
VQEKQQNISPLVRERGPQRVAAHITGTRRRSNTLSSPNSKNEKALGIKINS
WESSRRGHSFLSNLHLRNGELVIHEKGFYYIYSQTYFRFQEEIKENTKNDK
QMVQYIYKYTDYPDPILLMKSARNSCWSKDAEYGLYSIYQGGIFELKENDR
IFVSVTNEHLIDMDHEASFFGAFLVG
Soluble TRAIL variant DR5-1
SEQ ID NO: 46
MAMMEVQGGPSLGQTCVLIVIFTVLLQSLCVAVTYVYFTNELKQMQDKYSK
SGIACELKEDDSYWDPNDEESMNSPCWQVKWQLRQLVRKMILRTSEETIST
VQEKQQNISPLVRERGPQRVAAHITGTRGRSNTLSSPNSKNEKALGRKINS
WESSRSGHSFLSNLHLRNGELVIHEKGFYYIYSQTYFRFQEEIKENTKNDK
QMVQYIYKYTSYPDPILLMKSARNSCWSKDAEYGLYSIYQGGIFELKENDR
IFVSVTNEHLIDMHHEASFFGAFLVG
Soluble TRAIL variant DR5-2
SEQ ID NO: 47
MAMMEVQGGPSLGQTCVLIVIFTVLLQSLCVAVTYVYFTNELKQMQDKYSK
SGIACFLKEDDSYWDPNDEESMNSPCWQVKWQLRQLVRKMILRTSEETIST
VQEKQQNISPLVRERGPQRVAAHITGTRGRSNTLSSPNSKNEKALGRKINS
WESSRSGHSFLSNLHLRNGELVIHEKGFYYIYSQTYFRFQERIKENTKNDK
QMVQYIYKYTSYPDPILLMKSARNSCWSKDAEYGLYSIYQGGIFELKENDR
IFVSVTNEHLIDMHHEASFFGAFLVG

All combinations of the TRAIL receptor ligands are envisioned. In some embodiments, the first and second agents comprise SEQ ID NO: 43 and SEQ ID NO: 44; SEQ ID NO: 43 and SEQ ID NO: 45; SEQ ID NO: 434 and SEQ ID NO: 36; SEQ ID NO: 43 and SEQ ID NO: 47; SEQ ID NO: 44 and SEQ ID NO: 45; SEQ ID NO: 44 and SEQ ID NO: 46; SEQ ID NO: 44 and SEQ ID NO: 47; SEQ ID NO: 45 and SEQ ID NO: 46; SEQ ID NO: 45 and SEQ ID NO: 47; or SEQ ID NO: 45 and SEQ ID NO: 48, or a fragment or variant of any of the foregoing.

In some embodiments, the TRAIL receptor ligand comprises an antibody molecule. In embodiments, the antibody molecule recognizes one or both of TRAIL R1 and TRAIL R2. The antibody molecule may be, e.g., Mapatumumab (human anti-DR4 mAb), Tigatuzumab (humanized anti-DR5 mAb), Lexatumumab (human anti-DR5 mAb), Conatumumab (human anti-DR5 mAb), or Apomab (human anti-DR5 mAb), or a fragment or variant thereof, e.g., a variant having the same CDRs as any of the aforementioned antibodies, e.g., by the Chothia or Kabat definitions. In some embodiments, the enucleated erythroid cell comprises two or more (e.g., three, four, five, or more) different antibody molecules that bind a TRAIL receptor. In some embodiments, the enucleated erythroid cell comprises at least one antibody molecule that binds a TRAIL receptor and at least one TRAIL polypeptide.

In some embodiments, the agents are modulators of a multi-step pathway that act agent-synergistically by targeting upstream and downstream steps of the pathway, e.g., simultaneously. In one embodiment, the target pathway is the complement cascade, which has several parallel activation paths (classical, alternative, lectin pathways) and multiple auto-catalytic enzymes to enhance its potency in responding to infection and leading to membrane-attack complex formation (see, e.g. Bu et al., Clin Dev Immunol. 2012; 2012: 370426). Inhibitors of complement cascade exist, both as synthetic antibodies and peptides, that act on different levels of the cascade, e.g. anti-C5 (eculizumab) and anti-C3 (compstatin), and as endogenous proteins and polypeptides, e.g. CFH, CFI, CD46/MCP, CD55/DAF, CD59, and CR1. Non-enzymatic complement inhibitors include include Efb (extracellular fibrinogen-binding protein, e.g., from S. aureus, which binds C3b), Ehp (binds C3d and inhibits C3 conversion), SCIN (staphylococcal complement inhibitor, which stabilize C3 convertase into a non-functional state), CHIPS (chemotaxis inhibitory protein of S. aureus, which antagonizes C5a receptor), and SSL-7 (Staphyloccal superantigen-like protein-7, which binds C5). Enzymatic complement inhibitors include LysC, LysN, PaE, PaAP, 56 kDa protease from Serratia marcescens, C5a peptidases, Plasmin, SpeB, PrtH, Staphylokinase, and MMPs (see Table 1). The exogenous polypeptide can also comprise a fragment or variant of any of the complement inhibitors described herein. To treat diseases of complement over-activation it can be beneficial to inhibit the complement cascade, and it can be especially beneficial to intervene at two or more stages of the cascade to obtain a more potent inhibition. In some embodiments the agents expressed on the engineered erythroid cell are inhibitors of the complement cascade that act on different levels of the cascade, e.g. CFI and MCP, CD55 and CD59, or anti-C3 and anti-C5. In embodiments, an erythroid cell comprising complement inhibitors is used to treat a cardiac disease described herein.

In some embodiments, the enucleated erythroid cell comprises two or more agents that are anti-inflammatory. For instance, the agents can comprise an anti-TNFa antibody molecule (e.g., humira), an anti-IgE antibody molecule (e.g., Xolair), or a molecule that inhibits T cells (e.g., IL-10 or PD-L1; also see Table 1 and Table 3), or any combination thereof. In embodiments, one or more agents capture a factor such as a cytokine. In embodiments, one or more agents modulate immune cells. In embodiments, an erythroid cell comprising anti-inflammatory agents is used to treat a cardiac disease described herein.

In some embodiments, the enucleated erythroid cell comprises two or more agents to treat cardiovascular disease, e.g., hypercholesterolemia or hereditary angioedema. For instance, the agents can comprise an anti-PCSK9 antibody molecule, kallikrein inhibitor such as ecallantaide, or a fragment or variant thereof.

Multiplicative Configurations

When two or more agents (e.g., polypeptides) are multiplicative, a first agent acts on a first molecule that is part of a first pathway and a second agent acts on a second molecule that is part of a second pathway, which pathways act in concert toward a desired response.

In some embodiments, the desired response is downregulation of an inflammatory response, e.g., in a subject having a cardiovascular disease. In some embodiments, the agents inhibit multiple T cell activation pathways. In embodiments, one or more (e.g., 2, 3, 4, or 5 or more) T cell inhibition ligands comprise an inhibiting variant (e.g., fragment) of a ligand of Table 2. In embodiments, one or more (e.g., 2, 3, 4, or 5 or more) T cell inhibition ligands comprise an inhibitory antibody molecule that binds a target receptor of Table 2 or a T-cell inhibiting variant (e.g., fragment) thereof. In embodiments, these proteins signal through complementary activation pathways. In some embodiments the ligands are inhibitory cytokines, interferons, or TNF family members. In some embodiments the agents are combinations of the above classes of molecules. The agents can be derived from endogenous ligands or antibody molecules to the target receptors.

TABLE 2
T cell activation

| Activating Ligand | Target Receptor on T cell |
|---|---|
| B7-H2 (e.g., Accession Number | ICOS, CD28 (e.g., Accession |
| NP_056074.1) | Number NP_006130.1) |
| B7-1 (e.g., Accession Number | CD28 (e.g., Accession Number |
| NP_005182.1) | NP_006130.1) |
| B7-2 (e.g., Accession Number | CD28 (e.g., Accession Number |
| AAA86473) | NP_006130.1) |
| CD70 (e.g., Accession Number | CD27 (e.g., Accession Number |
| NP_001243.1) | NP_001233.1) |
| LIGHT (e.g., Accession Number | HVEM (e.g., Accession Number |
| NP_003798.2) | AAQ89238.1) |
| HVEM (e.g., Accession Number | LIGHT (e.g., Accession Number |
| AAQ89238.1) | NP_003798.2) |
| CD40L (e.g., Accession Number | CD40 (e.g., Accession Number |
| BAA06599.1) | NP_001241.1) |
| 4-1BBL (e.g., Accession Number | 4-1BB (e.g., Accession |
| NP_003802.1) | NP_001552.2) |
| OX40L (e.g., Accession Number | OX40 (e.g., Accession Number |
| NP_003317.1) | NP_003318.1) |
| TL1A (e.g., Accession Number | DR3 (e.g., Accession Number |
| NP_005109.2) | NP_683866.1) |
| GITRL (e.g., Accession Number | GITR (e.g., Accession Number |
| NP_005083.2) | NP_004186.1) |
| CD30L (e.g., Accession Number | CD30 (e.g., Accession Number |
| NP_001235.1), | NP_001234.3) |
| TIM4 (e.g., Accession Number | TIM1 (e.g., Accession Number |
| NP_612388.2) | NP_036338.2) |
| SLAM (e.g., Accession Number | SLAM (e.g., Accession Number |
| AAK77968.1) | AAK77968.1) |
| CD48 (e.g., Accession Number | CD2 (e.g., Accession Number |
| CAG33293.1) | NP_001315538.1) |
| CD58 (e.g., Accession Number | CD2 (e.g., Accession Number |
| CAG33220.1) | NP_001315538.1) |
| CD155 (e.g., Accession Number | CD226 (e.g., Accession Number |
| NP_001129240.1) | NP_006557.2) |
| CD112 (e.g., Accession Number | CD226 (e.g., Accession Number |
| NP_001036189.1) | NP_006557.2) |
| CD137L (e.g., Accession Number | CD137 (e.g., Accession |
| NP_003802.1) | NP_001552.2) |

In some embodiments, an anti-IL6 or TNFa antibody molecule comprises a sequence of either of SEQ ID NO: 48 or 49 herein, or a sequence with at least 70%, 80%, 85%, 90%, 95%, 96%, 97%, 98%, or 99% identity thereto.

Anti-IL6 scFv
SEQ ID NO: 48
EVQLVESGGGLVQPGGSLRLSCAASGFNFNDYFMNWVRQAPGKGLEWVAQM
RNKNYQYGTYYAESLEGRFTISRDDSKNSLYLQMNSLKTEDTAVYYCARES
YYGFTSYWGQGTLVTVSSGGGGSGGGGSGGGGSDIQMTQSPSSLSASVGDR
VTITCQASQDIGISLSWYQQKPGKAPKLLIYNANNLADGVPSRFSGSGSGT
DFTLTISSLQPEDFATYYCLQHNSAPYTFGQGTKLEIKR
Anti-TNFα scFv
SEQ ID NO: 49
EVQLVESGGGLVQPGRSLRLSCAASGFTFDDYAMHWVRQAPGKGLEWVSAI
TWNSGHIDYADSVEGRFTISRDNAKNSLYLQMNSLRAEDTAVYYCAKVSYL
STASSLDYWGQGTLVTVSSGGGGSGGGGSGGGGSDIQMTQSPSSLSASVGD
RVTITCRASQGIRNYLAWYQQKPGKAPKLLIYAASTLQSGVPSRFSGSGSG
TDFTLTISSLQPEDVATYYCQRYNRAPYTFGQGTKVEIK

In some embodiments, a T cell is inhibited using a ligand or receptor of Table 3, or a fragment or variant thereof. For instance, the first and second exogenous polypeptide can comprise a T cell inhibiting ligand (e.g., an inhibitory ligand of Table 3 or a fragment or variant thereof) and an agent that inhibits activation of a T cell (e.g., through a receptor of Table 2). In some embodiments, the agent that inhibits a T cell is an inhibitory ligand of Table 3, or a fragment or variant thereof. In some embodiments, the agent is an antibody molecule that binds a target receptor of Table 3, or a fragment or variant thereof.

TABLE 3
T cell inhibition

| Inhibitory Ligand | Target Receptor on T cell |
|---|---|
| B7-1 | CTLA4, B7H1 |
| B7-2 | CTLA4 |
| B7DC | PD1 |
| B7H1 | PD1, B7-1 |
| HVEM | CD160, BTLA |
| COLLAGEN | LAIR1 |
| GALECTIN9 | TIM3 |
| CD48, TIM4 | TIM4R |
| CD48 | 2B4 |
| CD155, CD112, CD113 | TIGIT |
| PDL1 | PD1 |

In embodiments, an engineered erythroid cell targets multiple T cell inhibitory pathways in combination (e.g., as described in Table 3), e.g., using ligands or antibody molecules, or both, co-expressed on an engineered erythroid cell. In some embodiments, the agents comprise a receptor or antibody molecule that captures inflammatory cytokines, e.g., to prevent additional activation of the target T cell. For example, the agents may comprise an agonist antibody molecule to the receptor CTLA-4 and an antibody molecule against TNFalpha.

In some embodiments the objective is to activate or to inhibit T cells. To ensure that T cells are preferentially targeted over other immune cells that may also express either activating or inhibitory receptors as described herein, one of the agents on the erythroid cell may comprise a targeting moiety, e.g., an antibody molecule that binds the T cell receptor (TCR) or another T cell marker. Targeting moieties are described in more detail in the section entitled “Localization configurations” herein. In some embodiments, a specific T cell subtype or clone may be enhanced or inhibited. In some embodiments, one or more of the agents on the erythroid cell is a peptide-MHC molecule that will selectively bind to a T cell receptor in an antigen-specific manner.

In some embodiments, an enucleated erythroid cell comprising a first exogenous polypeptide and a second exogenous polypeptide is administered to a subject having a first target and a second target. In embodiments, the first exogenous polypeptide acts on (e.g., binds) the first target and the second exogenous polypeptide acts on the second target. Optionally, the enucleated erythroid cell comprises a third exogenous polypeptide and the patient comprises a third target. In embodiments, the third exogenous polypeptide acts on the third target.

In some embodiments an erythroid cell comprises a first exogenous polypeptide which is an agonist or antagonist of a first target in a first pathway, and further comprises a second exogenous polypeptide which is an agonist or antagonist of a second target in a second pathway, wherein the first and second pathways act in concert toward a desired response. The first and second exogenous polypeptides can both be agonists; can both be antagonists; or one can be an agonist and the other can be an antagonist. In some embodiments, the target cell or tissue comprises inflamed tissue. In some embodiments, the erythroid cell further comprises a targeting agent.

Independent Function Configurations

When two or more agents (e.g., polypeptides) have an independent function relationship, the agents have two distinct (e.g., complementary) functions. For example, a first agent binds a first target and the second agent binds a second target. The patient may lack the first or second target. Optionally, the first and second agents are in different pathways.

In some embodiments, cellular damage is driven by a diverse mix of inflammatory cytokines. In an embodiment, the first and second peptides are molecules (e.g., antibody molecules) that bind two different cytokines. In some embodiments the agents bind and neutralize different cytokines and thus the engineered red cell product provides multifaceted protection from elevated cytokines.

In embodiments the cytokines comprise interleukins, e.g., IL-1, IL02, IL-3, IL-4, IL-5, IL-6, IL-7, IL-8, IL-9, IL-10, IL-11, IL-12, IL-13, IL-15, IL-16, IL-17, IL-18, IL-19, IL-20, IL-21, IL-22, IL-23, IL-25, IL-26, IL-27, IL-28, IL-29, IL-30, IL-31, IL-32, IL-33, IL-35, or IL-36. In some embodiments, the cytokine is a cytokine of Table 1 or a fragment or variant thereof. In some embodiments, the first cytokine is TNFa and the second is an interleukin, e.g., IL-6, or a fragment or variant of any of the foregoing. In some embodiments, the agents comprise anti-TNFa, anti-IL-6, or anti-IFNg antibody molecules, or any combination thereof, or a fragment or variant of any of the foregoing.

In some embodiments, an enucleated erythroid cell comprising a first exogenous polypeptide and a second exogenous polypeptide is administered to a subject having a first target but not a second target, or wherein the patient is not known to have a first target or second target. In embodiments, the first exogenous polypeptide acts on (e.g., binds) the first target and the second exogenous polypeptide remains substantially unbound. Optionally, the enucleated erythroid cell comprises a third exogenous polypeptide and the patient lacks a third target, or is not known to have the third target. In some embodiments, the enucleated erythroid cell comprises a plurality of exogenous polypeptides, and the patient does not have, or is not known to have, targets for one or a subset of the plurality of exogenous polypeptides.

Localization Configurations

When two or more agents (e.g., polypeptides) have a localization relationship, a first agent localizes the erythroid cell to a site of action that enhances the activity of the second or other agent or agents compared to their activity when not localized to the site of action (e.g., by binding of the first agent to its target, there is an increase in the local concentration of the second agent in the area of its target). In some embodiments one agent serves to target the erythroid cell to a site of action and one or more agents have a therapeutic effect. In an embodiment, binding of the first agent increases the activity of an entity, e.g., polypeptide, bound by the second agent. In an embodiment, the first agent binds to a substrate or product of the entity, e.g., polypeptide, bound by the second agent. The agent that localizes the erythroid cell may be, e.g., a ligand for a receptor on a target cell, or an antibody that binds a cell surface molecule on a target cell.

In some embodiments the site of action is a blood clot, the targeting agent binds the clot (e.g., fibrin), and the therapeutic agent is a fibrinolytic enzyme. The red cell therapeutic thus localizes to blood clots and breaks them open to prevent harmful emboli. In some embodiments, the targeting agent comprises an anti-fibrin antibody molecule, fibrin, or a fibrin-binding portion or variant thereof. In some embodiments, the fibrinolytic enzyme comprises plasmin or a fibrinolytic fragment or variant thereof.

As another example, an erythroid cell comprises a targeting agent that binds to inflamed vasculature and also comprises an anti-inflammatory molecule. In some embodiments, the targeting agent binds an inflammatory integrin, e.g., an avB3 integrin or an addressin such as MADCAM1. In some embodiments, the targeting agent comprises a lymphocyte homing receptor (e.g., CD34 or GLYCAM-1) or integrin-binding portion or variant thereof. In some embodiments, the anti-inflammatory molecule comprises an anti-inflammatory cytokine (e.g., IL-1 receptor antagonist, IL-4, IL-6, IL-10, IL-11, and IL-13), an inhibitor of TNF (e.g., an antibody molecule such as Enbrel), an inhibitor of a pro-inflammatory cytokine, an inhibitor against alpha4beta7 integrin (e.g., an antibody molecule), a colony-stimulating factor, a peptide growth factor, Monocyte Locomotion Inhibitory Factor (MLIF), Cortistatin, or an inhibitor of immune cell activation (e.g. PDL1 or another molecule described in Table 3 or antibody molecule thereto), or an anti-inflammatory variant (e.g., fragment) thereof.

In some embodiments, the erythroid cell targets a cell in an inflamed tissue. For instance, the cell can comprise one or more targeting agents, e.g., exogenous polypeptides that bind surface markers of inflamed tissue. The targeting agent can be an exogenous polypeptide comprising, e.g., an anti-VCAM antibody molecule or an anti-E-selectin antibody molecule. In embodiments, an erythroid cell comprises two targeting agents, which may increase the specificity and/or affinity and/or avidity of the erythroid cell binding to its target, compared to an otherwise similar erythroid cell comprising only one of the targeting agents. In embodiments, the targeting moieties comprise: a surface exposed anti-VCAM antibody molecule and a surface exposed anti-E-selectin antibody molecule; a surface exposed alpha4Beta1 integrin or fragment or variant thereof and a surface exposed anti-E-selectin antibody molecule; or a surface exposed alphavbeta2 integrin or fragment or variant thereof and a surface exposed anti-E-selectin antibody molecule. The erythroid cell optionally further comprises an exogenous polypeptide with therapeutic activity, e.g., anti-inflammatory activity. The exogenous polypeptide with therapeutic activity can comprise an enzyme, capture reagent, agonist, or antagonist.

In embodiments, the targeting moiety comprises a receptor or a fragment or variant thereof. In embodiments, the targeting moiety comprises an antibody molecule such as an scFv.

In embodiments, the first exogenous polypeptide can comprise a targeting agent and the second exogenous polypeptide can comprise an enzyme. For example, in some embodiments, the erythroid cell comprises a first polypeptide comprising a targeting agent that binds a cell and a second polypeptide that inhibits the cell. For instance, the targeting agent can comprise an anti-CD4 antibody which binds CD4 on the surface of a T cell. The second polypeptide can comprise an enzyme which can be surface-exposed or intracellular, e.g., intracellular and not membrane associated. The enzyme may be IDO or a fragment or variant thereof, which depletes tryptophan and can induce anergy in the cell, or ADA or a fragment or variant thereof. The enzyme may be a protease. In embodiments, the first polypeptide comprises an anti-MAdCAM-1 antibody molecule and the second polypeptide comprises LysC or LysN (which can cleave MAdCAM-1 at 1 or more (e.g., 2, 3, or 4 sites), e.g., wherein the target tissue is inflamed tissue.

The first exogenous polypeptide can comprise a targeting agent and the second exogenous polypeptide can comprise an agonist of a target. For instance, in some embodiments, the first exogenous polypeptide comprises an anti-MAdCAM-1 antibody molecule, e.g., which can bind MAdCAM-1, e.g., on inflamed tissue. The second exogenous polypeptide may comprise an anti-inflammatory molecule, e.g., IL10 or a fragment or variant thereof. In embodiments, the targeting agent comprises a receptor or fragment or variant thereof, an antibody molecule, a ligand or fragment or variant thereof, a cytokine or fragment or variant thereof. In embodiments, the second exogenous polypeptide comprises an attenuator, an activator, a cell-killing agent, or a cytotoxic molecule (e.g., a small molecule, protein, RNA e.g., antisense RNA, or TLR ligand). In embodiments, the second exogenous polypeptide is intracellular, e.g., not membrane associated, and in some embodiments, the second exogenous polypeptide is surface-exposed.

The erythroid cell can comprise a targeting agent and a capture agent. For example, the first exogenous polypeptide can comprise a targeting agent that binds a cell, e.g., a plasma cell, e.g., an anti-BCMA antibody molecule. The second exogenous polypeptide may capture cytokines, e.g., may comprise TACI or a fragment or variant thereof which can capture BLyS (also called BAFF) and/or APRIL. In embodiments, the second exogenous polypeptide binds its target in a way that prevents the target from interacting with an endogenous receptor, e.g., binds the target at a moiety that overlaps with the receptor binding site. In embodiments, the targeting moiety binds a receptor at the site of disease, e.g., binds an integrin. In embodiments, the target cell is inflamed tissue. In embodiments, the targeting agent comprises a ligand or a cytokine or fragment or variant thereof, or an antibody molecule, e.g., an scFv. In embodiments, the capture agent comprises a receptor or fragment or variant thereof, or an antibody molecule, e.g., an scFv. In embodiments, the ligand is an unwanted cytokine or chemokine.

Proximity-Based Configurations

When two or more agents (e.g., polypeptides) have a proximity-based relationship, the two agents function more strongly, e.g., exert a more pronounced effect, when they are in proximity to each other than when they are physically separate. In embodiments, the two agents are in proximity when they are directly binding to each other, when they are part of a complex (e.g., linked by a third agent), when they are present on the same cell membrane, or when they are present on the same subsection of a cell membrane (e.g., within a lipid raft, outside a lipid raft, or bound directly or indirectly to an intracellular structure such as a cytoskeleton component). In some embodiments, first polypeptide binds a first target molecule and the second polypeptide binds a second target molecule, and this binding causes the first target molecule and the second target molecule to move into closer proximity with each other, e.g., to bind each other. In some embodiments, the first and second target molecules are cell surface receptors on a target cells.

In an embodiment, an erythroid cell comprises an optional first exogenous polypeptide, a second exogenous polypeptide, and a third exogenous polypeptide. The second and third exogenous polypeptides can bind to different epitopes within the same polypeptide chain of a target, e.g., cytokine B. The second and third exogenous polypeptides, which are mounted on the erythrocyte, bind to the target with higher avidity than if the second and third exogenous polypeptides were free polypeptides. As examples, two or more exogenous polypeptides could bind different sites on the same target, wherein the target is a cytokine, a complement factor (e.g., C5), an enzyme, an antibody, or an immune complex. As an example, the first exogenous polypeptide comprises a targeting moeity.

Scaffold Configurations

When two or more agents (e.g., polypeptides) have a scaffold relationship, the agents bring two or more targets together, to increase the likelihood of the targets interacting with each other. In an embodiment the first and second agent are associated with each other (forming a scaffold) at the surface of the erythroid cell, e.g., two complexed polypeptides. In an embodiment, the erythroid cell comprises a bispecific antibody molecule, e.g., an antibody molecule that recognizes one or more (e.g., 2) proteins described herein, e.g., in any of Table 1, Table 2, Table 3, and Table 4.

The targets may comprise, e.g., proteins, cells, small molecules, or any combination thereof. In an embodiment, the first and second targets are proteins. In an embodiment, the first and second targets are cells.

As an example, an agent (e.g., an antibody molecule) that binds Factor IX and an agent (e.g., an antibody molecule) that binds Factor X brings these two clotting factors together, a role typically played by Factor VIII. In embodiments, this serves as a treatment for Factor VIII-deficient hemophilia (Hemophilia A).

In some embodiments, an erythroid cell expresses an exogenous fusion polypeptide comprising a first antibody molecule domain and a second antibody molecule domain, wherein the exogenous polypeptide functions as a bispecific antibody, e.g., wherein the first antibody molecule domain binds a first target on a first cell and the second antibody molecule domain binds a second target on a second cell, e.g., a different cell type.

Multimer Configurations

When two or more agents (e.g., polypeptides) have a multimer configuration, the agents combine with each other, e.g., bind each other, to form a complex that has a function or activity on a target.

In some embodiments, an enucleated erythroid cell acts on a complement cascade. Some complement regulatory proteins act in concert as co-factors for one another, e.g. CFH and CD55 are co-factors for the enzymatic activity of CFI. In some embodiments, the agents comprise an enzymatic protein or domain, e.g., CFI, and a co-factor, e.g., CFH or CD55, that accelerates and enhances the activity of CFI on the target complement molecule.

In some embodiments the complex comprises multiple subdomains derived from different polypeptide chains, all of which must be expressed in order for the complex to be active.

In some embodiments, the complex comprises a plurality of clotting factors.

Pathway Biology Configurations

When two or more agents (e.g., polypeptides) have a pathway biology relationship, the agents act on successive steps of a pathway, e.g., to modulate complex systems. As an example, the first exogenous polypeptide is an enzyme that converts a substrate into an intermediate, and the second exogenous polypeptide is an enzyme that converts the intermediate into a product. Co-localization of the enzymes on or in the same erythroid cell allow the reaction to take place faster than it would in solution. In some embodiments, one, two, or more, of the enzymes are not natively found in humans; this would enable new metabolic pathways to be introduced.

In some embodiments, the enzymes synthesize a beneficial product. In some embodiments, the enzymes break down a harmful substrate.

In some embodiments, e.g., for treating PKU, an agent comprises phenylalanine ammonia lyase (PAL) or a fragment or variant thereof. In some embodiments, e.g., for treating ADA-SCID, an agent comprises adenosine deaminase (ADA) or a fragment or variant thereof. In some embodiments, e.g., for treating Mitochondrial Neurogastrointestinal Encephalopathy, an agent comprises thymidine phosphorylase or a fragment or variant thereof. In some embodiments, e.g., for treating Primary Hyperoxaluria, an agent comprises oxalate oxidase or a fragment or variant thereof. In some embodiments, e.g., for treating Alkaptonuria, an agent comprises homogentisate oxidase or a fragment or variant thereof. In some embodiments, e.g., for treating Thrombotic Thrombocytopenic Purpura, an agent comprises ADAMTS13 or a fragment or variant thereof.

In some embodiments, the first exogenous polypeptide produces a reaction product, e.g., an undesired reaction product, e.g., peroxide, and the second exogenous polypeptide breaks down the reaction product. For example in embodiments, the first exogenous polypeptide comprises uricase, which acts on uric acid and produces peroxide, and the second exogenous polypeptide comprises catalase, which breaks down the peroxide.

In some embodiments, the first exogenous polypeptide has a first K_{M} for a first substrate and the second exogenous polypeptide has a second K_{M} for a second substrate. In embodiments, the first K_{M} is less than 90%, 80%, 70%, 60%, 50%, 40%, 30%, 20%, or 10% of the second K_{M}. In embodiments, the first K_{M} is at least 90%, 80%, 70%, 60%, 50%, 40%, 30%, 20%, or 10% of the second K_{M}.

Compensatory Configurations

When two or more agents (e.g., polypeptides) have a compensatory relationship, a first agent reduces an undesirable characteristic of a second agent. For example, in some embodiments, the second agent has a given level of immunogenicity, and the first agent reduces the immunogenicity, e.g., by negatively signaling immune cells (see Table 3), or by shielding an antigenic epitope of the second agent. In some embodiments, the second agent has a given half-life, and the first agent increases the half-life of the second agent. For example, the first agent can comprise a chaperone or fragment or variant thereof.

An enucleated erythroid cell can co-express a therapeutic protein and its inhibitor. The inhibitor can be released (e.g., cease binding the therapeutic but remain on the surface of the cell) at the desired location in the body, to activate the therapeutic protein.

For instance, in some embodiments, the erythroid cell comprises a first exogenous polypeptide with therapeutic activity (e.g., an anti-TNFalpha antibody molecule), a second exogenous polypeptide (e.g., TNFalpha or a fragment or variant thereof) that inhibits the first exogenous polypeptide. The second polypeptide (e.g., TNFalpha) may inhibit activity of the first exogenous polypeptide (e.g., anti-TNFalpha) until the erythroid cell is at a desired location, e.g., at inflamed tissue, e.g., limiting off-target effects. The second exogenous polypeptide (e.g., TNFalpha) may comprise a variant of the target (e.g., endogenous TNFalpha) that the first exogenous polypeptide (e.g., anti-TNFalpha) binds. For instance, the variant can be a weakly-binding variant that is competed away in the presence of the target. In embodiments, the Kd of the first exogenous polypeptide for the second exogenous polypeptide is at least 2, 3, 5, 10, 20, 50, or 100-fold greater than the Kd of the first exogenous polypeptide for its target. The erythroid cell optionally comprises a third exogenous polypeptide that comprises a targeting agent.

In some embodiments, the enucleated erythroid cell comprises a prodrug (e.g., pro-insulin) that becomes a drug (e.g., insulin) at a desired site in a subject.

Enucleated Erythroid Cells Comprising Three or More Agents (e.g., Polypeptides)

In embodiments, an enucleated erythroid cell described herein comprises three or more, e.g., at least 4, 5, 10, 20, 50, 100, 200, 500, or 1000 agents. In embodiments, a population of erythroid cells described herein comprises three or more, e.g., at least 4, 5, 10, 20, 50, 100, 200, 500, 1000, 2000, or 5000 agents, e.g., wherein different erythroid cells in the population comprise different agents or wherein different erythroid cells in the population comprise different pluralities of agents. In embodiments, two or more (e.g., all) of the agents in the erythroid cell or population of erythroid cell have agent-additive, agent-synergistic, multiplicative, independent function, localization-based, proximity-dependent, scaffold-based, multimer-based, pathway-based, or compensatory activity.

In embodiments, the erythroid cell is produced by contacting an erythroid cell progenitor cell with a plurality of mRNAs encoding the agents.

### Physical Characteristics of Enucleated Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an enucleated erythroid cell that expresses an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the enucleated erythroid cell comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the enucleated erythroid cell exhibits substantially the same osmotic membrane fragility as an isolated, uncultured erythroid cell that does not comprise an exogenous polypeptide. In some embodiments, the population of enucleated erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the enucleated erythroid cell has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the enucleated erythroid cell has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the enucleated erythroid cell has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587, which is herein incorporated by reference in its entirety.

Other Characteristics

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the erythroid cells have a half-life of at least 0.5, 1, 2, 7, 14, 30, 45, or 90 days in a subject.

In some embodiments, a population of cells comprising erythroid cells comprises less than about 10, 5, 4, 3, 2, or 1% echinocytes.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell, e.g., an erythroid cell, contains a nucleus that is non-functional, e.g., has been inactivated.

### Methods of Manufacturing Enucleated Erythroid Cells

Methods of manufacturing enucleated erythroid cells (e.g., reticulocytes) comprising (e.g., expressing) exogenous agent (e.g., polypeptides) are described, e.g., in WO2015/073587 and WO2015/153102, each of which is incorporated by reference in its entirety.

In some embodiments, hematopoietic progenitor cells, e.g., CD34+hematopoietic progenitor cells, are contacted with a nucleic acid or nucleic acids encoding one or more exogenous polypeptides, and the cells are allowed to expand and differentiate in culture.

In some embodiments, the two or more polypeptides are encoded in a single nucleic acid, e.g. a single vector. In embodiments, the single vector has a separate promoter for each gene, has two proteins that are initially transcribed into a single polypeptide having a protease cleavage site in the middle, so that subsequent proteolytic processing yields two proteins, or any other suitable configuration. In some embodiments, the two or more polypeptides are encoded in two or more nucleic acids, e.g., each vector encodes one of the polypeptides.

The nucleic acid may be, e.g., DNA or RNA. A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, the cells are produced using sortagging, e.g., as described in WO02014/183071 or WO2014/183066, each of which is incorporated by reference in its entirety.

Erythroid cells described herein can also be produced using coupling reagents to link an agent (e.g., an exogenous polypeptide) to a cell. For instance, click chemistry can be used. Coupling reagents can be used to couple an agent to a cell, for example, when the agent is a complex or difficult to express agent, e.g., a polypeptide, e.g., a multimeric polypeptide; large polypeptide; agent derivatized in vitro, e.g., polypeptide; agent that may have toxicity to, or which are not expressed efficiently in, the erythroid cells.

Thus, in some embodiments, an erythroid cell described herein comprises many as, at least, more than, or about 5,000, 10,000, 50,000, 100,000, 200,000, 300,000, 400,000, 500,000 coupling reagents per cell. In some embodiments, the erythroid cells are made by a method comprising a) coupling a first coupling reagent to an erythroid cell, thereby making a pharmaceutical preparation, product, or intermediate. In an embodiment, the method further comprises: b) contacting the cell with an agent coupled to a second coupling reagent e.g., under conditions suitable for reaction of the first coupling reagent with the second coupling reagent. In embodiments, two or more agents are coupled to the cell (e.g., using click chemistry). In embodiments, a first agent is coupled to the cell (e.g., using click chemistry) and a second agent comprises a polypeptide expressed from an exogenous nucleic acid.

In some embodiments, the coupling reagent comprises an azide coupling reagent. In some embodiments, the azide coupling reagent comprises an azidoalkyl moiety, azidoaryl moiety, or an azidoheteroaryl moiety. Exemplary azide coupling reagents include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. Coupling reagents may also comprise an alkene moiety, e.g., a transcycloalkene moiety, an oxanorbornadiene moiety, or a tetrazine moiety. Additional coupling reagents can be found in Click Chemistry Tools (https://clickchemistrytools.com/) or Lahann, J (ed) (2009) Click Chemistry for Biotechnology and Materials Science, each of which is incorporated herein by reference in its entirety.

In some embodiments, the erythroid cells are expanded at least 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, the population of erythroid cells comprises at least 30, 35, 40, 45, 50, 55, 60, 65, 70, 75, 80, 85, 90, 95, or 98% (and optionally up to about 80, 90, or 100%) enucleated erythroid cells. In some embodiments, the population of erythroid cells contains less than 1% live enucleated cells, e.g., contains no detectable live enucleated cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least 30, 35, 40, 45, 50, 55, 60, 65, 70, 75, or 80% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

### Physically Proximal, Synergistic Agents

In some aspects, the present disclosure provides a composition comprising a first agent and a second agent in physical proximity to each other. In some embodiments, agents act synergistically when they are in physical proximity to each other but not when they are separate. In some embodiments, the first and second agents are covalently linked, e.g., are part of a fusion protein or are chemically conjugated together. In some embodiments, the first and second agent are non-covalently linked, e.g., are bound directly to each other or to a scaffold. In some embodiments, the first and second agents are part of (e.g., linked to) a nanoparticle (e.g., 1-100, 100-2,500, or 2,500-10,000 nm in diameter) liposome, vesicle, bead, polymer, implant, or polypeptide complex.

In some embodiments, the composition comprises at least 3, 4, 5, 6, 7, 8, 9, or 10 different agents that are in physical proximity to each other (e.g., covalently or noncovalently linked).

In some embodiments, the composition comprises one or more (e.g., 2, 3, 4, 5, or more) agents described herein, e.g., exogenous polypeptides described herein, e.g., polypeptides of any of Table 1, Table 2, Table 3, or Table 4, or a fragment or variant thereof, or an antibody molecule thereto. In some embodiments, one or more (e.g., 2, 3, or more) of the exogenous polypeptides comprise a first polypeptide of Table 4 and a second polypeptide of Table 4.

### Engineered Erythroid Cells Comprising One or More Agents

In some aspects, the present disclosure provides an engineered erythroid cell comprising an exogenous agent. More specifically, in some aspects, the present disclosure provides an enucleated erythroid cell comprising an exogenous polypeptide. The erythroid cell optionally further comprises a second, different, exogenous polypeptide.

In some embodiments, the exogenous polypeptide (e.g., an exogenous polypeptide comprised by an enucleated erythroid cell that optionally further comprises a second exogenous polypeptide) is an exogenous polypeptide described herein. In embodiments, the polypeptide is selected from any of Table 1, Table 2, Table 3, or Table 4, or a fragment or variant thereof, or an antibody molecule thereto.

In some embodiments, the exogenous polypeptide comprises a cardiovascular therapeutic, e.g., an anti-PCSK9 antibody or Ecallantaide or a combination thereof, e.g., for the treatment of a cardiovascular disease.

### Vehicles for Polypeptides Described Herein

While in many embodiments herein, the one or more (e.g., two or more) exogenous polypeptides are situated on or in an enucleated erythroid cell, it is understood that any exogenous polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more agents described herein. In some embodiments, the one or more agents comprise an agent selected a polypeptide of any of Table 1, Table 2, Table 3, or Table 4, or a fragment or variant thereof, or an agonist or antagonist thereof, or an antibody molecule thereto. In some embodiments, the vehicle comprises two or more agents described herein, e.g., any pair of agents described herein.

In some embodiments, the vehicle comprises an erythroid cell. In embodiments, the erythroid cell is a nucleated red blood cell, red blood cell precursor, or enucleated red blood cell. In embodiments, the erythroid cell is a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, or a combination thereof. In some embodiments, the erythroid cells are immortal or immortalized cells.

### Heterogeneous Populations of Cells

While in many embodiments herein, the one or more (e.g., two or more) exogenous polypeptides are situated on or in a single cell, it is understood that any exogenous polypeptide or combination of exogenous polypeptides described herein can also be situated on a plurality of cells. For instance, in some aspects, the disclosure provides a plurality of erythroid cells, wherein a first cell of the plurality comprises a first agent (e.g., an exogenous polypeptide, e.g., an exogenous polypeptide described herein) and a second cell of the plurality comprises a second agent (e.g., an exogenous polypeptide, e.g., an exogenous polypeptide described herein). In some embodiments, the plurality of cells comprises two or more agents described herein, e.g., any pair of agents described herein. In some embodiments, less than 90%, 80%, 70%, 60%, 50%, 40%, 30%, 20%, 10%, 5%, 2%, or 1% of the cells in the population comprise both the first exogenous polypeptide and the second exogenous polypeptide.

### Cells Encapsulated in a Membrane

In some embodiments, enucleated erythroid cells or other vehicles described herein are encapsulated in a membrane, e.g., semi-permeable membrane. In embodiments, the membrane comprises a polysaccharide, e.g., an anionic polysaccharide alginate. In embodiments, the semipermeable membrane does not allow cells to pass through, but allows passage of small molecules or macromolecules, e.g., metabolites, proteins, or DNA. In embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

Methods of Treatment with Compositions Herein, e.g., Enucleated Erythroid Cells

Methods of administering enucleated erythroid cells (e.g., reticulocytes) comprising (e.g., expressing) exogenous agent (e.g., polypeptides) are described, e.g., in WO2015/073587 and WO2015/153102, each of which is incorporated by reference in its entirety.

In embodiments, the enucleated erythroid cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications.

In some embodiments, the erythroid cells are administered to a patient every 1, 2, 3, 4, 5, or 6 months.

In some embodiments, a dose of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, the erythroid cells are administered to a patient in a dosing regimen (dose and periodicity of administration) sufficient to maintain function of the administered erythroid cells in the bloodstream of the patient over a period of 2 weeks to a year, e.g., one month to one year or longer, e.g., at least 2 weeks, 4 weeks, 6 weeks, 8 weeks, 3 months, 6 months, a year, 2 years.

In some aspects, the present disclosure provides a method of treating a disease or condition described herein, comprising administering to a subject in need thereof a composition described herein, e.g., an enucleated erythroid cell described herein. In some embodiments, the disease or condition is cardiovascular disease or a metabolic disease (e.g., metabolic deficiency). In some aspects, the disclosure provides a use of an erythroid cell described herein for treating a disease or condition described herein, e.g., cardiovascular disease or a metabolic disease. In some aspects, the disclosure provides a use of an erythroid cell described herein for manufacture of a medicament for treating a disease or condition described herein, e.g., a cardiovascular disease or a metabolic disease.

In some embodiments, the cardiovascular disease is heart failure, pulmonary hypertension, high blood pressure, stroke (e.g., embolic stroke, thrombotic stroke, or hemorrhagic stroke), embolism, or occlusionary or hemorrhagic lesion or incident, e.g., stroke or embolism. In some embodiments, the cardiovascular disease is characterized by excessive blood clotting, e.g., pulmonary embolism, myocardial infarction or stroke.

In some embodiments, an erythroid cell described herein is administered (e.g., to a patient having a cardiovascular disease) in combination with one or more second therapies. The second therapy may be, e.g., surgery or a drug. Exemplary second therapies include use of a stent, catheter, angioplasty, valve, or pacemaker. Exemplary second therapies also include valve repair or bypass surgery.

In embodiments, the metabolic disease is osteoporosis, diabetes (e.g., type 1, type 2, or gestational diabetes), insulin resistance, or obesity.

Metabolic deficiencies include Phenylketonuria (PKU), Adenosine Deaminase Deficiency-Severe Combined Immunodeficiency (ADA-SCID), Mitochondrial Neurogastrointestinal Encephalopathy (MNGIE), Primary Hyperoxaluria, Alkaptonuria, Thrombotic Thrombocytopenic Purpura (TTP), homocystinuria, and hyperuricemia.

An enucleated erythroid cell population may comprise, for example, a plurality (e.g., 2, 3, 4, or 5 or more) of cardiovascular therapeutic polypeptides or metabolic therapeutic polypeptides. The enucleated erythroid cell populations comprising cardiovascular therapeutic polypeptides may be formulated in a pharmaceutical composition comprising an appropriate excipient (e.g., AS-3 additive solution) and administered, e.g., intravenously, to a patient, e.g., a patient suffering from a cardiovascular disease (e.g. heart failure, atherosclerosis, thromboembolism, hypercholesterolemia, or hereditary angioedema). The enucleated erythroid cell populations comprising metabolic therapeutic polypeptides may be formulated in a pharmaceutical composition comprising an appropriate excipient (e.g., AS-3 additive solution) and administered, e.g., intravenously, to a patient, e.g., a patient suffering from a metabolic disorder (e.g., diabetes (e.g., type 1 diabetes, type 2 diabetes, or gestational diabetes), insulin insensitivity, and obesity) such as a metabolic deficiency (e.g., hemophilia (e.g., hemophilia type A, hemophilia type B, or hemophilia type C), von Willebrand disease, Factor II deficiency, Factor V deficiency, Factor VII deficiency, Factor X deficiency, Factor XII deficiency, thrombotic thrombocytopenic purpura, Phenylketonuria (PKU), Adenosine Deaminase Deficiency-Severe Combined Immunodeficiency (ADA-SCID), Mitochondrial Neurogastrointestinal Encephalopathy (MNGIE), Primary Hyperoxaluria, Alkaptonuria, Thrombotic Thrombocytopenic Purpura (TTP)), homocystinuria, and hyperuricemia.

### ADDITIONAL TABLES

TABLE 4
Amino acid sequences of cardiovascular therapeutic polypeptides
and metabolic therapeutic polypeptides described herein.

| SEQ ID | Sequence | | |
|---|---|---|---|
| NO: | Name | ProtID | Amino acid sequence |
| 1 | Human | P01275 | HAEGTFTSDVSSYLEGQAAKEFIAWLVKGRG |
| | GLP-1 | (98-128) | |
| | (hGLP-1) | | |
| 2 | Human | P01308 | Insulin A chain 90-110 |
| | insulin | | GIVEQCCTSICSLYQLENYCN |
| | | | Insulin B chain 25-54 |
| | | | FVNQHLCGSHLVEALYLVCGERGFFYTPKT |
| 3 | Linker | | GGGSGGGSGGGS |
| 4 | HA peptide | | YPYDVPDY |
| 5 | FGF-21 | Uniprot | HPIPDSSPLLQFGGQVRQRYLYTDDAQQTEAHLEIREDG |
| | | Q9NSAl | TVGGAADQSPESLLQLKALKPGVIQILGVKTSRFLCQRP |
| | | | DGALYGSLHFDPEACSFRELLLEDGYNVYQSEAHGLPL |
| | | | HLPGNKSPHRDPAPRGPARFLPLPGLPPALPEPPGILAPQ |
| | | | PPDVGSSDPLSMVGPSQGRSPSYAS |
| 6 | Glucagon | Uniprot | RSLQDTEEKSRSFSASQADPLSDPDQMNEDKRHSQGTF |
| | | P01275 | TSDYSKYLDSRRAQDFVQWLMNTKRNRNNIAKRHDEF |
| | | | ERHAEGTFTSDVSSYLEGQAAKEFIAWLVKGRGRRDFP |
| | | | EEVAIVEELGRRHADGSFSDEMNTILDNLAARDFINWLI |
| | | | QTKITDRK |
| 7 | Leptin | Uniprot | VPIQKVQDDTKTLIKTIVTRINDISHTQSVSSKQKVTGLD |
| | | P41159 | FIPGLHPILTLSKMDQTLAVYQQILTSMPSRNVIQISNDL |
| | | | ENLRDLLHVLAFSKSCHLPWASGLETLDSLGGVLEASG |
| | | | YSTEVVALSRLQGSLQDMLWQLDLSPGC |
| 8 | Human | Uniprot | SPKMVQGSGCFGRKMDRISSSSGLGCKVLRRH |
| | brain | P16860 | |
| | natriuretic | (103-134) | |
| | peptide | | |
| | (hBNP) | | |
| 9, | Human | P04090 | Relaxin A chain |
| 31 | relaxin | (25-53) | DSWMEEVIKLCGRELVRAQIAICGMSTWS (SEQ ID NO: |
| | | P04090 | 9) |
| | | (162-185) | Relaxin B chain |
| | | | QLYSALANKCCHVGCTKRSLARFC (SEQ ID NO: 31) |
| 10 | tPA v1 | Uniprot | SYQVICRDEKTQMIYQQHQSWLRPVLRSNRVEYCWCN |
| | | P00750 | SGRAQCHSVPVKSCSEPRCFNGGTCQQALYFSDFVCQC |
| | | | PEGFAGKCCEIDTRATCYEDQGISYRGTWSTAESGAECT |
| | | | NWNSSALAQKPYSGRRPDAIRLGLGNHNYCRNPDRDS |
| | | | KPWCYVFKAGKYSSEFCSTPACSEGNSDCYFGNGSAYR |
| | | | GTHSLTESGASCLPWNSMILIGKVYTAQNPSAQALGLG |
| | | | KHNYCRNPDGDAKPWCHVLKNRRLTWEYCDVPSCSTC |
| | | | GLRQYSQPQFRIKGGLFADIASHPWQAAIFAKHRRSPGE |
| | | | RFLCGGILISSCWILSAAHCFQERFPPHHLTVILGRTYRV |
| | | | VPGEEEQKFEVEKYIVHKEFDDDTYDNDIALLQLKSDSS |
| | | | RCAQESSVVRTVCLPPADLQLPDWTECELSGYGKHEAL |
| | | | SPFYSERLKEAHVRLYPSSRCTSQHLLNRTVTDNMLCA |
| | | | GDTRSGGPQANLHDACQGDSGGPLVCLNDGRMTLVGII |
| | | | SWGLGCGQKDVPGVYTKVTNYLDWIRDNMRP |
| 11 | tPA v2 | Drugbank | SYQVICRDEKTQMIYQQHQSWLRPVLRSNRVEYCWCN |
| | | DB00009 | SGRAQCHSVPVKSCSEPRCFNGGTCQQALYFSDFVCQC |
| | | | PEGFAGKCCEIDTRATCYEDQGISYRGTWSTAESGAECT |
| | | | NWNSSALAQKPYSGRRPDAIRLGLGNHNYCRNPDRDS |
| | | | KPWCYVFKAGKYSSEFCSTPACSEGNSDCYFGNGSAYR |
| | | | GTHSLTESGASCLPWNSMILIGKVYTAQNPSAQALGLG |
| | | | KHNYCRNPDGDAKPWCHVLKNRRLTWEYCDVPSCSTC |
| | | | GLRQYSQPQFRIKGGLFADIASHPWQAAIFAKHRRSPGE |
| | | | RFLCGGILISSCWILSAAHCFQERFPPHHLTVILGRTYRV |
| | | | VPGEEEQKFEVEKYIVHKEFDDDTYDNDIALLQLKSDSS |
| | | | RCAQESSVVRTVCLPPADLQLPDWTECELSGYGKHEAL |
| | | | SPFYSERLKEAHVRLYPSSRCTSQHLLNRTVTDNMLCA |
| | | | GDTRSGGPQANLHDACQGDSGGPLVCLNDGRMTLVGII |
| | | | SWGLGCGQKDVPGVYTKVTNYLDWIRDNMRP |
| 12 | tPA v3 | Drugbank | SYQGNSDCYFGNGSAYRGTHSLTESGASCLPWNSMILI |
| | | DB00015 | GKVYTAQNPSAQALGLGKHNYCRNPDGDAKPWCHVL |
| | | | KNRRLTWEYCDVPSCSTCGLRQYSQPQFRIKGGLFADI |
| | | | ASHPWQAAIFAKHRRSPGERFLCGGILISSCWILSAAHCF |
| | | | QERFPPHHLTVILGRTYRVVPGEEEQKFEVEKYIVHKEF |
| | | | DDDTYDNDIALLQLKSDSSRCAQESSVVRTVCLPPADL |
| | | | QLPDWTECELSGYGKHEALSPFYSERLKEAHVRLYPSS |
| | | | RCTSQHLLNRTVTDNMLCAGDTRSGGPQANLHDACQG |
| | | | DSGGPLVCLNDGRMTLVGIISWGLGCGQKDVPGVYTK |
| | | | VTNYLDWIRDNMRP |
| 13 | tPA v4 | Drugbank | SYQVICRDEKTQMIYQQHQSWLRPVLRSNRVEYCWCN |
| | | DB00031 | SGRAQCHSVPVKSCSEPRCFNGGTCQQALYFSDFVCQC |
| | | | PEGFAGKCCEIDTRATCYEDQGISYRGNWSTAESGAEC |
| | | | TQWNSSALAQKPYSGRRPDAIRLGLGNHNYCRNPDRDS |
| | | | KPWCYVFKAGKYSSEFCSTPACSEGNSDCYFGNGSAYR |
| | | | GTHSLTESGASCLPWNSMILIGKVYTAQNPSAQALGLG |
| | | | KHNYCRNPDGDAKPWCHVLKNRRLTWEYCDVPSCSTC |
| | | | GLRQYSQPQFRIKGGLFADIASHPWQAAAAAKHRRSPG |
| | | | ERFLCGGILISSCWILSAAHCFQERFPPHHLTVILGRTYR |
| | | | VVPGEEEQKFEVEKYIVHKEFDDDTYDNDIALLQLKSD |
| | | | SSRCAQESSVVRTVCLPPADLQLPDWTECELSGYGKHE |
| | | | ALSPFYSERLKEAHVRLYPSSRCTSQHLLNRTVTDNML |
| | | | CAGDTRSGGPQANLHDACQGDSGGPLVCLNDGRMTLV |
| | | | GIISWGLGCGQKDVPGVYTKVTNYLDWIRDNMRP |
| 14 | TFPI | uniprot | DSEEDEEHTIITDTELPPLKLMHSFCAFKADDGPCKAIM |
| | | P10646 | KRFFFNIFTRQCEEFIYGGCEGNQNRFESLEECKKMCTR |
| | | | DNANRIIKTTLQQEKPDFCFLEEDPGICRGYITRYFYNNQ |
| | | | TKQCERFKYGGCLGNMNNFETLEECKNICEDGPNGFQV |
| | | | DNYGTQLNAVNNSLTPQSTKVPSLFEFHGPSWCLTPAD |
| | | | RGLCRANENRFYYNSVIGKCRPFKYSGCGGNENNFTSK |
| | | | QECLRACKKGFIQRISKGGLIKTKRKRKKQRVKIAYEEI |
| | | | FVKNM |
| 15 | Anti- | Drug | EVQLLESGGGLVQPGGSLRLSCAASGFTFSSYAMSWVR |
| | huRANK- | bank | QAPGKGLEWVSGITGSGGSTYYADSVKGRFTISRDNSK |
| | L (heavy | number | NTLYLQMNSLRAEDTAVYYCAKDPGTTVIMSWFDPWG |
| | chain | DB06643 | QGTLVTVSSASTKGPSVFPLAPSSKSTSGGTAALGCLVK |
| | sequence) | | DYFPEPVTVSWNSGALTSGVHTFPAVLQSSGLYSLSSVV |
| | | | TVPSSSLGTQTYICNVNHKPSNTKVDKKVEPKSCDKTH |
| | | | TCPPCPAPELLGGPSVFLFPPKPKDTLMISRTPEVTCVVV |
| | | | DVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSRDELTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| 16 | Anti- | Drug | EIVLTQSPGTLSLSPGERATLSCRASQSVRGRYLAWYQQ |
| | huRANK- | bank | KPGQAPRLLIYGASSRATGIPDRFSGSGSGTDFTLTISRL |
| | L (light | number | EPEDFAVFYCQQYGSSPRTFGQGTKVEIKRTVAAPSVFI |
| | chain | DB06643 | FPPSDEQLKSGTASVVCLLNNFYPREAKVQWKVDNAL |
| | sequence) | | QSGNSQESVTEQDSKDSTYSLSSTLTLSKADYEKHKVY |
| | | | ACEVTHQGLSSPVTKSFNRGEC |
| 17 | Human | P01270 | SVSEIQLMHNLGKHLNSMERVEWLRKKLQDVHNF |
| | parathyroid | (PTHY_HUMAN) | |
| | hormone) | | |
| | (1-34) | | |
| 18 | Anabaena | Uniprot | MKTLSQAQSK TSSQQFSFTG NSSANVIIGN |
| | variabilis | Q3M5Z3 | QKLTINDVAR VARNGTLVSL TNNTDILQGI |
| | phenylalanine | | QASCDYINNA VESGEPIYGV TSGFGGMANV |
| | ammonia | | AISREQASEL QTNLVWFLKT GAGNKLPLAD |
| | lyase | | VRAAMLLRAN SHMRGASGIR LELIKRMEIF |
| | | | LNAGVTPYVY EFGSIGASGD LVPLSYITGS |
| | | | LIGLDPSFKV DFNGKEMDAP |
| | | | TALRQLNLSP LTLLPKEGLA MMNGTSVMTG |
| | | | IAANCVYDTQ ILTAIAMGVH ALDIQALNGT |
| | | | NQSFHPFIHN SKPHPGQLWA ADQMISLLAN |
| | | | SQLVRDELDG KHDYRDHELI QDRYSLRCLP |
| | | | QYLGPIVDGI SQIAKQIEIE INSVTDNPLI |
| | | | DVDNQASYHG GNFLGQYVGM GMDHLRYYIG |
| | | | LLAKHLDVQI ALLASPEFSN |
| | | | GLPPSLLGNR ERKVNMGLKG LQICGNSIMP |
| | | | LLTFYGNSIA DRFPTHAEQF NQNINSQGYT |
| | | | SATLARRSVD IFQNYVAIAL MFGVQAVDLR |
| | | | TYKKTGHYDA RACLSPATER LYSAVRHVVG |
| | | | QKPTSDRPYI WNDNEQGLDE HIARISADIA |
| | | | AGGVIVQAVQ DILPCLH |
| 19 | Human T- | Uniprot | MVLSQEEPDS ARGTSEAQPL GPAPTGAAPP |
| | type | Q8TF71 | PGPGPSDSPE AAVEKVEVEL AGPATAEPHE |
| | amino- | | PPEPPEGGWG WLVMLAAMWC NGSVFGIQNA |
| | acid | | CGVLFVSMLE |
| | transporter-1 | | TFGSKDDDKM VFKTAWVGSL SMGMIFFCCP |
| | (TAT1) | | IVSVFTDLFG CRKTAVVGAA VGFVGLMSSS |
| | | | FVSSIEPLYL TYGIIFACGC SFAYQPSLVI |
| | | | LGHYFKKRLG LVNGIVTAGS SVFTILLPLL |
| | | | LRVLIDSVGL FYTLRVLCIF MFVLFLAGFT |
| | | | YRPLATSTKD KESGGSGSSL FSRKKFSPPK |
| | | | KIFNFAIFKV TAYAVWAVGI PLALFGYFVP |
| | | | YVHLMKHVNE RFQDEKNKEV VLMCIGVTSG |
| | | | VGRLLFGRIADYVPGVKKVY LQVLSFFFIG |
| | | | LMSMMIPLCS IFGALIAVCL IMGLFDGCFI SIMAPIAFEL |
| | | | VGAQDVSQAI GFLLGFMSIP MTVGPPIAGL |
| | | | LRDKLGSYDV AFYLAGVPPL IGGAVLCFIP |
| | | | WIHSKKQREI SKTTGKEKME KMLENQNSLL |
| | | | SSSSGMFKKE SDSII |
| 20 | Human | Uniprot | MPSETPQAEV GPTGCPHRSG PHSAKGSLEK |
| | Cystathionine | P35520 | GSPEDKEAKE PLWIRPDAPS RCTWQLGRPA |
| | B-synthase | | SESPHHHTAP AKSPKILPDI LKKIGDTPMV |
| | | | RINKIGKKFG LKCELLAKCE FFNAGGSVKD |
| | | | RISLRMIEDA ERDGTLKPGD TIIEPTSGNT |
| | | | GIGLALAAAV RGYRCIIVMP EKMSSEKVDV |
| | | | LRALGAEIVR TPTNARFDSP |
| | | | ESHVGVAWRL KNEIPNSHIL DQYRNASNPL |
| | | | AHYDTTADEI LQQCDGKLDM LVASVGTGGT |
| | | | ITGIARKLKE KCPGCRIIGV DPEGSILAEP |
| | | | EELNQTEQTT YEVEGIGYDF IPTVLDRTVV |
| | | | DKWFKSNDEE AFTFARMLIA QEGLLCGGSA |
| | | | GSTVAVAVKA AQELQEGQRC VVILPDSVRN |
| | | | YMTKFLSDRW MLQKGFLKEE |
| | | | DLTEKKPWWW HLRVQELGLS APLTVLPTIT |
| | | | CGHTIEILRE KGFDQAPVVD EAGVILGMVT |
| | | | LGNMLSSLLA GKVQPSDQVG KVIYKQFKQI |
| | | | RLTDTLGRLS HILEMDHFAL VVHEQIQYHS |
| | | | TGKSSQRQMV FGVVTAIDLL NFVAAQERDQ K |
| 21 | Human | Uniprot | MKKAEMGRFS ISPDEDSSSY SSNSDFNYSY |
| | SAT2 | Q96QD8 | PTKQAALKSH YADVDPENQN FLLESNLGKK |
| | | | KYETEFHPGT TSFGMSVFNL SNAIVGSGIL |
| | | | GLSYAMANTG IALFIILLTF VSIFSLYSVH |
| | | | LLLKTANEGG SLLYEQLGYK AFGLVGKLAA |
| | | | SGSITMQNIG AMSSYLFIVK YELPLVIQAL |
| | | | TNIEDKTGLW YLNGNYLVLL |
| | | | VSLVVILPLS LFRNLGYLGY TSGLSLLCMV |
| | | | FFLIVVICKK FQVPCPVEAA LIINETINTT LTQPTALVPA |
| | | | LSHNVTENDS CRPHYFIFNS QTVYAVPILI |
| | | | FSFVCHPAVL PIYEELKDRS RRRMMNVSKI |
| | | | SFFAMFLMYL LAALFGYLTF YEHVESELLH |
| | | | TYSSILGTDI LLLIVRLAVL MAVTLTVPVV IFPIRSSVTH |
| | | | LLCASKDFSW WRHSLITVSI LAFTNLLVIF VPTIRDIFGF |
| | | | IGASAASMLI FILPSAFYIK LVKKEPMKSV |
| | | | QKIGALFFLL SGVLVMTGSM ALIVLDWVHN PGGGH |
| 22 | Human | Uniprot | MEKSNETNGY LDSAQAGPAA GPGAPGTAAG |
| | ASCT1 | P43007 | RARRCAGFLR RQALVLLTVS GVLAGAGLGA |
| | | | ALRGLSLSRT QVTYLAFPGE MLLRMLRMII |
| | | | LPLVVCSLVS GAASLDASCL GRLGGIAVAY |
| | | | FGLTTLSASA LAVALAFIIK PGSGAQTLQS |
| | | | SDLGLEDSGP PPVPKETVDS FLDLARNLFP |
| | | | SNLVVAAFRT YATDYKVVTQ NSSSGNVTHE |
| | | | KIPIGTEIEG MNILGLVLFA LVLGVALKKL |
| | | | GSEGEDLIRF FNSLNEATMV LVSWIMWYVP |
| | | | VGIMFLVGSK IVEMKDIIVL VTSLGKYIFA |
| | | | SILGHVIHGG IVLPLIYFVF TRKNPFRFLL GLLAPFATAF |
| | | | ATCSSSATLP SMMKCIEENN GVDKRISRFI |
| | | | LPIGATVNMD GAAIFQCVAA VFIAQLNNVE |
| | | | LNAGQIFTIL VTATASSVGA AGVPAGGVLT |
| | | | IAIILEAIGL PTHDLPLILA VDWIVDRTTT |
| | | | VVNVEGDALG AGILHHLNQK ATKKGEQELA |
| | | | EVKVEAIPNC KSEEETSPLV THQNPAGPVA |
| | | | SAPELESKES VL |
| 23 | Aspergillus | Uniprot | MSAVKAARYG KDNVRVYKVH KDEKTGVQTV |
| | flavus | Q00511 | YEMTVCVLLE GEIETSYTKA DNSVIVATDS |
| | uricase | | IKNTIYITAK QNPVTPPELF GSILGTHFIE |
| | (AfUricase) | | KYNHIHAAHV NIVCHRWTRM DIDGKPHPHS |
| | | | FIRDSEEKRN VQVDVVEGKG IDIKSSLSGL |
| | | | TVLKSTNSQF WGFLRDEYTT LKETWDRILS |
| | | | TDVDATWQWK NFSGLQEVRS |
| | | | HVPKFDATWA TAREVTLKTF AEDNSASVQA |
| | | | TMYKMAEQIL ARQQLIETVE YSLPNKHYFE |
| | | | IDLSWHKGLQ NTGKNAEVFA PQSDPNGLIK |
| | | | CTVGRSSLKS KL |
| 24 | Human | Uniprot | MADSRDPASD QMQHWKEQRA AQKADVLTTG |
| | catalase | P04040 | AGNPVGDKLN VITVGPRGPL LVQDVVFTDE |
| | | | MAHFDRERIP ERVVHAKGAG AFGYFEVTHD |
| | | | ITKYSKAKVF EHIGKKTPIA VRFSTVAGES |
| | | | GSADTVRDPR GFAVKFYTED GNWDLVGNNT |
| | | | PIFFIRDPIL FPSFIHSQKR NPQTHLKDPD |
| | | | MVWDFWSLRP ESLHQVSFLF SDRGIPDGHR |
| | | | HMNGYGSHTF KLVNANGEAV YCKFHYKTDQ |
| | | | GIKNLSVEDA ARLSQEDPDY GIRDLFNAIA |
| | | | TGKYPSWTFY IQVMTFNQAE TFPFNPFDLT |
| | | | KVWPHKDYPL IPVGKLVLNR NPVNYFAEVE |
| | | | QIAFDPSNMP PGIEASPDKM |
| | | | LQGRLFAYPD THRHRLGPNY LHIPVNCPYR |
| | | | ARVANYQRDG PMCMQDNQGG APNYYPNSFG |
| | | | APEQQPSALE HSIQYSGEVR RFNTANDDNV |
| | | | TQVRAFYVNV LNEEQRKRLC ENIAGHLKDA |
| | | | QIFIQKKAVK NFTEVHPDYG SHIQALLDKY |
| | | | NAEKPKNAIH TFVQSGSHLA AREKANL |
| 25 | Coagulation | Uniprot | ANSFLEEMKKGHLERECMEETCSYEEAREVFEDSDKTN |
| | Factor X | P00742 | EFWNKYKDGDQCETSPCQNQGKCKD |
| | | | GLGEYTCTCLEGFEGKNCELFTRKLCSLDNGDCDQFCH |
| | | | EEQNSVVCSCARGYTLADNGKACIPTG |
| | | | PYPCGKQTLERRKRSVAQATSSSGEAPDSITWKPYDAA |
| | | | DLDPTENPFDLLDFNQTQPERGDNNL |
| | | | TRIVGGQECKDGECPWQALLINEENEGFCGGTILSEFYIL |
| | | | TAAHCLYQAKRFKVRVGDRNTEQEEG |
| | | | GEAVHEVEVVIKHNRFTKETYDFDIAVLRLKTPITFRMN |
| | | | VAPACLPERDWAESTLMTQKTGIVSGF |
| | | | GRTHEKGRQSTRLKMLEVPYVDRNSCKLSSSFIITQNMF |
| | | | CAGYDTKQEDACQGDSGGPHVTRFK |
| | | | DTYFVTGIVSWGEGCARKGKYGIYTKVTAFLKWIDRS |
| | | | MKTRGLPKAKSHAPEVITSSPLK |
| 26 | Coagulation | Uniprot | ANAFLEELRPGSLERECKEEQCSFEEAREIFKDAERTKLF |
| | Factor VII | P08709 | WISYSDGDQCASSPCQNGGSCKDQLQ |
| | | | SYICFCLPAFEGRNCETHKDDQLICVNENGGCEQYCSD |
| | | | HTGTKRSCRCHEGYSLLADGVSCTPTVE |
| | | | YPCGKIPILEKRNASKPQGRIVGGKVCPKGECPWQVLLL |
| | | | VNGAQLCGGTLINTIWVVSAAHCFDKI |
| | | | KNWRNLIAVLGEHDLSEHDGDEQSRRVAQVIIPSTYVP |
| | | | GTTNHDIALLRLHQPVVLTDHVVPLCLP |
| | | | ERTFSERTLAFVRFSLVSGWGQLLDRGATALELMVLNV |
| | | | PRLMTQDCLQQSRKVGDSPNITEYMF |
| | | | CAGYSDGSKDSCKGDSGGPHATHYRGTWYLTGIVSWG |
| | | | QGCATVGHFGVYTRVSQYIEWLQKL |
| | | | MRSEPRPGVLLRAPFP |
| 27 | Anti-TFPI | IMGT | EVQLVESGGGLVKPGGSLRLSCAASGFTFSNYAMSWV |
| | scFv 1 | INN | RQTPEKRLEWVATISRSGSYSYFP |
| | | 9636 | DSVQGRFTISRDNAKNSLYLQMNSLRAEDTAVYYCARL |
| | | | GGYDEGDAMDSWGQGTTVTV |
| | | | SSGGGGSGGGGSGGGGSDIVMTQTPLSLSVTPGQPASIS |
| | | | CKSSQSLLESDGKTYLNWYLQ |
| | | | KPGQSPQLLIYLVSILDSGVPDRFSGSGSGTDFTLKISRV |
| | | | EAEDVGVYYCLQATHFPQTFGG |
| | | | GTKVEIK |
| 28 | Anti-TFPI | | EVQLLESGGGLVQPGGSLRLSCAASGFTFSSYAMSWVR |
| | scFv 2 | | QAPGKGLEWVSAISGSGGSTYY |
| | | | ADSVKGRFTISRDNSKNTLYLQMNSLRAEDTAVYYCAI |
| | | | LGATSLSAFDIWGQGTMVTVSSG |
| | | | GGGSGGGGSGGGGSQSVLTQPPSVSGAPGQRVTISCTG |
| | | | SSSNIGAGYDVHWYQQLPGTA |
| | | | PKLLIYGNSNRPSGVPDRFSGSKSGTSASLAITGLQAEDE |
| | | | ADYYCQSYDSSLSGSGVFGGGTK |
| | | | LTVL |
| 29 | Anti- | IMGT | DIQMTQSPSSLSASVGDRVTITCKASRNIERQLAWYQQK |
| | FIXa/FX | INN | PGQAPELLIYQASRKESGVPDR |
| | scFv 1 | 10115 | FSGSRYGTDFTLTISSLQPEDIATYYCQQYSDPPLTFGGG |
| | | | TKVEIKGGGGSGGGGSGGGGS |
| | | | QVQLVQSGSELKKPGASVKVSCKASGYTFTDNNMDWV |
| | | | RQAPGQGLEWMGDINTRSGG |
| | | | SIYNEEFQDRVIMTVDKSTDTAYMELSSLRSEDTATYHC |
| | | | ARRKSYGYYLDEWGEGTLVTVSS |
| 30 | Anti- | IMGT | DIQMTQSPSSLSASVGDRVTITCKASRNIERQLAWYQQK |
| | FIXa/FX | INN | PGQAPELLIYQASRKESGVPDR |
| | scFv 2 | 10115 | FSGSRYGTDFTLTISSLQPEDIATYYCQQYSDPPLTFGGG |
| | | | TKVEIKGGGGSGGGGSGGGGS |
| | | | QVQLVESGGGLVQPGGSLRLSCAASGFTFSYYDIQWVR |
| | | | QAPGKGLEWVSSISPSGQSTYY |
| | | | RREVKGRFTISRDNSKNTLYLQMNSLRAEDTAVYYCAR |
| | | | RTGREYGGGWYFDYWGQGTLV |
| | | | TVSS |
| 51 | Myeloper | UniProt | MGVPFFSSLR CMVDLGPCWA GGLTAEMKLL |
| | oxidase | P05164 | LALAGLLAIL ATPQPSEGAA |
| | (MPO) | | PAVLGEVDTS LVLSSMEEAK QLVDKAYKER |
| | | | RESIKQRLRS GSASPMELLS |
| | | | YFKQPVAATR TAVRAADYLH VALDLLERKL |
| | | | RSLWRRPFNV TDVLTPAQLN |
| | | | VLSKSSGCAY QDVGVTCPEQ DKYRTITGMC |
| | | | NNRRSPTLGA SNRAFVRWLP |
| | | | AEYEDGFSLP YGWTPGVKRN GFPVALARAV |
| | | | SNEIVRFPTD QLTPDQERSL |
| | | | MFMQWGQLLD HDLDFTPEPA ARASFVTGVN |
| | | | CETSCVQQPP CFPLKIPPND |
| | | | PRIKNQADCI PFFRSCPACP GSNITIRNQI NALTSFVDAS |
| | | | MVYGSEEPLA |
| | | | RNLRNMSNQL GLLAVNQRFQ DNGRALLPFD |
| | | | NLHDDPCLLT NRSARIPCFL |
| | | | AGDTRSSEMP ELTSMHTLLL REHNRLATEL |
| | | | KSLNPRWDGE RLYQEARKIV |
| | | | GAMVQIITYR DYLPLVLGPT AMRKYLPTYR |
| | | | SYNDSVDPRI ANVFTNAFRY |
| | | | GHTLIQPFMF RLDNRYQPME PNPRVPLSRV |
| | | | FFASWRVVLE GGIDPILRGL |
| | | | MATPAKLNRQ NQIAVDEIRE RLFEQVMRIG |
| | | | LDLPALNMQR SRDHGLPGYN |
| | | | AWRRFCGLPQ PETVGQLGTV LRNLKLARKL |
| | | | MEQYGTPNNI DIWMGGVSEP |
| | | | LKRKGRVGPL LACIIGTQFR KLRDGDRFWW |
| | | | ENEGVFSMQQ RQALAQISLP |
| | | | RIICDNTGIT TVSKNNIFMS NSYPRDFVNC |
| | | | STLPALNLAS WREAS |
| 52 | anti-PCSK9 | | EVQLVQSGAEVKKPGASVKVSCKASGYTLTSYGISWVR |
| | | | QAPGQGLEWMGWVSFYNGNTNYAQKLQGRGTMTTDP |
| | | | STSTAYMELRSLRSDDTAVYYCARGYGMDVWGQGTT |
| | | | VTVSSGGGGSGGGGSGGGGSESALTQPASVSGSPGQSIT |
| | | | ISCTGTSSDVGGYNSVSWYQQHPGKAPKLMIYEVSNRP |
| | | | SGVSNRFSGSKSGNTASLTISGLQAEDEADYYCNSYTST |
| | | | SMVFGGGTKLTVL |

TABLE 4
Exemplary modifiers, e.g., proteases

| | |
|---|---|
| Modifier | Exemplary target |

Proteases

| | |
|---|---|
| IdeS | IgG |
| IdeZ (an immunoglobulin-degrading enzyme from | IgG |
| Streptococcus equi subspecies zooepidemicus) | |
| IgA protease | IgG |
| Papain | IgG |
| ADAM17/TACE | TNF-alpha |
| mesotrypsin | Peptides comprising linkages involving |
| | the carboxyl group of lysine or arginine |
| Lysozyme | peptidoglycan |
| Endolysin | peptidoglycan |
| Endoproteinase, e.g., LysC (can cleave proteins on | Protein having a Lys-Xaa motif |
| C-terminal side of lysine residues) | |
| Metalloendopeptidase, e.g., LysN (can cleave | Protein having an Xaa-Lys motif |
| proteins on amino side of lysine residues) | |
| Elastase, e.g., Pseudomonas elastase (PaE) | C3 |
| alkaline protease (PaAP) | C3 |
| 56 kDa protease from Serratia marcescens | C5a, C1-INH, alpha 2-antiplasmin, |
| | antithrombin III |
| C5a peptidase, e.g., Streptocoocal C5a peptidase, | C5a |
| ScpA, ScpB, SCPA | |
| Plasmin | IgG, C3b, iC3b |
| cysteine protease, e.g., Streptococcal pyrogenic | IgG, cytokines, extracellular matrix |
| exotoxin B (SpeB) | proteins |
| PrtH (e.g., from Porphyromonas) | IgG or C3 |
| Staphylokinase | plasminogen, IgG, C3b |
| Matrix metalloproteinases (e.g., MMP1, MMP2, | ECM proteins, e.g., collagen, gelatin, |
| MMP3, MMP7, MMP8, MMP9, MMP10, MMP11, | fibronectin, laminin, aggrecan, elastin, |
| MMP12, MMP13, MMP14, MMP15, MMP16, | fibrin |
| MMP17, MMP19, MMP20, MMP21, MMP23A, | |
| MMP23B, MMP24, MMP25, MMP26, MMP27, | |
| MMP28) | |

Other modifiers

| | |
|---|---|
| Protein disulfide isomerases | Proteins comprising two cysteine residues |
| Glycosyltransferases, e.g., α-glucan-branching | Protein comprising tyrosine, serine, |
| glycosyltransferase, enzymatic branching factor, | threonine, or asparagine glycosylation site |
| branching glycosyltransferase, enzyme Q, glucosan | |
| transglycosylase, glycogen branching enzyme, | |
| amylose isomerase, plant branching enzyme, α-1,4- | |
| glucan: α-1,4-glucan-6-, glycosyltransferase, starch | |
| branching enzyme, UDP-N-acetyl-D-galactosamine, | |
| polypeptide, N-acetylgalactosaminyltransferase, | |
| GDP-fucose protein O-fucosyltransferase 2, O- | |
| GlcNAc transferase | |
| Acetyltransferases or deacetylases, e.g., | histone |
| nucleosome-histone acetyltransferase, histone | |
| acetokinase, histone acetylase, histone | |
| transacetylase, histone deacetylase | |
| Acyltransferases | Protein comprising an acyl group |
| Phosphatases, e.g., protein-tyrosine-phosphatase, | phosphoprotein |
| phosphotyrosine phosphatase, phosphoprotein | |
| phosphatase (phosphotyrosine), phosphotyrosine | |
| histone phosphatase, protein phosphotyrosine | |
| phosphatase, tyrosylprotein phosphatase, | |
| phosphotyrosine protein phosphatase, | |
| phosphotyrosylprotein phosphatase, tyrosine O- | |
| phosphate phosphatase, PPT-phosphatase, PTPase, | |
| [phosphotyrosine]protein phosphatase, PTP- | |
| phosphatase | |
| Kinases, e.g., non-specific serine/threonine protein | Protein comprising a serine or threonine |
| kinase, Fas-activated serine/threonine kinase, | phosphorylation site |
| Goodpasture antigen-binding protein kinase, IκB | |
| kinase, cAMP-dependent protein kinase, cGMP- | |
| dependent protein kinase, protein kinase C, polo | |
| kinase, cyclin-dependent kinase, mitogen-activated | |
| protein kinase, mitogen-activated protein kinase | |
| kinase kinase, receptor protein serine/threonine | |
| kinase, dual-specificity kinase | |
| Gamma-carboxylases | Protein comprising glutamic acid |
| Methyltransferases | Protein comprising a lysine methylation |
| | site; DNA; RNA |
| Complement-factor inactivating moiety, e.g., | Complement factor, e.g., C1, C2a, C4b, |
| complement control protein, Factor H or Factor I | C3, C3a, C3b, C5, C5a, C5b, C6, C7, C8, |
| | or C9 |

TABLE 6
Exemplary first exogenous polypeptides that interact with a target

| Polypeptide | Exemplary target(s) |
|---|---|
| CD16A | IgG Fc |
| CD14 | LPS |
| TLR4 | LPS |
| scFv | Complement (e.g., C3 or C5) or cytokine (e.g., |
| | TNF-alpha or IL-6 or another cytokine of |
| | Table 1) |
| Aquaporin 4 (AQP4) and variants | Anti-AQP4 autoantibodies |
| Phospholipase A2 receptor (PLA2R) and | Anti-PLA2R autoantibodies |
| variants (shortened domains & peptides) | |
| Acetylcholine receptor (AChR) and variants | Anti-AChR autoantibodies |
| (shortened domains & peptides) | |
| Insulin and variants (proinsulin, preproinsulin, | Anti-insulin autoantibodies |
| etc.) | |
| B2-glycoprotein 1 (b2GP1) and variants | Anti-b2GP1 autoantibodies |
| (shortened domains & peptides) | |
| ADAMTS13 and variants (shortened domains | Anti-ADAMTS13 autoantibodies |
| & peptides) | |
| GAD65 and variants (shortened domains & | Anti-GAD65 autoantibodies |
| peptides) | |
| Desmogleins, e.g., Desmoglein-3 or | Anti-Desmoglein autoantibodies |
| Desmoglein-1 and variants (shortened domains | |
| & peptides) | |
| Complement-factor binding moiety, e.g., CD55 | Complement factor, e.g., C1, C2a, C4b, C3, |
| or CD46 | C3a, C3b, C5, C5a, C5b, C6, C7, C8, or C9 |

### EXAMPLES

### Example 1. Agent-Synergistic Activity of Enucleated Erythroid Cells Expressing Two Different TRAIL Receptor Ligands on the Surface

This Example describes the construction of enucleated erythroid cells comprising pro-apoptotic polypeptides. This type of cell can be used, e.g., to promote apoptosis of cells that promote inflammation, e.g., in a subject having a cardiovascular disease with an inflammatory component.

The genes for TRAIL receptor agonists DR4.2 (SEQ ID NO: 44) and DR5.2 (SEQ ID NO: 47) were synthesized. The genes were cloned into a lentivirus vector (SBI) upstream of the gene for human glycophorin A and separated by a sequence encoding a 12-amino acid Gly-Ser (GGGSGGGSGGGS (SEQ ID NO: 50)) flexible linker and an HA epitope tag (YPYDVPDY (SEQ ID NO: 51)).

Human CD34+ cells derived from mobilized peripheral blood cells from normal human donors were purchased frozen from AllCells Inc. Cells were thawed in PBS with 1% FBS. Cells were then cultured in StemSpan SFEM media with StemSpan CC100 Cytokine Mix at a density of 1E5 cells/mL. Media was swapped to differentiation media on day 5.

Virus production protocol was conducted as follows. Briefly, HEK293T cells were seeded 24 hours before transfection. Cells were transfected with lentivector containing the construct along with packaging plasmids. A media swap was performed 24 hours after transfection and viruses were harvested 72 hours after transfection. On day 6 after thaw, cells were transduced with equal volumes of each virus in a 1:1 cell volume to virus volume ratio, and spinoculated at 845×g for 1.5 hours with 5-10 μg/ml of polybrene.

Transduced cells were differentiated in defined media to erythroid lineage cells and to mature enucleated reticulocytes following the method of Hu et al., Blood 18 Apr. 2013 Vol 121, 16. In brief, the cell culture procedure was comprised of 3 phases. Composition of the base culture medium was Iscove's Modified Dulbecco's Medium, 2% human peripheral blood plasma, 3% human AB serum, 200 mg/mL Holohuman transferrin, 3 IU/mL heparin, and 10 mg/mL insulin. In the first phase (day 0 to day 6), CD34+ cells at a concentration of 10\hat{ }5/mL were cultured in the presence of 10 ng/mL stem cell factor, 1 ng/mL IL-3, and 3 IU/mL erythropoietin. In the second phase (day 7 to day 11), IL-3 was omitted from the culture medium. In the third phase that lasted until day 21, the cell concentration was adjusted to 10\hat{ }6/mL on day 11 and to 5×10\hat{ }6/mL on day 15, respectively. The medium for this phase was the base medium plus 3 IU/mL erythropoietin, and the concentration of transferrin was adjusted to 1 mg/mL.

Expression of the transgenes was monitored by labeling with soluble TRAIL R1 and TRAIL R2 (purchased from Sigma-Aldrich Inc.) that had been chemically conjugated to complementary fluorescent dyes Fluorescein and DyLight 650 and staining by flow cytometry. Expression levels of both ligands DR4.2 and DR5.2 were verified through flow cytometry.

An apoptosis assay was conducted according to a modified version of Marconi et al., Cell Death and Disease 2013. In short, fully mature enucleated reticulocytes expressing DR4.2 and DR5.2 were incubated with CFSE-labeled Raji Cells for 24 hours at a 1:1 ratio. Afterwards, cells were stained with annexin V and analyzed by flow cytometry. Apoptosis percentages were determined from CFSE positive Raji cells that also stained positive for annexin V.

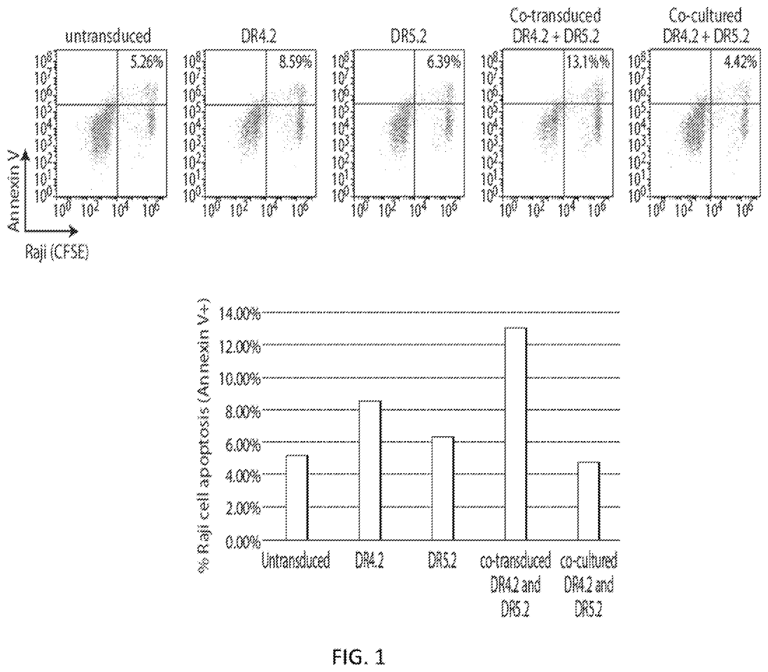

As shown in FIG. 1, when CFSE-labeled Raji cells were incubated with untransduced, DR4.2 transduced, DR5.2 transduced, or a mixture of DR4.2 transduced and DR5.2 transduced cultured reticulocytes, minimal cell death was observed over background. However, when CFSE-labeled Raji cells were incubated with cultured reticulocytes that had been simultaneously transduced with both DR4.2 and DR5.2 and thus express both proteins simultaneously, a significant amount of cell death was observed (equivalent to the maximal amount of TRAIL-induced apoptosis achievable in this assay with Raji cells—see, e.g. Marconi et al., Cell Death and Disease 2013). This data indicates that the coordinated action of TRAIL receptor agonists on the surface of a single engineered erythroid cell is able to induce cell killing in a synergistic manner, relative to cells expressing single TRAIL receptor agonists and even a mixture of cells that each express a different TRAIL receptor agonist.

### Example 2. Generation of Enucleated Erythroid Cells Comprising GLP-1 and Insulin for Use in Treating Diabetes

The genes for human GLP-1 (SEQ ID NO: 1) and human insulin (SEQ ID NO: 2) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)).

Human CD34+ cells derived from mobilized peripheral blood cells from normal human donors are purchased frozen from AllCells Inc. Cells are thawed and cultured at a density of 1E5 cells/mL. Conditions for culturing CD34+ cells are described, e.g., in WO2015/073587 which is herein incorporated by reference in its entirety.

The virus production protocol is conducted by seeding HEK293T cells 24 hours before transfection. Cells are transfected with lentivector containing the construct along with packaging plasmids, and viruses are harvested. Cells are then transduced with the lentivirus. Lentivirus transduction is described, e.g., in WO2015/073587. Transduced cells are differentiated in defined media to erythroid lineage cells and to enucleated reticulocytes, e.g., as described in WO2015/073587.

Expression of the transgenes is monitored by labeling with anti-GLP1 and anti-insulin that had been chemically conjugated to complementary fluorescent dyes Fluorescein and DyLight 650 and staining by flow cytometry. Expression levels of both ligands GLP-1 and insulin are verified through flow cytometry.

### Example 3. Activity of Enucleated Erythroid Cells Comprising GLP-1 and Insulin in a Murine Diabetes Model

The enucleated erythroid cells comprising murine GLP-1 and insulin are prepared using the murine sequences for each peptide as described in Example 2. The resultant enucleated erythroid cells are then formulated in a buffer appropriate for erythroid cells and a dose of between 1e6 and 1e10 enucleated erythroid cells is administered intravenously to the Apoe-/-mouse model as described in, e.g., Hirano and Mori, J Diabetes Investig. 7: 80-86, 2016, which is herein incorporated by reference in its entirety. Administration of the enucleated erythroid cells comprising murine GLP-1 and insulin may result in reduced monocyte adhesion to the endothelium of aorta and suppressed atherosclerotic lesions in apolipoprotein E knockout (Apoe -/-) mice compared to untreated mice using the methods as described in, e.g., Arikawa et al Diabetes. 59(4): 1030-1037, 2010, which is herein incorporated by reference in its entirety.

### Example 4. Generation of Enucleated Erythroid Cells Comprising FGF-21 and Glucagon for use in Treating Obesity

The genes for human FGF-21 (SEQ ID NO: 5; Uniprot Q9NSA1) and human glucagon (SEQ ID NO: 6; Uniprot P01275) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The erythroid cell expressing surface FGF-21 and glucagon fusions are produced as described in Example 2.

### Example 5. Activity of Enucleated Erythroid Cells Comprising FGF-21 and Glucagon in a Murine Type 2 Diabetes Model

The enucleated erythroid cells comprising murine FGF-21 and glucagon are prepared using the murine sequences for each peptide as described in Example 4. The resultant enucleated erythroid cells are then formulated in a buffer appropriate for erythroid cells. Administration of the enucleated erythroid cells comprising murine FGF-21 and glucagon may result in the murine C57BL/7-DIO (diet-induced obesity) model, which is used to test type 2 diabetes and obesity drugs due to its similarities with human type 2 diabetes. Activity in this model is shown by a decrease blood glucose levels relative to controls as described in Wang and Liao Methods Mol Biol. 821:421-433, 2012 which is herein incorporated by reference in its entirety. Glucose tolerance tests, insulin level measurement, glucose stimulated insulin secretion, and insulin tolerance tests may also be used to determine efficacy of the treatment in this diabetes mouse model.

### Example 6. Generation of Enucleated Erythroid Cells Comprising Leptin and Glucagon for Use in Treating Obesity

The genes for human leptin (SEQ ID NO: 7; Uniprot P41159) and human glucagon (SEQ ID NO: 6; Uniprot P01275) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The erythroid cell comprising surface leptin and glucagon fusions are produced as described in Example 2.

### Example 7. Activity of Enucleated Erythroid Cells Comprising Leptin and Glucagon in a Murine Obesity Model

The enucleated erythroid cells comprising murine leptin and glucagon are made as described in Example 6. The resultant enucleated erythroid cells are then formulated in a buffer appropriate for erythroid cells. The murine ob/ob model, which is a homozygous spontaneous knock out of the leptin gene, develops a phenotype similar to human obesity, as described in Houseknecht and Portocarrero Domestic Animal Endocrinology. 15(6):457-475, 1998 which is herein incorporated by reference in its entirety. Administration of active enucleated erythroid cells comprising leptin and glucagon relative to controls, decreases obesity readouts. Readouts include whole body weight, end point fat mass, liver steatosis, food intake, insulin levels, glucose tolerance tests, insulin tolerance tests.

### Example 8. Generation of Enucleated Erythroid Cells Comprising BNP and Relaxin for Use in Treating, e.g., Acute Heart Failure

The genes for BNP (SEQ ID NO: 8) and relaxin (SEQ ID NO: 9) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The erythroid cell expressing surface BNP and relaxin fusions are produced as described in Example 2.

### Example 9. Activity of Enucleated Erythroid Cells Comprising BNP and Relaxin in a Swine Myocardial Infarction Model

The enucleated erythroid cells comprising porcine BNP and relaxin are made as described in Example 8. The resultant enucleated erythroid cells are then formulated in a buffer appropriate for erythroid cells. Administration of the enucleated erythroid cells comprising BNP and relaxin, e.g., at reperfusion upon 30 min of ischemia, in a swine model of myocardial infarction, which is currently used to test cardiotropic drugs due to its similarities with human ischemic heart disease, may reduce serum and tissue markers of myocardial injury as described in Bani et al, Ann N Y Acad Sci. 1041:423-30, 2005, and Nistri et al, Pharmacol Res. 57(1):43-8, 2008; each of which are herein incorporated by reference in its entirety. The administration of active enucleated erythroid cells comprising BNP and relaxin reduces plasma histamine, increases cardiac histamine content and decreases cardiac mast cell degranulation, tissue malondialdehyde, cardiomyocyte apoptosis and leukocyte recruitment, e.g., relative to a control. In addition, measures of overall cardiac performance such as the occurrence of severe ventricular arrhythmias can be reduced.

### Example 10. Generation of Enucleated Erythroid Cells Comprising tPA and TFPI for Use in Treating Thromboembolism or Atherosclerosis

The gene for a tissue plasminogen activators (e.g., SEQ ID NO: 10 Uniprot P00750; SEQ ID NO: 11, SEQ ID NO: 12, or SEQ ID NO: 13) and the gene for tissue factor pathway inhibitor (SEQ ID NO: 14; uniprot P10646) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The erythroid cell expressing surface tPA and TFPI fusions are produced as described in Example 2.

### Example 11. Activity of Enucleated Erythroid Cells Comprising tPA and TFPI in a Murine Thromboembolic Stroke Model

The enucleated erythroid cells expressing murine tPA and TFPI are made as described in Example 10. The resultant enucleated erythroid cells are then formulated in a buffer appropriate for erythroid cells. Administration of the enucleated erythroid cells comprising tPA and TFPI will decrease blood clots in a murine model of in situ thromboembolic stroke with purified murine thrombin, e.g., relative to controls, as described in Orset et al. Stroke. 38:2771-2778, 2007 and Garcia-Yebenes et al. Stroke. 42:196-203, 2011; each of which is herein incorporated by reference in its entirety. Improvements in infarct volume, neurological deficit, edema, hemorrhagic area, and blood volume may result following administration of the enucleated erythroid cells comprising tPA and TFPI.

### Example 12. Generation of Enucleated Erythroid Cells Comprising a RANK-L Antibody and Parathyroid Hormone (PTH) for Use in Treating Thromboembolism or Atherosclerosis

The gene for the scFv portion of an antibody blocking RANK-L, such as denosumab, (derived from SEQ ID NOs: 15 and 16) and parathyroid hormone (SEQ ID NO: 17) is synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The erythroid cell expressing comprising Anti-RANK-L scFv and PTH are produced as described in Example 2.

### Example 13. Activity of Enucleated Erythroid Cells Comprising a RANK-L Antibody and PTH in a Murine Thromboembolic Stroke Model

The enucleated erythroid cells expressing a human RANK-L antibody and human PTH may be formulated in a buffer appropriate for erythroid cells. The enucleated erythroid cells comprising a RANK-L antibody and PTH are administered in the ovarectomized cynomolgus monkey model, which is used to test osteoporosis drugs due to its similarities with human osteoporosis. Activity in this assay results in one or more of reductions in osteoclast surface, eroded surface, cortical porosity and fluorochrome labeling along with increases in bone mineral density as described in Kostenuik et al, J Bone Miner Res. 2015 April; 30(4):657-69, while destructive biomechanical testing shows greater vertebral strength in monkeys treated with the cells.

### Example 14. Generation of Enucleated Erythroid Cells Comprising Phenylalanine Ammonia Lyase (PAL) and Phenylalanine Transporter TAT1 for Use in Treating Phenylketonuria (PKU)

The genes for PAL from Anabaena variabilis (AvPAL; SEQ ID NO: 18) and a phenylalanine transporter such as human T-type amino-acid transporter-1 (TAT1; SLC16A10; SEQ ID NO: 19) are synthesized. The genes are cloned into a lentivirus vector (SBI). Human CD34+ cells derived from mobilized peripheral blood cells from normal human donors are purchased frozen from AllCells Inc. Cells are thawed and cultured at a density of 1E5 cells/mL. Conditions for culturing CD34+ cells are described, e.g., in WO2015/073587.

Virus production protocol is conducted as follows. Briefly, HEK293T cells are seeded 24 hours before transfection. Cells are transfected with lentivector containing the construct along with packaging plasmids, and viruses are harvested. Cells are then co-transduced with the lentivirus for PAL and a phenylalanine transporter such as TAT1. Lentivirus transduction is described, e.g., in WO2015/073587. Transduced cells are differentiated in defined media to erythroid lineage cells and to enucleated reticulocytes, e.g., as described in WO2015/073587.

Expression of PAL and phenylalanine transporter is monitored by western blot analysis using antibodies specific for PAL and TAT1.

### Example 15. Activity of Enucleated Erythroid Cells Comprising PAL and TAT1 in a PKU Mouse Model

The enucleated erythroid cells expressing PAL and TAT1 are made as described in Example 14 and prepared using sequences for Anabaena variabilis PAL and murine TAT1. The resultant enucleated erythroid cells are then formulated in a buffer appropriate for erythroid cells and a dose of between le6 and le10 enucleated erythroid cells is administered intravenously to Pah -/- mouse model as described in Shedlovsky et al, Genetics 1993; 134: 1205-1210. Activity in this assay leads to reduction of phenylalanine in mouse plasma compared to untreated mice.

### Example 16. Generation of Enucleated Erythroid Cells Comprising Cystathionine B-Synthase (CBS) and L-Homocysteine and L-Serine Transporters for Use in Treating Homocystinuria

The genes for human CBS (SEQ ID NO: 20) and a homocysteine and serine transporter, such as sodium-coupled neutral amino acid transporter 2 (SAT2; SLC38A2; SEQ ID NO: 21), or neutral amino acid transporter A (ASCT1; SLC1A4; SEQ ID NO: 22) are synthesized. The genes are cloned into a lentivirus vector (SBI).

Human CD34+ cells derived from mobilized peripheral blood cells from normal human donors are purchased frozen from AllCells Inc. Cells are thawed and cultured at a density of 1E5 cells/mL. Conditions for culturing CS34+ cells are described, e.g., in WO2015/073587.

Virus production protocol is conducted as follows. Briefly, HEK293T cells are seeded 24 hours before transfection. Cells are transfected with lentivector containing the construct along with packaging plasmids, and viruses are harvested. Cells are then co-transduced with the lentivirus for CBS and one or more of homocysteine and serine transporters described above. Lentivirus transduction is described, e.g., in WO2015/073587.

Transduced cells are differentiated in defined media to erythroid lineage cells and to enucleated reticulocytes, e.g., as described in WO2015/073587. Expression of CBS and transporters is monitored by western blot analysis using antibodies specific for CBS and transporter.

### Example 17. Activity of Enucleated Erythroid Cells Comprising CBS and Transporter(s) in Homocystinuria Mouse Model

The enucleated erythroid cells expressing CBS and homocysteine, serine transporter(s) such as SAT2 and/or ASCT1 is made as described in Example 16 and prepared using sequences for murine CBS and murine transporter. The resultant enucleated erythroid cells are then formulated in a buffer appropriate for erythroid cells and dose of between 1e6 and 1e10 enucleated erythroid cells is administered intravenously into two different mouse models: 1—murine CBS -/- mouse model as described in Watanabe et al, Proc Natl Acad Sci. 92: 1585-1589, 1995 and 2—murine CBS -/- plus transgenic for human CBS as described in Maclean et al, Mol Genet Metab 2010; 101: 153-162, each of which is herein incorporated by reference in its entirety. Activity in this assay leads to reduction of homocysteine and elevation of cystathionine and cysteine levels in mouse plasma compared to untreated mice using methods described in Bublil et al, J Clin Invest. 26(6): 2372-2384, 2016.

### Example 18. Generation of Enucleated Erythroid Cells Comprising Uricase and Catalase for Use in Treating Hyperuricemia

The genes for Asparagillus flavus uricase (SEQ ID NO: 23) and human catalase (SEQ ID NO: 24) are synthesized. The genes are cloned into a lentivirus vector (SBI).

Human CD34+ cells derived from mobilized peripheral blood cells from normal human donors are purchased frozen from AllCells Inc. Cells are thawed and cultured at a density of 1E5 cells/mL. Conditions for culturing CD34+ cells are described, e.g., in WO2015/073587.

Virus production protocol is conducted as follows. Briefly, HEK293T cells are seeded 24 hours before transfection. Cells are transfected with lentivector containing the construct along with packaging plasmids, and viruses are harvested. Cells are then co-transduced with the lentivirus for uricase and catalase described above. Lentivirus transduction is described, e.g., in WO2015/073587.

Transduced cells are differentiated in defined media to erythroid lineage cells and to enucleated reticulocytes, e.g., as described in WO2015/073587. Expression of uricase and catalase is monitored by western blot analysis using antibodies specific for uricase and catalase.

### Example 19. Activity of Enucleated Erythroid Cells Comprising Uricase and Catalase in Hyperuricemia Mouse Model

The enucleated erythroid cells comprising uricase and catalase is made as described in Example 18 and prepared using sequences for Asparagillus flavus uricase and murine catalase. The resultant enucleated erythroid cells are then formulated in a buffer appropriate for erythroid cells and dose of between 1e6 and 1e10 enucleated erythroid cells is administered intravenously into hyperuricemia mouse model as described in Wu et al, Proc Natl Acad Sci. 91: 742-746, 1994; hereby incorporated by reference in its entirety. Activity in this assay lead to reduction of uric acids levels in mouse plasma compared to untreated mice.

### Example 20. Generation of Enucleated Erythroid Cells Comprising FVIIa and FX for Use in Treating Hemophilia

The genes for FVIIa (SEQ ID NO: 26; Uniprot P08709) and FX (SEQ ID NO: 25; Uniprot P00742) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY) (SEQ ID NO: 4)). The enucleated erythroid cells expressing surface FVIIa and FX fusions are produced as described in Example 2.

### Example 21. Activity of Enucleated Erythroid Cells Comprising FVIIa and FX in a Murine Tail Bleed Model

The enucleated erythroid cells expressing FVIIa and FX is then formulated in a buffer appropriate for erythroid cells. The enucleated erythroid cells expressing FVIIa and FX are administered in the murine tail bleed model used to test hemophilia drugs due to its similarities with the human clotting cascade. Activity in this assay is shown with a reduction in clotting time and blood loss as described in Liu et al, World J Exp Med. 2(2):30-36, 2012, and Ivanciu et al, Nat Biotechnology. 29(11):1028-33, 2012; each of which is incorporated herein by reference in its entirety. Plasma samples collected after administration to mice can be assayed for percent of activity remaining. Markers in plasma at various time points including levels of fibrinogen, platelets, and/or D-dimer can be used to evaluating the safety and efficacy of the treatment.

### Example 22. Generation of Enucleated Erythroid Cells Comprising Anti-FIXa and Anti-FX for Use in Treating Hemophilia

The genes for the scFvs that bind FIXa and FX (SEQ ID NO: 29, IMGT INN 10115; SEQ ID NO: 30; IMGT INN 10115) were synthesized. The genes were cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The erythroid cell expressing surface anti-FIXa and anti-FX fusions were produced as described in Example 2.

The cells were then washed with phosphate buffered saline with 0.1% bovine serum albumin, and stained with an anti-HA antibody linked to a fluorophore (phycoerythrin). The cells were then analyzed via flow cytometry to determine the protein expression. As shown in Table 7, the scFv expression was determined to be in the range of 71.3-87.7%. This experiment indicates that it is possible to express a very high percentage of the cells with two scFvs.

TABLE 7
Expression of anti-FIXa and anti-FX polypeptides from enucleated
erythroid cells as determined via flow cytometry.

| Exogenous proteins | Detection | Percent expression |
|---|---|---|
| Anti-FIXa/FX scFv 1 | HA tag | 71.3% |
| Anti-FIXa/FX scFv 2 | HA tag | 86.6% |
| Anti-FIXa/FX scFv 1 + 2 | HA tag | 87.7% |

### Example 23. Activity of Enucleated Erythroid Cells Comprising Anti-FIXa and Anti-FX in a Murine Tail Bleed Model

The enucleated erythroid cells expressing anti-FIXa and anti-FX is then formulated in a buffer appropriate for erythroid cells. The enucleated erythroid cells expressing anti-FIXa and anti-FX are assayed in the murine tail bleed model currently used to test hemophilia drugs due to its similarities with the human clotting cascade. Activity in this assay results in a reduction in clotting time and blood loss as described in Liu et al, World J Exp Med. 2(2):30-36, 2012, and Ivanciu et al, Nat Biotechnology. 29(11):1028-33, 2011. Plasma samples collected after administration to mice can be assayed for percent of activity remaining. Markers in plasma at various time points including levels of fibrinogen, platelets, and/or D-dimer can be used to evaluating the safety and efficacy of the treatment.

### Example 24. Generation of Enucleated Erythroid Cells Comprising Anti-TFPI and FVIIa for Use in Treating Hemophilia

The genes for anti-TFPI (SEQ ID NO: 27) and FVIIa (SEQ ID NO: 26; Uniprot P08709) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY) (SEQ ID NO: 4)). The enucleated erythroid cells expressing surface anti-TFPI and FVIIa fusions are produced as described in Example 2.

### Example 25. Activity of Enucleated Erythroid Cells Comprising Anti-TFPI and FVIIa in a Murine Tail Bleed Model

The enucleated erythroid cells expressing anti-TFPI and FVIIa are then formulated in a buffer appropriate for erythroid cells. The enucleated erythroid cells are assayed in the murine tail bleed model currently used to test hemophilia drugs due to its similarities with the human clotting cascade. Activity in this assay results in a reduction in clotting time and blood loss as described in Liu et al, World J Exp Med. 2(2):30-36, 2012, and Ivanciu et al, Nat Biotechnology. 29(11):1028-33, 2011. Plasma samples collected after administration to mice can be assayed for percent of activity remaining. Markers in plasma at various time points including levels of fibrinogen, platelets, and/or D-dimer can be used to evaluating the safety and efficacy of the treatment.

### Example 26. Generation of Enucleated Erythroid Cells Comprising Anti-TFPI and FXa for Use in Treating Hemophilia

The genes for anti-TFPI (SEQ ID NO: 27) and FXa (SEQ ID NO: 25; Uniprot P00742) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY) (SEQ ID NO: 4)). The erythroid cell expressing surface anti-TFPI and FXa fusions are produced as described in Example 2.

### Example 27. Activity of Enucleated Erythroid Cells Comprising Anti-TFPI and FXa in a Murine Tail Bleed Model

The enucleated erythroid cells expressing anti-TFPI and FXa is then formulated in a buffer appropriate for erythroid cells. The enucleated erythroid cells are assayed in the murine tail bleed model currently used to test hemophilia drugs due to its similarities with the human clotting cascade. Activity in this assay results in a reduction in clotting time and blood loss as described in Liu et al, World J Exp Med. 2(2):30-36, 2012, and Ivanciu et al, Nat Biotechnology. 29(11):1028-33, 2011. Plasma samples collected after administration to mice can be assayed for percent of activity remaining. Markers in plasma at various time points including levels of fibrinogen, platelets, and/or D-dimer can be used to evaluating the safety and efficacy of the treatment.

### Example 28. Capture and Modification of a Target Protein

In this Example, transgenic enucleated erythroid cells were used to capture and modify a target protein. The control cells and the experimental cells each comprise endogenous glycophorin A (GPA) in their membranes, which was used to bind the target protein. The experimental cells expressed an exogenous protein comprising surface-exposed IdeS (SEQ ID NO: 35) fused to GPA as a membrane anchor. IdeS is capable of cleaving antibodies to produce a F(ab′)2 fragment and a Fc fragment. The target protein is an anti-GPA antibody that is fluorescently labelled with FITC. Both the constant and variable regions of the target antibody were FITC-labelled, so that if the antibody was cleaved, both fragments could be detected.

First, the control cells and IdeS-expressing cells were tested by FACS for the ability to bind the anti-GPA antibody. Both control and IdeS-expressing cells bound the antibody as measured by association of FITC with the cells. In addition, both control and IdeS-expressing cells bound the antibody as measured by or using a second detection method with a fluorescently labeled anti-rabbit Fc antibody. These measurements were taken at an early timepoint, before cells were incubated to allow IdeS-mediated cleavage of the target antibody.

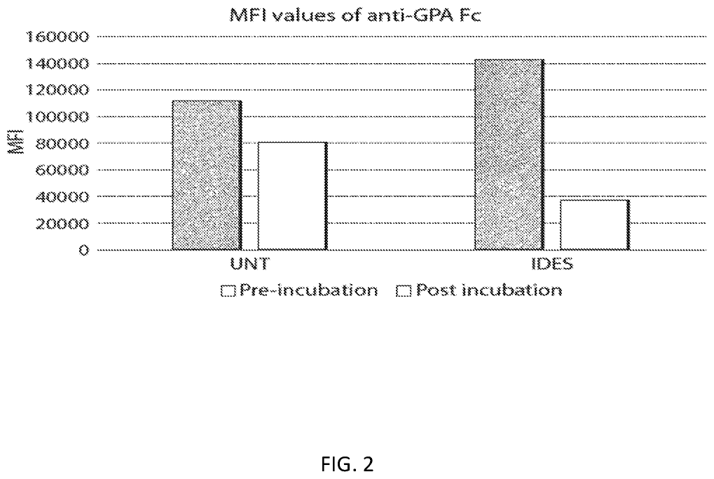

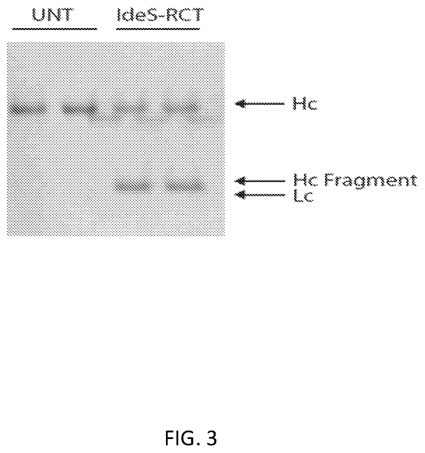

In contrast, only the IdeS-expressing cells were able to cleave the target antibody. This was shown by incubating the control or IdeS-expressing cells with the target antibody to allow antibody cleavage to occur. Fluorescently labeled anti-rabbit Fc antibody was added to the reaction in order to detect intact antibodies on the surface of the erythroid cells. The IdeS-expressing cells showed a decrease in anti-rabbit Fc association with the cells (FIG. 2), indicating lower levels of Fc on the surface of the IdeS-expressing cells compared to the control cells. There was no decrease in the amount of the directly FITC-labeled target antibody associated with control cells or IdeS-expressing cells, indicating that at least the FITC-labeled variable region of the target antibody still bound the IdeS-expressing and control cells. This result was confirmed by Western blot, where anti rabbit heavy chain and anti rabbit light chain antibodies were used to detect intact and cleaved antibody in the supernatant of control or IdeS-expressing cells. The experiment showed that IdeS-expressing erythroid cells but not control erythroid cells cleaved the anti-GPA-antibody, resulting in appearance of the heavy chain fragment (FIG. 3).

Thus, the control cells were able to bind the target antibody, but only the IdeS-expressing cells were able to bind and cleave the target antibody.

### Example 29. Generation of Enucleated Erythroid Cells Comprising Anti-TNFa and Anti-PCSK9 for Use in Treating a Cardiovascular Disease

The genes for anti-TNFa (SEQ ID NO: 49) and anti-PCSK9 (SEQ ID NO: 52) are synthesized by a commercial vendor. The genes are cloned into a lentivirus vector (SBI) upstream of the gene for human glycophorin A and separated by a sequence encoding a 12-amino acid Gly-Ser (GGGSGGGSGGGS (SEQ ID NO: 19)) flexible linker and an HA epitope tag (YPYDVPDY (SEQ ID NO: 20)).

Human CD34+ cells can be cultured, and virus can be produced, as described in Example 5. Transduced cells are differentiated as described herein.

To assess the expression of the transgenes, cells are labeled simultaneously with the ligands anti-TNFa and anti-PCSK9 (purchased from Life Technologies) and lipopolysaccharide (ThermoFisher), which are chemically conjugated to complementary fluorescent dyes. The cells are analyzed by flow cytometry to verify that (a) the agents are all expressed on the surface of the cell and (b) the agents are capable of binding to their target ligands.

The cell population is formulated in AS-3 additive solution and administered intravenously to a patient who is, e.g, developing bacteremia. Administration of the enucleated erythroid cells comprising anti-TNFa and anti-PCSK9 may result in the patient exhibiting an improvement in cardiovascular symptoms as measured by an improvement in a value for a parameter associated with cardiovascular function, e.g., blood pressure.

1. An enucleated erythroid cell, comprising a first exogenous polypeptide comprising a first cardiovascular therapeutic, and a second exogenous polypeptide, comprising a second cardiovascular therapeutic.

2. An enucleated erythroid cell, comprising a first exogenous polypeptide comprising a first metabolic therapeutic, and a second exogenous polypeptide, comprising a second metabolic therapeutic.

3. The enucleated erythroid cell of claim 1 or 2, wherein the first and second exogenous polypeptides have agent-additive, agent-synergistic, multiplicative, independent function, localization-based, proximity-dependent, scaffold-based, multimer-based, pathway-based, or compensatory activity.

4. The enucleated erythroid cell of claim 1, which further comprises a third exogenous polypeptide that comprises a third cardiovascular therapeutic.

5. The enucleated erythroid cell of claim 2, which further comprises a third exogenous polypeptide that comprises a third metabolic therapeutic.

6. The enucleated erythroid cell of any of the preceding claims, wherein:
a) the first and second exogenous polypeptides act on the same target, e.g., a clotting factor, wherein optionally the target is a cell surface receptor and/or an endogenous human protein;
b) the first exogenous polypeptide binds to a first endogenous human protein and the second exogenous polypeptide binds to a second endogenous human target protein, e.g., with a Kd of less than 500, 200, 100, 50, 20, 10, 5, 2, or 1 nM;
c) the first exogenous polypeptide (e.g., an enzyme) acts on (e.g., binds) a first target, and the second exogenous polypeptide (e.g., an enzyme) act on (e.g., binds) a second target, wherein the first and second targets are members of the same biological pathway, wherein optionally the targets are cell surface receptors, endogenous human proteins (e.g., enzymes), or both;
d) the first and second exogenous polypeptides are in close proximity to each other, e.g., are less than 10, 7, 5, 4, 3, 2, 1, 0.5, 0.2, or 0.1 nm apart for a duration of at least 1, 2, 5, 10, 30, or 60 seconds; 1, 2, 5, 10, 30, or 60 minutes, or 1, 2, 3, 6, 12, or 14 hours;
e) the first and second exogenous polypeptides have a Kd of less than 500, 200, 100, 50, 20, 10, 5, 2, or 1 nM for each other;
f) the first and second exogenous polypeptides act on different targets (e.g., clotting factors), wherein optionally at least one of the targets is a cell surface receptor and/or an endogenous human protein, e.g., the first exogenous polypeptide binds a first cell type e.g., an immune effector cell, and the second exogenous polypeptide binds a second cell type, e.g., an immune effector cell, e.g., a T cell;
g) the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number;
h) the first exogenous polypeptide and the second exogenous polypeptide have a Kd for a first target and a second target, respectively, with a ratio of about 1:1, from about 2:1 to 1:2, from about 5:1 to 1:5, from about 10:1 to 1:10, from about 20:1 to 1:20, from about 50:1 to 1:50, from about 100:1 to 1:100;
i) the first exogenous polypeptide has a first activity (e.g., binding) towards a first target, and the second exogenous polypeptide has a second activity (e.g., binding) towards the first target, e.g., the first and second exogenous polypeptides bind a single target;
j) the first exogenous polypeptide acts on (e.g., binds) a first target and the second exogenous polypeptide acts on (e.g., binds) a second target, and the first and second targets are part of the same pathway, wherein optionally the first exogenous polypeptide acts on the first target and the second exogenous polypeptide acts on the second target simultaneously;
k) the first exogenous polypeptide acts on (e.g., binds) a first target and the second exogenous polypeptide acts on (e.g., binds) a second target, and the first and second targets are part of different pathways, wherein optionally the first and second pathways both act to promote a given cellular response;
l) the first exogenous polypeptide localizes the enucleated erythroid cell to a desired site, e.g., a blood clot, and the second exogenous polypeptide has a therapeutic activity, e.g., a fibrinolytic enzyme;
m) the first exogenous polypeptide binds a first cell, e.g., a first cell type, and the second exogenous polypeptide binds a second cell, e.g., a second cell type, e.g., an immune effector cell, e.g., a T cell;
n) the first exogenous polypeptide and the second exogenous polypeptide are non-human proteins, e.g., the enzymes are not natively found in humans;
o) the first exogenous polypeptide and the second exogenous polypeptide are both enzymes, e.g., biosynthetic enzymes;
p) the first exogenous polypeptide (e.g., an enzyme) promotes formation of an intermediate molecule (e.g., converts a substrate into an intermediate) and the second exogenous polypeptide (e.g., an enzyme) acts on the intermediate molecule (e.g., converts an intermediate into a product);
q) the first exogenous polypeptide and the second exogenous polypeptide act on successive steps of a pathway;
r) the erythroid cell comprises at least at least 10 copies, 100 copies, 1,000 copies, 5,000 copies 10,000 copies, 25,000 copies, 50,000 copies, or 100,000 copies of each of the first exogenous polypeptide and the second exogenous polypeptide;
s) the copy number of the first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the second exogenous polypeptide; or
t) the copy number of the second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the first exogenous polypeptide.

7. The enucleated erythroid cell of any of the preceding claims, wherein the first exogenous polypeptide comprises an anti-PCSK9 antibody molecule or a kallikrein inhibitor (e.g., ecallantaide) or a fragment or variant thereof.

8. The enucleated erythroid cell of any of the preceding claims, wherein the first exogenous polypeptide and the second exogenous polypeptide are enzymes (e.g., enzymes that are not natively found in humans).

9. The enucleated erythroid cell of any of the preceding claims, wherein the first exogenous polypeptide comprises phenylalanine ammonia lyase (PAL) or a phenylalanine-metabolizing fragment or variant thereof, wherein optionally the second exogenous polypeptide comprises an enzyme.

10. The enucleated erythroid cell of any of the preceding claims, wherein the first exogenous polypeptide acts on (e.g., binds) a clotting factor (e.g., the first exogenous polypeptide is an antibody for the target) and the second exogenous polypeptide activates or inactivates (e.g., cleaves) the clotting factor (e.g., Tissue Factor, Factor V, Factor VII, Factor VIII, Factor IX, Factor X, Factor XI, Factor XIII, thrombin, or fibrinogen).

11. The enucleated erythroid cell of any of the preceding claims, wherein the second exogenous polypeptide comprises a clotting factor that acts on a target (e.g., a substrate), wherein optionally the second exogenous polypeptide comprises Tissue Factor and the target comprises Factor VII; the second exogenous polypeptide comprises TF-VIIa and/or Factor IXa and the target comprises Factor X; the second exogenous polypeptide comprises Factor XIa and the target comprises Factor IX; the second exogenous polypeptide comprises TF-VIIa and the target comprises Factor IX; the second exogenous polypeptide comprises Factor VIIIa and the target comprises Factor X; the second exogenous polypeptide comprises Factor XI; Factor VIII, or Factor V and the target comprises thrombin; the second exogenous polypeptide comprises Factor Va or Xa and the target comprises prothrombin; the second exogenous polypeptide comprises thrombin and the target comprises fibrinogen or Factor XIII; or the second exogenous polypeptide comprises a plasminogen activator (e.g., urokinase or tissue plasminogen activator (TPA)) and the target comprises plasminogen.

12. The enucleated erythroid cell of any of the preceding claims, wherein the first exogenous polypeptide acts on (e.g., binds) a blood clot (e.g., the first exogenous polypeptide comprises an anti-fibrin antibody molecule, fibrin, or a fibrin-binding portion or variant thereof) and the second exogenous polypeptide is a fibrinolytic enzyme (e.g., plasmin or a fibrinolytic fragment or variant thereof).

13. The erythroid cells of any of claim 1, 3, 4, or 6 for use in a method of treating a subject having a cardiovascular disease, wherein the erythroid cells are administered to the subject in an amount effective to treat the cardiovascular disease.

14. The erythroid cells for use of claim 13, wherein the cardiovascular disease is hypercholesterolemia or hereditary angioedema.

15. The erythroid cells of any of claim 2, 3, 5, or 6 for use in a method of treating a subject having a metabolic disorder (e.g., a metabolic deficiency), wherein the erythroid cells are administered to the subject in an amount effective to treat the metabolic disorder (e.g., a metabolic deficiency).

16. The erythroid cells for use of claim 15, wherein the metabolic disorder (e.g., a metabolic deficiency) is selected from the group consisting of hemophilia (e.g., hemophilia type A, hemophilia type B, or hemophilia type C), von Willebrand disease, Factor II deficiency, Factor V deficiency, Factor VII deficiency, Factor X deficiency, Factor XII deficiency, thrombotic thrombocytopenic purpura, Phenylketonuria (PKU), Adenosine Deaminase Deficiency-Severe Combined Immunodeficiency (ADA-SCID), Mitochondrial Neurogastrointestinal Encephalopathy (MNGIE), Primary Hyperoxaluria, Alkaptonuria, and Thrombotic Thrombocytopenic Purpura (TTP).

17. The erythroid cells for use of claim 15 or 16, wherein the first exogenous polypeptide acts on (e.g., binds) a clotting factor and the second exogenous polypeptide activates or inactivates (e.g., cleaves) the clotting factor (e.g., Tissue Factor, Factor V, Factor VII, Factor VIII, Factor IX, Factor X, Factor XI, Factor XIII, thrombin, or fibrinogen), wherein optionally the second exogenous polypeptide comprises an activated clotting factor (e.g., Tissue Factor, Factor V, Factor VII, Factor VIII, Factor IX, Factor X, Factor XI, Factor XIII, thrombin, or fibrinogen).

18. The erythroid cells for use of claim 17, wherein the subject has a clotting deficiency disease such as hemophilia (e.g., hemophilia type A, hemophilia type B, or hemophilia type C), von Willebrand disease, Factor II deficiency, Factor V deficiency, Factor VII deficiency, Factor X deficiency, or Factor XII deficiency.

19. The erythroid cells for use of claim 15 or 16, wherein the second exogenous polypeptide comprises a clotting factor that acts on a target, wherein optionally the second exogenous polypeptide comprises Tissue Factor and the target comprises Factor VII; the second exogenous polypeptide comprises TF-VIIa or Factor IXa and the target comprises Factor X; the second exogenous polypeptide comprises Factor XIa and the target comprises Factor IX; the second exogenous polypeptide comprises TF-VIIa and the target comprises Factor IX; the second exogenous polypeptide comprises Factor VIIIa and the target comprises Factor X; the second exogenous polypeptide comprises Factor XI; Factor VIII, or Factor V and the target comprises thrombin (e.g., Factor IIa); the second exogenous polypeptide comprises Factor Va or Xa and the target comprises prothrombin; the second exogenous polypeptide comprises thrombin (e.g., Factor IIa) and the target comprises fibrinogen or Factor XIII; or the second exogenous polypeptide comprises a plasminogen activator (e.g., urokinase or tissue plasminogen activator (TPA)) and the target comprises plasminogen.

20. The erythroid cells for use of claim 19, wherein the second exogenous polypeptide reduces unwanted clotting in a subject having or at risk of developing a blood clot such as the subject has, or is at risk of developing, thrombophilia, pulmonary embolism, or stroke.

21. The erythroid cells for use of claim 19, wherein the metabolic disorder (e.g., a metabolic deficiency) is hemophilia A, the second exogenous polypeptide comprises Factor VIII or fragment thereof, and the target is thrombin (e.g., Factor IIa) or Factor X.

22. The erythroid cells for use of claim 19, wherein the metabolic disorder (e.g., a metabolic deficiency) is hemophilia B, the first exogenous polypeptide binds Factor XIa or factor X or a fragment thereof (e.g., is an antibody for Factor XIa or Factor X or a fragment thereof), and the second exogenous polypeptide comprises factor IX or fragment thereof.

23. The erythroid cells for use of claim 15 or 16, wherein the metabolic disorder (e.g., a metabolic deficiency) is thrombotic thrombocytopenic purpura, the first exogenous polypeptide binds ultra-large von Willebrand factor (ULVWF) or fragment thereof (e.g., the first exogenous polypeptide is an antibody for ULVWF), and the second exogenous polypeptide comprises ADAMTS13 or fragment thereof.

24. The erythroid cells for use of claim 15 or 16, wherein the first exogenous polypeptide acts on (e.g., binds) a blood clot and the second exogenous polypeptide comprises a fibrinolytic enzyme (e.g., plasmin or a fibrinolytic fragment or variant thereof), wherein optionally the first exogenous polypeptide is selected from the group consisting of an anti-fibrin antibody molecule, fibrin, and a fibrin-binding portion or variant thereof.

25. The erythroid cells for use of claim 15 or 16, wherein the metabolic disorder (e.g., a metabolic deficiency) is PKU, the first exogenous polypeptide comprises phenylalanine ammonia lyase (PAL) or a phenylalanine-metabolizing fragment or variant thereof, and the second exogenous polypeptide comprises an enzyme.

26. The erythroid cells for use of claim 15 or 16, wherein the metabolic disorder (e.g., a metabolic deficiency) is ADA-SCID and the first exogenous polypeptide comprises adenosine deaminase (ADA) or a fragment or variant thereof.

27. The erythroid cells for use of claim 15 or 16, wherein the metabolic disorder (e.g., a metabolic deficiency) is Mitochondrial Neurogastrointestinal Encephalopathy and the first exogenous polypeptide comprises thymidine phosphorylase or a fragment or variant thereof.

28. The erythroid cells for use of claim 15 or 16, wherein the metabolic disorder (e.g., a metabolic deficiency) is Primary Hyperoxaluria and the first exogenous polypeptide comprises oxalate oxidase or a fragment or variant thereof.

29. The erythroid cells for use of claim 15 or 16, wherein the metabolic disorder (e.g., a metabolic deficiency) is Alkaptonuria and the first exogenous polypeptide comprises homogentisate oxidase or a fragment or variant thereof.

30. The erythroid cells for use of claim 15 or 16, wherein the metabolic disorder (e.g., a metabolic deficiency) is Thrombotic Thrombocytopenic Purpura and the first exogenous polypeptide comprises ADAMTS13 or a fragment or variant thereof.

31. The erythroid cell or erythroid cells for use of any of the preceding claims, wherein the first exogenous polypeptide binds to a target more strongly than the first exogenous polypeptide binds to the second exogenous polypeptide.

32. The erythroid cell or erythroid cells for use of any of the preceding claims, wherein the first exogenous polypeptide promotes fusion of the erythroid cell with a target cell.

33. The erythroid cell or erythroid cells for use of any of the preceding claims, wherein the cell comprises at least 2 but no more than 5, 6, 7, 8, 9, or 10 different exogenous polypeptides, e.g., exogenous polypeptides that are encoded by one or more exogenous nucleic acids that are not retained by the enucleated erythroid cell.

34. The erythroid cell or erythroid cells for use of any of the preceding claims, wherein the exogenous polypeptides are encoded by one or more exogenous nucleic acids that are not retained by the enucleated erythroid cell.

35. The erythroid cell or erythroid cells for use of any of the preceding claims, wherein one or more (e.g., two or three) of the first, second, and optionally third exogenous polypeptides are transmembrane polypeptides or surface-anchored polypeptides.

36. The erythroid cell or erythroid cells for use of any of the preceding claims, wherein both the first and second exogenous polypeptides have a stoichiometric mode of action, or both have a catalytic mode of action, and both are present at a similar abundance, e.g., about 1:1 or from about 2:1 to 1:2.

37. The erythroid cell or erythroid cells for use of any of the preceding claims, wherein the first exogenous polypeptide is more abundant than the second exogenous polypeptide by at least about 10%, 20%, 30%, 50%, or a factor of 2, 3, 4, 5, 10, 20, 50, or 100 (and optionally up to 10 or 100 fold) by weight or copy number.

38. The erythroid cell or erythroid cells for use of any of the preceding claims, wherein the first polypeptide has a stoichiometric mode of action and the second polypeptide has a catalytic mode of action, and the first polypeptide is more abundant than the second polypeptide.

39. The erythroid cell or erythroid cells for use of any of the preceding claims, wherein the cell has one or more of the following characteristics:
a) an osmotic fragility of less than 50% cell lysis at 0.3%, 0.35%, 0.4%, 0.45%, or 0.5% NaCl;
b a cell volume of about 10-200 fL or a cell diameter of between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns, or between about 10 microns and about 30 microns;
c) greater than 1%, 2%, 3%, 4%, 5%, 6%, 7%, 8%, 9% or 10% fetal hemoglobin; or at least about 20, 25, or 30 pg/cell of hemoglobin; or d) phosphatidylserine content of the outer leaflet is less than 30%, 25%, 20%, 15%, 10%, or 5% as measured by Annexin V staining.

40. The erythroid cell or erythroid cells for use of any of the preceding claims, wherein at least one, e.g., all, of the plurality of exogenous polypeptides are glycosylated.

41. The erythroid cell or erythroid cells for use of any of the preceding claims, wherein the exogenous polypeptide or polypeptides lack a sortase transfer signature such as LPXTG.

42. The erythroid cell or erythroid cells for use of any of the preceding claims, wherein:
i) at least 50, 60, 70, 80, 90, 95, or 99% of the exogenous polypeptides, e.g., fusion proteins on the surface of the erythroid cell have an identical sequence,
ii) at least 50, 60, 70, 80, 90, 95, or 99% of the exogenous polypeptides, e.g., fusion proteins have the same transmembrane region,
iii) the first and/or second exogenous polypeptide, e.g., fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;
iv) at least 50, 60, 70, 80, 90, 95, or 99% of the exogenous polypeptides, e.g., fusion proteins do not differ from one another by more than 1, 2, 3, 4, 5, 10, 20, or 50 amino acids,
v) the first and/or second exogenous polypeptide lacks a sortase transfer signature,
vi) the first and/or second exogenous polypeptide comprises a moiety that is present on less than 1, 2, 3, 4, or 5 sequence distinct fusion polypeptides;
vii) the first and/or second exogenous polypeptide is present as a single fusion polypeptide;
viii) the first and/or second exogenous polypeptide, e.g., fusion protein does not contain Gly-Gly at the junction of an endogenous transmembrane protein and the moiety;
ix) the first and/or second exogenous polypeptide, e.g., fusion protein does not contain Gly-Gly, or the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment; or a combination thereof.

43. The erythroid cell or erythroid cell for use according to any of the preceding claims, wherein the first exogenous polypeptide interacts with a target, and the second exogenous polypeptide (e.g., IdeS) modifies the target.

44. A method of making an erythroid cell according to any of the proceeding claims, comprising:
a) providing an erythroid cell, and
b) contacting the erythroid cell with nucleic acid encoding the first exogenous protein and nucleic acid encoding the second exogenous protein, under conditions that allow uptake of the nucleic acid by the erythroid cell, and
c) culturing the cell under conditions that allow for expression of the first and second exogenous proteins,
thereby making an erythroid cell of any of the proceeding claims.

45. The method of claim 44, wherein the nucleic acid encoding the first exogenous protein and the nucleic acid encoding the second exogenous protein are separate nucleic acids.

46. The method of claim 44, wherein the nucleic acid encoding the first exogenous protein and the nucleic acid encoding the second exogenous protein are part of the same nucleic acid molecule.

47. A plurality of erythroid cells according to any of the proceeding claims, e.g., wherein the plurality comprises at least 10⁸, 10⁹, 10¹⁰, 10¹¹, or 10¹² erythroid cells according to any of the proceeding claims.

48. A pharmaceutical composition comprising a cell according to any of claims 1-4, or a plurality of cells according to claim 47.

49. An enucleated erythroid cell, comprising a first exogenous polypeptide comprising a first cardiovascular therapeutic, and a second exogenous polypeptide, comprising a second cardiovascular therapeutic, wherein:
the first cardiovascular therapeutic comprises a naturetic peptide (e.g., BNP) and the second cardiovascular therapeutic comprises relaxin (e.g., relaxin 2);
the first cardiovascular therapeutic comprises a biomarker of cardiac inflammation (e.g., myeloperoxidase (MPO)) and the second cardiovascular therapeutic comprises relaxin (e.g., relaxin 2); or
the first cardiovascular therapeutic comprises a plasminogen activator (e.g., tissue plasminogen activator (TPA)) and the second cardiovascular therapeutic comprises a tissue factor pathway inhibitor (TFPI).

50. The erythroid cells of claim 49 for use in a method of treating a subject having a cardiovascular disease (e.g., heart failure, atherosclerosis, or thromboembolism), wherein the erythroid cells are administered to the subject in an amount effective to treat the cardiovascular disease (e.g., heart failure, atherosclerosis, or thromboembolism).

51. An enucleated erythroid cell, comprising a first exogenous polypeptide comprising a first metabolic therapeutic, and a second exogenous polypeptide, comprising a second metabolic therapeutic, wherein:
the first metabolic therapeutic comprises an agent that binds a pancreatic beta cell receptor (e.g., GLP-1) and the second metabolic therapeutic comprises an agent that regulates glucose metabolism (e.g., insulin);
the first metabolic therapeutic comprises a fibroblast growth factor (e.g., fibroblast growth factor 21 (FGF-21)) and the second metabolic therapeutic comprises an agent that binds a glucagon receptor (e.g., a secretin, e.g., glucagon);
the first metabolic therapeutic comprises leptin and the second metabolic therapeutic comprises an agent that binds a glucagon receptor (e.g., a secretin, e.g., glucagon);
the first metabolic therapeutic comprises an agent that targets an osteoclast receptor e.g., RANK-L (e.g., a RANK-L antibody, e.g., denusomab) and the second metabolic therapeutic comprises an osteoclast activator, e.g., a ligand for parathyroid hormone 1 receptor, e.g., parathyroid hormone (PTH);
the first metabolic therapeutic comprises a cystathionine B-synthase (CBS) polypeptide and the second metabolic therapeutic comprises an L-homocysteine or L-serine transporter, e.g., sodium-coupled neutral amino acid transporter 2 (SAT2) or neutral amino acid transporter A (ASCT1); or
the first metabolic therapeutic comprises an uricase polypeptide and the second metabolic therapeutic comprises a catalase polypeptide.

52. The erythroid cells of claim 51 for use in a method of treating a subject having a metabolic disorder (e.g., a metabolic deficiency (e.g., homocystinuria or hyperuricemia) or a metabolic disorder selected from diabetes (e.g., type 1 diabetes, type 2 diabetes, or gestational diabetes), insulin insensitivity, and osteoporosis), wherein the erythroid cells are administered to the subject in an amount effective to treat the metabolic disorder (e.g., a metabolic deficiency (e.g., homocystinuria or hyperuricemia) or a metabolic disorder selected from diabetes (e.g., type 1 diabetes, type 2 diabetes, or gestational diabetes), insulin insensitivity, and osteoporosis).
